(19) United States
(12) Patent Application Publication
(10) Pub. No.: US 2018/0265899 A1
(43) Pub. Date: Sep. 20, 2018

(54) CARBON DIOXIDE FIXATION DEVICE AND FUEL PRODUCTION SYSTEM

(71) Applicant: KABUSHIKI KAISHA TOSHIBA, Minato-ku (JP)

(72) Inventors: Yasuko NORITOMI, Kawasaki (JP); Takashi KUBOKI, Tokyo (JP)

(73) Assignee: KABUSHIKI KAISHA TOSHIBA, Minato-ku (JP)

(21) Appl. No.: 15/914,422

(22) Filed: Mar. 7, 2018

(30) Foreign Application Priority Data
Mar. 16, 2017 (JP) 2017-051443
Jan. 12, 2018 (JP) 2018-003345

(51) Int. Cl. C12P 7/04; C10L 1/02

(52) U.S. Cl. CPC C12P 7/04; C10L 2290/38; C10L 2290/26; C10L 1/02

(57) ABSTRACT

According to one embodiment, provided is a carbon dioxide fixation device including a nonaqueous phase and an aqueous phase. The nonaqueous phase includes an ionic liquid, an enzyme body, and a mediator. The enzyme body catalyzes a reduction reaction of carbon dioxide or a reduced product of carbon dioxide. The mediator acts as a reducing agent or coenzyme in this reduction reaction. The nonaqueous phase is configured such that the reduction reaction generates a reaction product. The aqueous phase includes an extraction liquid containing water. The aqueous phase is configured such that the abovementioned reaction product is supplied thereto from the nonaqueous phase.

### CROSS-REFERENCE TO RELATED APPLICATIONS

This application is based upon and claims the benefit of priority from Japanese Patent Application No. 2017-051443 filed Mar. 16, 2017 and Japanese Patent Application No. 2018-003345 filed Jan. 12, 2018; the entire contents of all of which are incorporated herein by reference.

### FIELD

Embodiments described herein relate generally to a carbon dioxide fixation device and fuel production system.

### BACKGROUND

Coal consumption which keeps increasing yearly has raised the carbon dioxide (CO₂) concentration in the atmosphere, and has become a main cause of global warming. In the natural world, photosynthesis converts about one hundred billion tons of CO₂ into biomass in a year. However, the CO₂ emission amount has kept increasing yearly since the Industrial Revolution. As a consequence, the CO₂-biomass conversion rate cannot catch up with the CO₂ emission rate any longer by natural photosynthesis alone.

As one global warming measure, therefore, it has become necessary to capture and store CO₂ being a major cause of greenhouse gas.

Various measures have been made even up to now. Examples are chemical absorption/separation of CO₂ by an inorganic chemical (e.g., an alkaline solution such as amines), physical absorption/separation of CO₂ by an absorption solution such as methanol or polyethylene glycol, separation of CO₂ by membrane separation using a polymeric membrane or ceramics membrane, physical absorption/separation of CO₂ by a porous absorption material such as zeolite or activated carbon, and a method of compressing CO₂ and storing the compressed CO₂ underground or at the bottom of the sea. Of CCS (Carbon Dioxide Capture and Storage) techniques, for example, a technique of separating and recovering CO₂ generated from a thermal power plant, transporting the recovered CO₂, and storing the transported CO₂ underground or at the bottom of the sea is being promoted. Unfortunately, this technique poses problems such as a high cost of CO₂ separation and recovery.

### BRIEF DESCRIPTION OF THE DRAWINGS

FIG. 1 is a schematic view of a carbon dioxide fixation device of an example according to a mode of an embodiment;

FIG. 2 is a schematic view of a carbon dioxide fixation device of another example according to the mode of the embodiment;

FIG. 3 is a schematic view of a carbon dioxide fixation device of an example according to another mode of the embodiment;

FIG. 4 is a schematic view of a carbon dioxide fixation device of another example according to the other mode of the embodiment;

FIG. 5 is a schematic view of an exemplary modification of the carbon dioxide fixation device shown in FIG. 4;

FIG. 6 is a schematic view of another exemplary modification of the carbon dioxide fixation device shown in FIG. 4;

FIG. 7 is a schematic view of a carbon dioxide fixation device of still another example according to the other mode of the embodiment;

FIG. 8 is a schematic view of a carbon dioxide fixation device of yet another example according to the other mode of the embodiment;

FIG. 9A is a schematic view showing an intermediate state during manufacture of an example of a member included in the carbon dioxide fixation device shown in FIG. 8;

FIG. 9B is a schematic view showing an intermediate state during manufacture of another example of the member included in the carbon dioxide fixation device shown in FIG. 8;

FIG. 9C is a schematic view showing an intermediate state during manufacture of still another example of the member included in the carbon dioxide fixation device shown in FIG. 8;

FIG. 10 is a schematic view of a carbon dioxide fixation device of still another example according to the mode of the embodiment;

FIG. 11A is a schematic plan sectional view of a single-cell for forming a stacked carbon dioxide fixation device of an example according to the embodiment;

FIG. 11B is a schematic side sectional view of the single-cell for forming the stacked carbon dioxide fixation device of the example according to the embodiment;

FIG. 12 is a schematic view of a stacked carbon dioxide fixation device of an example according to the embodiment;

FIG. 13 is a scheme showing a fuel production system of an example according to an embodiment; and

FIG. 14 is a schematic view of an exemplary modification of a carbon dioxide fixation device of an example according to the mode of the embodiment.

### DETAILED DESCRIPTION

According to one embodiment, provided is a carbon dioxide fixation device including a nonaqueous phase and an aqueous phase. The nonaqueous phase includes an ionic liquid, an enzyme body, and a mediator. The enzyme body catalyzes a reduction reaction of carbon dioxide or a reduced product of carbon dioxide. The mediator acts as a reducing agent or coenzyme in this reduction reaction. The nonaqueous phase is configured such that the reduction reaction generates a reaction product. The aqueous phase includes an extraction liquid containing water. The aqueous phase is configured such that the abovementioned reaction product is supplied thereto from the nonaqueous phase.

According to another embodiment, provided is a fuel production system including a fuel generation section, a carbon dioxide supply section, and an extraction liquid recovery section. The fuel generation section includes the carbon dioxide fixation device according to the above embodiment. The carbon dioxide supply section is configured to supply carbon dioxide to the fuel generation section. The extraction liquid recovery section is configured to recover a reaction product from the fuel generation section.

Recently, as next stages aiming at the realization of a sustainable society, it has become necessary, in addition to the prevention of global warming, not only to capture and store CO₂, but also to develop and establish bioprocesses such as biorefinery which actively converts CO₂ into a useful substance such as chemical fuel or biomass, and fixation of CO₂.

Also, techniques of performing gelation and membrane formation on an ionic liquid capable of efficiently absorbing and separating CO₂ are recently beginning to be established. On the other hand, extensive research is undergoing for actively converting captured CO₂ into useful fuel using electrochemical conversion or photochemical conversion, as conversion method. As electrochemical conversion, for example, it has been reported that ethanol can be produced from carbon dioxide with high selectivity and high Faraday efficiency at standard temperature and pressure by using, as both an electrode and catalyst, a material obtained by modifying spike-form graphene with spherical copper nanoparticles, without using any noble metal or rare metal. This method can manufacture chemical fuel using electrical energy obtained by wind power generation, solar power generation, or the like, and hence is a very attractive chemical fuel manufacturing method. This manufacturing method attracts great attention also from the needs for energy conversion and storage. However, CO₂ is only slightly soluble in water, and this makes it difficult to produce high-concentration ethanol.

The enzyme conversion method is recently attracting attention again as a clean, high-efficiency fuel production method capable of selectively converting CO₂ into fuel under mild conditions. However, conventional enzyme conversion method have problems such as the loss of an intermediate product during the production of fuel (methanol) performed by a cascade enzymatic reaction. Also, there are various problems such as a low purity and low concentration of manufactured methanol, and denaturation and outflow of enzyme(s). Like the electrochemical method, one fundamental cause of these problems is presumably a low solubility of CO₂, which is the raw material for fuel production, into an aqueous solution as reaction solvent.

### First Embodiment

The first embodiment provides a carbon dioxide fixation device including a nonaqueous phase and an aqueous phase. The nonaqueous phase contains one or more kinds of ionic liquids, one or more kinds of enzyme bodies, and one or more kinds of mediators. The aqueous phase contains an extraction liquid.

The nonaqueous phase and aqueous phase are in contact with each other with, e.g., one or more kinds of porous membranes or separators being interposed therebetween. The nonaqueous phase and aqueous phase may alternatively be in direct contact with each other.

Carbon dioxide (CO₂) is supplied to the nonaqueous phase in the carbon dioxide fixation device according to the embodiment. When using an ionic liquid as a medium of the nonaqueous phase, the nonaqueous phase can selectively and efficiently absorb CO₂.

The supply source of carbon dioxide is, e.g., a gas containing carbon dioxide. The carbon dioxide supply source is not particularly limited as long as the source is a gas containing carbon dioxide. For example, it is possible to use, as the carbon dioxide supply source, atmospheric gas, a gas generated in a thermal power plant, an exhaust gas, a gas from dry ice, or a gas from a carbon dioxide gas cylinder.

The carbon dioxide fixation device according to the embodiment may further include a mechanism configured to bring the abovementioned gas containing carbon dioxide into contact with, e.g., the nonaqueous phase. Alternatively, the device may be configured such that the nonaqueous phase is exposed to the atmosphere. In such manner, it is possible to appropriately design the carbon dioxide fixation device in accordance with the source of the gas containing carbon dioxide as a reactant.

The carbon dioxide fixation device may also include a mechanism which supplies the gas containing carbon dioxide to the nonaqueous phase in a state in which the gas is pressurized, e.g., to a pressure higher than atmospheric pressure (0.101325 MPa=1 atm). The rate of supply of carbon dioxide to the nonaqueous phase may increase when the pressure of the gas, i.e., the pressure of carbon dioxide is raised.

Each of the one or more kinds of enzyme bodies contained in the nonaqueous phase contains at least one enzyme. As will be described in detail later, carbon dioxide supplied to the nonaqueous phase is reduced by enzymatic reaction(s), and this reduction generates fuel, for example. Accordingly, a section in the carbon dioxide fixation device including the nonaqueous phase may also be referred to as a carbon dioxide reduction reaction section.

Each of the one or more kinds of mediators contained in the nonaqueous phase is a mediator for the reduction reaction(s) which is catalyzed by the enzyme body(s). More specifically, the mediator acts as a reducing agent or coenzyme in the reduction reaction(s). In the reduction reaction(s), the mediator reduces carbon dioxide or a reduced product thereof, and the mediator itself is oxidized.

When the mediator participates in the reduction reaction as a reducing agent or coenzyme, electrons may transfer from the mediator to the reaction site of the reduction reaction. The electrons having transferred to the reaction site may be used in the reduction reaction. Also, the mediator may become oxidized as the mediator gives up electrons.

Furthermore, the enzymatic reaction which reduces carbon dioxide may be a cascade reaction. In this case, the nonaqueous phase contains an enzyme body which catalyzes an enzymatic reaction for further reducing the reduced product of carbon dioxide. Carbon dioxide may be reduced to a final product through plural cascades of the reduction reaction.

In addition, the enzyme bodies contained in the nonaqueous phase may include an enzyme body which catalyzes the reduction reaction of the oxidized mediator (e.g., an oxidant of the mediator). The enzyme body which catalyzes the reduction reaction of the oxidized mediator and the enzyme body which catalyzes the reduction reaction of carbon dioxide may be different enzyme bodies. Alternatively, the enzyme body which reduces the oxidized mediator and the enzyme body which reduces carbon dioxide may be the same enzyme body. For example, one enzyme body may contain both an enzyme which catalyzes the reduction reaction of the mediator and an enzyme which catalyzes the reduction reaction of carbon dioxide. Likewise, the enzyme body which catalyzes the reduction reaction of the oxidized mediator and the enzyme body which catalyzes the reduction reaction of a reduced product of carbon dioxide may be different enzyme bodies or the same enzyme body. For example, one enzyme body may contain both an enzyme which catalyzes the reduction reaction of the mediator and an enzyme which catalyzes the reduction reaction of the reduced product of carbon dioxide.

The reaction product generated in the nonaqueous phase is supplied to the aqueous phase. The extraction liquid contained in the aqueous phase selectively extracts the reaction product generated in the nonaqueous phase. In one example, the extraction liquid of the aqueous phase selectively extracts and recovers the final product in the cascade enzymatic reaction of carbon dioxide. Accordingly, in the carbon dioxide fixation device, the aqueous phase may be referred to as a fuel recovery section.

Note that as described above, the carbon dioxide fixation device according to the embodiment includes the carbon dioxide reduction reaction section configured to generate fuel by reducing carbon dioxide, and the fuel recovery section configured to recover the generated fuel. Therefore, the carbon dioxide fixation device according to the embodiment may also be referred to as a fuel production device.

Fixation of carbon dioxide herein mentioned means conversion from carbon dioxide to a carbon compound by a chemical reaction. Carbon dioxide as a culprit gas of global warming can be reduced by converting carbon dioxide into a gaseous, liquid, or solid carbon compound by chemical reaction. Also, carbon dioxide can be used as a resource by obtaining a useful substance such as fuel as the carbon compound.

Note that, carbon dioxide reduction is included in the above-described conversion of carbon dioxide into a carbon compound by a chemical reaction.

In the carbon dioxide fixation device according to the embodiment, the section which generates the reaction product by reducing the reactant (carbon dioxide or its reduced product) and the section which recovers the reaction product (the carbon compound as the reduced product of carbon dioxide) are divided into the nonaqueous phase and aqueous phase. Hence, the reactant hardly mixes in the reaction product, and the reaction product can thus be recovered with a small impurity amount.

Also, in the carbon dioxide fixation device according to the embodiment, the aqueous phase extracts the reaction product (e.g., fuel) from the nonaqueous phase including the reaction field. Therefore, the equilibrium of the enzymatic reaction of fuel generation can be shifted to the reaction product side. In addition, the loss of the reaction product reduces because the aqueous phase extracts the reaction product from the nonaqueous phase. Consequently, the yield of the reaction product (e.g., fuel) generated can be raised.

As described above, the carbon dioxide fixation device according to the embodiment achieves a high performance of carbon dioxide fixation by efficiently performing the reaction of reducing carbon dioxide or its reduced product to a reaction product such as methanol. This makes it possible to suppress CO₂ emission, and at the same time produce compounds useful as fuel or the like.

In the first mode of the carbon dioxide fixation device according to the embodiment, the nonaqueous phase may include first, second, and third nonaqueous phases, and the aqueous phase may include first, second, and third aqueous phases. That is, the carbon dioxide fixation device of this mode may include a plurality of nonaqueous phases, and a plurality of aqueous phases.

Carbon dioxide is supplied to the first nonaqueous phase. In the first nonaqueous phase, a reduction reaction of the supplied carbon dioxide generates a first reaction product. In one example, the first reaction product is formic acid.

Formic acid as described herein means any form among formate anion (HCOO⁻), formic acid (HCOOH) where a proton has bonded to formate anion, or a formate salt where a cation has bonded to formate anion. Formic acid as described in the following description includes any of the above-described form, as well. For other compounds also, the description is meant to include any form such as among anion, acid, or salt.

The first reaction product is supplied from the first nonaqueous phase to the first aqueous phase. Also, the first aqueous phase supplies the first reaction product to the second nonaqueous phase.

The first reaction product is supplied to the second nonaqueous phase. In the second nonaqueous phase, a reduction reaction of the supplied first reaction product generates a second reaction product. In one example, the second reaction product is formaldehyde.

The second nonaqueous phase supplies the second reaction product to the second aqueous phase. Also, the second aqueous phase supplies the second reaction product to the third nonaqueous phase.

The second reaction product is supplied from the second aqueous phase to the third nonaqueous phase. In the third nonaqueous phase, a reduction reaction of the supplied second reaction product generates a third reaction product. In one example, the third reaction product is methanol. The third reaction product generated in the third nonaqueous phase is supplied to the third aqueous phase.

The third reaction product is supplied from the third nonaqueous phase to the third aqueous phase. The third reaction product is thus obtained.

In this mode, the three-cascade enzymatic reaction in the first to third nonaqueous phases reduces carbon dioxide to a final product (fuel). That is, the first and second reaction products are intermediate products of the three-cascade reaction, and the third reaction product is the final product.

Each of the first to third nonaqueous phases may contain one or more kinds of ionic liquids, one or more kinds of enzyme bodies, and one or more kinds of mediators. The kinds and numbers of ionic liquids, enzyme bodies, and mediators contained in the first to third nonaqueous phases may be different from each other, or may alternatively be the same.

The enzyme body(s) contained in the first nonaqueous phase includes enzyme(s) which catalyzes the reduction reaction of generating the first reaction product by reducing carbon dioxide. Also, the mediator(s) contained in the first nonaqueous phase acts as reducing agent(s) or coenzyme(s) in the reduction reaction of generating the first reaction product.

The enzyme body(s) contained in the second nonaqueous phase may include enzyme(s) which catalyzes the reduction reaction of generating the second reaction product by reducing the first reaction product. Also, the mediator(s) contained in the second nonaqueous phase acts as reducing agent(s) or coenzyme(s) in the reduction reaction of generating the second reaction product.

Likewise, the enzyme body(s) contained in the third nonaqueous phase includes enzyme(s) which catalyzes the reduction reaction of generating the third reaction product by reducing the second reaction product. Also, the mediator(s) contained in the third nonaqueous phase acts as reducing agent(s) or coenzyme(s) in the reduction reaction of generating the third reaction product.

The enzyme body(s) contained in the first nonaqueous phase may include enzyme body(s) which catalyzes a reduction reaction of oxidant(s) of the mediator(s). Such enzyme body(s) may include enzyme(s) which catalyzes the reduction reaction of oxidant(s) of the mediator(s). The enzyme body which catalyzes the reduction reaction of reducing carbon dioxide to the first reaction product and the enzyme body which catalyzes the reduction reaction of oxidant(s) of the mediator(s) may be either the same enzyme body or different enzyme bodies.

The enzyme body(s) contained in the second nonaqueous phase may include enzyme body(s) which catalyzes a reduction reaction of oxidant(s) of the mediator(s). Such enzyme body(s) may include enzyme(s) which catalyzes the reduction reaction of oxidant(s) of the mediator(s). The enzyme body which catalyzes the reduction reaction of reducing the first reaction product to the second reaction product and the enzyme body which catalyzes the reduction reaction of oxidant(s) of the mediator(s) may be either the same enzyme body or different enzyme bodies.

The enzyme body(s) contained in the third nonaqueous phase may include enzyme body(s) which catalyzes a reduction reaction of oxidant(s) of the mediator(s). Such enzyme body(s) may include enzyme(s) which catalyzes the reduction reaction of oxidant(s) of the mediator(s). The enzyme body which catalyzes the reduction reaction of reducing the second reaction product to the third reaction product and the enzyme body which catalyzes the reduction reaction of oxidant(s) of the mediator(s) may be either the same enzyme body or different enzyme bodies.

In this mode, the first reaction product produced in the first nonaqueous phase is selectively extracted by an extraction liquid contained in the first aqueous phase. Therefore, the equilibrium of the reduction reaction of carbon dioxide in the first nonaqueous phase can be shifted to the first reaction product side. Also, in the second aqueous phase, the first reaction product as a substrate is supplied from the first aqueous phase, and the produced second reaction product is selectively extracted by an extraction liquid contained in the second aqueous phase. Accordingly, the equilibrium of the reduction reaction of the first reaction product can be shifted to the second reaction product side. In the third nonaqueous phase, the second reaction product as a substrate is supplied from the second aqueous phase, and the produced third reaction product is selectively extracted by an extraction liquid contained in the third aqueous phase, and recovered. Accordingly, the equilibrium of the reduction reaction of the second reaction product can be shifted to the third reaction product side.

As described above, the equilibrium of the three-cascade reaction of reducing carbon dioxide to the third reaction product as a final product can be shifted toward the final product side at each reaction cascade, so the final product can be recovered at high yield. Also, a high-purity final product can be obtained because the reactant (carbon dioxide) and the intermediate products (first and second reaction products) hardly mix in the final product (third reaction product). Furthermore, in each of the cascades of generating the intermediate products in the first and second nonaqueous phases, the first and second aqueous phases respectively recover the intermediate products, i.e., the first and second reaction products. This suppresses the loss of the intermediate products.

In an exemplary modification of the first mode, a two-cascade enzymatic reaction of reducing carbon dioxide to a final product in two cascades may be adopted instead of the three-cascade enzymatic reaction. In this case, the nonaqueous phase includes first and second nonaqueous phases. In the first nonaqueous phase, carbon dioxide is supplied, and a reduction reaction of this carbon dioxide generates a first reaction product. In the second nonaqueous phase, the first reaction product is supplied, and a reduction reaction of this first reaction product generates a second reaction product. The second reaction product generated in the second nonaqueous phase is supplied to the aqueous phase.

In this modification, only the first reaction product is an intermediate product. Also, the second reaction product is a final product. Therefore, the second reaction product is extracted by an extraction liquid contained in a second aqueous phase, and recovered. The arrangement of this modification is the same as that of the first mode except this point, so an explanation thereof will be omitted.

The reaction of reducing carbon dioxide may alternatively be a cascade reaction including four or more cascades, as needed. The numbers of nonaqueous phases and aqueous phases may appropriately be changed in accordance with the number of reaction cascades included in the cascade reaction. Furthermore, each nonaqueous phase may also contain a substrate other than carbon dioxide and an intermediate product (a reduced product of carbon dioxide), as needed.

The carbon dioxide fixation device of the first mode may take either a flow-type form or batch-type form.

In the flow-type form, each of the supply of carbon dioxide to the first nonaqueous phase, the supply of a reaction product from each nonaqueous phase to the next aqueous phase, and the supply of a reaction product from each aqueous phase to the next nonaqueous phase is continuously performed.

In the batch-type form, at least the supply of a reaction product from each aqueous phase to the next nonaqueous phase is discontinuously performed. When a three-cascade reaction is adopted, for example, carbon dioxide supplied to the first nonaqueous phase is reduced to generate a first reaction product, the first reaction product is supplied to the first aqueous phase, and thereafter, the first reaction product thus recovered by the first aqueous phase is temporarily stored there. Likewise, the second reaction product generated in the second nonaqueous phase is supplied to the second aqueous phase, and temporarily stored in the second aqueous phase. This batch-type form may be obtained by installing, for example, a mechanism which controls the flow of an extraction liquid in each of the first and second aqueous phases.

In the second mode of the carbon dioxide fixation device according to the embodiment, the nonaqueous phase may include first, second, and third nonaqueous phases. That is, the carbon dioxide fixation device of this mode may include a plurality of nonaqueous phases. Carbon dioxide is supplied to the first nonaqueous phase. In the first nonaqueous phase, a reduction reaction of the supplied carbon dioxide generates a first reaction product. The first reaction product is supplied to the second nonaqueous phase. In the second nonaqueous phase, a reduction reaction of the supplied first reaction product generates a second reaction product. The second reaction product is supplied to the third nonaqueous phase. In the third nonaqueous phase, a reduction reaction of the supplied second reaction product generates a third reaction product. The third reaction product generated in the third nonaqueous phase is supplied to the aqueous phase.

In this mode, the three-cascade enzymatic reaction in the first to third nonaqueous phases reduces carbon dioxide to a final product (fuel). That is, the first and second reaction products are intermediate products of the three-cascade reaction, and the third reaction product is the final product.

Each of the first to third nonaqueous phases may contain one or more kinds of ionic liquids, one or more kinds of enzyme bodies, and one or more kinds of mediators. The kinds and numbers of the ionic liquids, enzyme bodies, and mediators contained in the first to third nonaqueous phases may be different from each other, or may alternatively be the same.

The enzyme body(s) contained in the first nonaqueous phase contains enzyme(s) which catalyzes a reduction reaction of generating a first reaction product by reducing carbon dioxide. Also, the mediator(s) contained in the first nonaqueous phase acts as reducing(s) agent or coenzyme(s) in the reduction reaction of generating the first reaction product.

The enzyme body(s) contained in the second nonaqueous phase contains enzyme(s) which catalyzes a reduction reaction of generating a second reaction product by reducing the first reaction product. Also, the mediator(s) contained in the second nonaqueous phase acts as reducing agent(s) or coenzyme(s) in the reduction reaction of generating the second reaction product.

Likewise, the enzyme body(s) contained in the third nonaqueous phase contains enzyme(s) which catalyzes a reduction reaction of generating a third reaction product by reducing the second reaction product. Also, the mediator(s) contained in the third nonaqueous phase acts as reducing agent(s) or coenzyme(s) in the reduction reaction of generating the third reaction product.

The enzyme body(s) contained in the first nonaqueous phase may include enzyme body(s) which catalyzes a reduction reaction of oxidant(s) of the mediator(s). Such enzyme body(s) may contain enzyme(s) which catalyzes the reduction reaction of oxidant(s) of the mediator(s). The enzyme body which catalyzes the reduction reaction of reducing carbon dioxide to the first reaction product and the enzyme body which catalyzes the reduction reaction of oxidant(s) of the mediator(s) may be either the same enzyme body or different enzyme bodies.

The enzyme body(s) contained in the second nonaqueous phase may include enzyme body(s) which catalyzes a reduction reaction of oxidant(s) of the mediator(s). Such enzyme body(s) may contain enzyme(s) which catalyzes the reduction reaction of oxidant(s) of the mediator(s). The enzyme body which catalyzes the reduction reaction of reducing the first reaction product to the second reaction product and the enzyme body which catalyzes the reduction reaction of oxidant(s) of the mediator(s) may be either the same enzyme body or different enzyme bodies.

The enzyme body(s) contained in the third nonaqueous phase may include enzyme body(s) which catalyzes a reduction reaction of oxidant(s) of the mediator(s). Such enzyme body(s) may contain an enzyme which catalyzes the reduction reaction of oxidant(s) of the mediator(s). The enzyme body which catalyzes the reduction reaction of reducing the second reaction product to the third reaction product and the enzyme body which catalyzes the reduction reaction of oxidant(s) of the mediator(s) may be either the same enzyme body or different enzyme bodies.

In the carbon dioxide fixation device of the second mode, the first reaction product generated in the first nonaqueous phase is directly supplied to the second nonaqueous phase. Also, the second reaction product generated in the second nonaqueous phase is directly supplied to the third nonaqueous phase.

As described above, when supplying the intermediate products (first and second reaction products) to the nonaqueous phases (second and third nonaqueous phases) including the reaction fields of the next reaction cascades in the cascade reaction of reducing carbon dioxide, these intermediate products are supplied without being passed through any aqueous phases. This makes it possible to reduce the loss of the intermediate products.

In an exemplary modification of the second mode, a two-cascade enzymatic reaction of reducing carbon dioxide to a final product may be adopted instead of the three-cascade enzymatic reaction. In this case, the nonaqueous phase includes first and second nonaqueous phases. In the first nonaqueous phase, carbon dioxide is supplied, and a reduction reaction of this carbon dioxide generates a first reaction product. In the second nonaqueous phase, the first reaction product is supplied, and a reduction reaction of this first reaction product generates a second reaction product. The second reaction product generated in the second nonaqueous phase is supplied to the aqueous phase.

In this modification, only the first reaction product is an intermediate product. Also, the second reaction product is a final product. Therefore, the second reaction product is extracted by an extraction liquid contained in the second aqueous phase, and recovered. The arrangement of this modification is the same as that of the second mode except this point, so an explanation thereof will be omitted.

As in the first mode, the reaction of reducing carbon dioxide may alternatively be a cascade reaction including four or more cascades, as needed. The numbers of nonaqueous phases and aqueous phases may appropriately be changed in accordance with the number of reaction cascades included in the cascade reaction. Furthermore, each nonaqueous phase may contain a substrate other than carbon dioxide and intermediate products (a reduced product of carbon dioxide) as needed.

Next, details of the nonaqueous phase and aqueous phase in the carbon dioxide fixation device according to the embodiment will be explained.

<Nonaqueous Phase>

The nonaqueous phase contains an ionic liquid, enzyme body, and mediator.

[Ionic Liquid]

As a medium of the nonaqueous phase, it is desirable to use a material having the following properties:

(1) The ability to absorb and separate CO₂ is high.
(2) The vapor pressure is low.
(3) The thermal stability is high.
(4) The electrical conductivity is high.
(5) The potential window is wide.
(6) The chemical stability is high.
(7) Usable as a stabilizer of protein.

As long as the material is a nonaqueous phase material having the above primary properties, the material is not particularly limited, but it is desirable to use an ionic liquid as a medium of the nonaqueous phase. The ionic liquid has a low vapor pressure (close to zero), has a wide potential window, sufficiently absorbs a specific substance, e.g., carbon dioxide, has a high thermal stability, and has a high electrical conductivity. Therefore, it is desirable to use the ionic liquid as a medium of the nonaqueous phase.

An ionic liquid having a high CO₂ solubility can absorb a large amount of carbon dioxide. For example, when using such an ionic liquid as a medium of the nonaqueous phase, CO₂ as a reactant can efficiently be supplied to the nonaqueous phase containing enzyme body(s) which serves as the field(s) of CO₂ reduction reaction. When using the ionic liquid as a medium of the nonaqueous phase, the CO₂ absorption amount increases as the pressure increases. For example, it is desirable to use an ionic liquid capable of absorbing 1% (CO₂ mole % in ionic liquid) or more of CO₂ when the pressure is increased to 0.1 MPa, and 60% (CO₂ mole % in ionic liquid) or more of CO₂ when the pressure is increased to 6 MPa.

As described above, the ionic liquid has a high electrical conductivity, and is excellent in both ion conductivity and electron conductivity. Since a high ion conductivity and high electron conductivity are advantageous for electron migration in the nonaqueous phase, it is possible to rapidly and efficiently generate a product (e.g., fuel). For example, when the mediator(s) acts as reducing agent(s) or coenzyme(s) in the enzymatic reaction of the enzyme body, electrons easily transfer from the mediator(s) to the oxidation/reduction site(s) of the enzyme(s), and this accelerates the enzymatic reaction(s). Also, when regenerating or reducing the mediator(s) by the action of an electrode as will be described later, electron transfer from the electrode to the mediator(s) is accelerated, so the regeneration and reduction can be performed efficiently.

The ionic liquid is also called a designer liquid, and the properties of the ionic liquid can freely be designed by properly selecting an anion and cation for the ionic liquid in accordance with use.

Examples of typical cations of the ionic liquid include imidazolium, ammonium, phosphonium, and pyridinium.

Examples of other typical cations of the ionic liquid include 1-methyl-3-alkylimidazolium, 1,3-bis[3-methylimidazolium-1-yl]alkane, poly(diallyldimethylammonium), metal(M⁺) tetraglyme, and pyrrolidinium.

Examples of typical anions of the ionic liquid include bis(trifluoromethanesulfonyl)imide ([Tf₂N]⁻, [TFSI]⁻, or [TFSA]⁻), tetrafluoroborate ([BF₄]⁻), hexafluorophosphate ([PF₆]⁻), and dicyanoamine ([DCA]⁻).

Examples of other typical anions of the ionic liquid include halides, formate, nitrate, hydrogen sulfate, heptafluorobutyrate, thiocyanate, tris(pentafluoroethyl)trifluorophosphate, dicyanamide, poly(phosphonic acid), tetrachloroferrate, and trifluoromethanesulfonate ([TfO]⁻).

The ionic liquid can roughly be classified into a nonpolar ionic liquid and polar ionic liquid. Generally, the nonpolar ionic liquid is a hydrophobic ionic liquid, and the polar ionic liquid is a hydrophilic ionic liquid.

The hydrophilic polar ionic liquid can further be classified into a protic ionic liquid and aprotic ionic liquid. The polar ionic liquid can also be used as a core solution for a reversed micelle formed by a hydrophilic solvent. In addition, the protic ionic liquid can be made into a gel (membrane) and used as a PEM (Proton Exchange Membrane), in a similar manner as Nafion®. The aprotic ionic liquid can be used as a refolding agent and heat stabilizer of protein (enzyme).

As described above, the ionic liquids can selectively be used in accordance with their properties.

Of ionic liquids, the use of an imidazole-based ionic liquid is desirable. The imidazole-based ionic liquid or its gel can absorb CO₂ with high selectivity, and has high electrical conductivity.

Of the hydrophobic ionic liquids, the use of imidazole-based 1-ethyl-3-methylimidazolium bis(fluorosulfonyl)imide (EMIMBTI) having high absorbency with regard to CO₂ is particularly desirable.

Many names for EMIMBTI including an official name, other names, and abbreviations exist. For example, EMIMBTI is also called 1-ethyl-3-methylimidazolium bis(trifluoromethylsulfonyl)amide, 1-ethyl-3-methylimidazolium bis(trifluoromethylsulfonyl)imide, and 1-ethyl-3-methylimidazolium bis(trifluoromethanesulfonyl)imide. Examples of the abbreviations are EMIMIm, [emim][(CF₃SO₂)₂N], [emim][TFSI], EmimTFSI, EMIMTFSI, EmimNTf₂, EmimTf₂N, Emim TFSI, Emim NTf₂, Emim Tf₂N, [C₂mim][NTf₂], BMIM-NTf₂, [C₂mIm⁺][TFSI⁻], and [C₂C₁im][NTf₂]. To avoid confusion, the molecular structure is presented below:

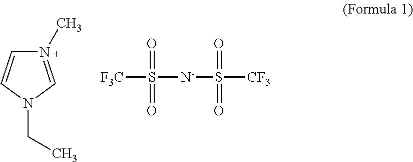

Two or more kinds of ionic liquids may be used in the form of a mixture, taking account of the absorbency to CO₂ and properties such as the cost, toxicity, viscosity, and conductivity. For example, it is possible to reduce the material cost and reduce toxin by mixing an ionic liquid having high absorbency with regard to CO₂, and an ionic liquid having toxicity and cost that are lower than those of the former ionic liquid.

Also, among polar ionic liquids, an aprotic ionic liquid can be used as a heat stabilizer for protein. Therefore, the polar ionic liquid can be mixed with the nonpolar ionic liquid and used as a nonaqueous phase.

Furthermore, the cathodic limit of the ionic liquid depends on the properties of the cation, and extends if the cation contains an electron-withdrawing group. Accordingly, it is desirable to select the ionic liquid from ionic liquids which sufficiently absorb carbon dioxide and contain a cation containing an electron-withdrawing group.

As the ionic liquid, it is further possible to use, e.g., 1-methyl-3-propyl-methylimidazolium dihydrogen phosphate (PMIH₂PO₄), polybenzimidazole (PBI), 1-octyl-3-methylimidazolium bis(trifluoromethanesulfonyl)amide, [C₈mIm⁺][TFSA⁻] (TFSA⁻=(CF₃SO₂)₂N⁻), 1-alkylimidazolium bis(trifluoromethanesulfonyl)amide, [CₙImH⁺][TFSA⁻] (n=4 or 8), triethyl sulfonium bis(trifluoromethyl sulfonyl)imide (TSBTSI), 1-butyl-3-methylimidazolium hexafluorophosphate ([bmim][PF₆]), 1-butyl-2,3-dimethylimidazolium bis(trifluoromethylsulfonyl)imide ([bmim][Tf₂N]), octyl-3-methylimidazoliumhexafluorophosphate ([omim][PF₆]), 1-decyl-3-methylimidazolium bis(trifluoro-methylsulfonyl)imide ([dmim][Tf₂N]), 1-butyl-3-methylimidazolium tetrafluoroborate ([bmim][BF₄]), 1-dodecyl-3-methylimidazolium chloride ([dmim][Cl]), 1-methyl-3-octylimidazolium chloride (MOImCl), [C₂mim][NTf₂], [C₆mim][NTf₂], [C₈mim][NTf₂], [C₆F₉MIM][NTf₂], 1-butyl-3-methylimidazolium bis[(trifluoromethyl)sulfonyl]imide, [C₄mim][NTf₂], [C₄mim][PF₆], [Bmim][PF₆] (BMIM-PF₆), [bmmim][PF₆], [C₄mim][TFSI], IL[C₉mim][Tf₂N] (1-octyl-3-methylimidazolium bis(trifluoromethylsulfonyl)amide), IL₂(1-ethyl-3-methyl-imidazolium bromide, [emim][Br]), 1-ethyl-3-methylimidazolium trifluoromethanesulfonate ([emim][Tf]), 1-ethyl-3-methylimidazolium tetra-fluoroborate ([emim][BF₄]), [emim][AlCl₄], [emim][H_{2,3}F_{3,3}], [emim][CH₃CO₂], [emim][CF₃SO₃], [emim][(C₂F₅SO₂)₂N], [emim][(C₂F₅SO₂)₂C], [emim][TFA], [emim][Ac], 1-butyl-3-methylpyridinium tetrafluoroborate ([bmpyri][BF₄]), 1-butyl-3-methylpyrrolidinium tetrafluoroborate ([bmpyrro][BF₄]), [bmim][BF₄], 1-ethyl-3-methylimidazolium chloride ([emim][Cl]), [emim][Tf₂N], [C₄mim][AC], [P₆₆₁₄][Pro], [P₆₆₁₄][Ala], [P₆₆₁₄][Gly], [MTBDH][TFE], [C₂mim][DCA], [C₂mim][Tf₂N], [C₂mim][TfO], and [C₂mim][TCB].

Other ionic liquid that may be used include, e.g., N-(2-methoxyethyl-N-methylpyrrolidinium tetrafluoroborate (MEMPBF₄), N-(2-methoxyethyl-N-methylpyrrolidinium bis(trifluoromethanesulfonyl)imide (MEMPTFSI), N,N-diethyl-N-methyl-N-(2-methoxyethyl)ammonium tetrafluoroborate (DEMEBF₄), N,N-diethyl-N-methyl-N-(2-methoxyethyl)ammonium bis(trifluoromethanesulfonyl)imide (DEMETFSI), N,N,N-trimethyl-N-propylammonium bis(trifluoromethanesulfonyl)imide (TMPA TFSI), N-methyl-N-propylpiperidinium bis(trifluoromethanesulfonyl)imide (PP13 TFSI).

Like EMIMBTI, a particular of the ionic liquids described above may have the same molecular structure although their expressions and signs are different. Also, a new ionic liquid may be synthesized as needed in addition to the above ionic liquids.

Furthermore, it is known that physical absorption of CO₂ by an imidazole ([Emim]⁺)-based ionic liquid, has dependency on pressure.

For example, in [emim][TFSA], [emim][BETA], [emim][NFBS], and [emim][BF₄] as examples of the imidazole-based ionic liquid, the solubility of CO₂ in the ionic liquid is on the order of 0.1 mole fraction or less to 0.2 mole fraction or less at 40° C. (313K) and standard atmospheric pressure (0.101325 MPa=1 atm). On the other hand, these ionic liquids exhibit a CO₂ solubility on the order of 0.3 mole fraction or more to 0.6 mole fraction or more at 40° C. (313K) under the pressure condition of 6 MPa (=60 atm). When the pressure is increased from standard atmospheric pressure to 6 MPa at 40° C. (313K), for example, the CO₂ solubility in the ionic liquid can largely be increased (e.g., three to four times), although it depends on the kind of the imidazole-based ionic liquid.

As described above, when the temperature is constant, the efficiency of physical absorption by the ionic liquid increases as the pressure of supplied CO₂ rises.

The gas-liquid critical point of CO₂ is at a temperature of 31° C. and a pressure of 7.4 MPa. When supplying CO₂ in the form of a gas to the ionic liquid used as a medium of the nonaqueous phase, CO₂ is preferably supplied under the conditions in which CO₂ does not become a supercritical fluid. When the temperature or pressure rises beyond the gas-liquid critical point, CO₂ may become a supercritical fluid. For example, under conditions where the temperature is 31° C., CO₂ can be supplied as a gas to the nonaqueous phase when the pressure is 7.4 MPa or less.

On the other hand, the solubility of CO₂ in the ionic liquid depends on the temperature as well. Generally, the solubility of CO₂ in the ionic liquid decreases as the temperature rises. Accordingly, it is desirable to set an optimal temperature in accordance with the properties of activity for an enzyme at each temperature.

The solubility of CO₂ in the ionic liquid is also influenced by the cation species and anion species. Regarding CO₂ solubility in ionic liquids, there can be seen a trend where anion species has influence larger than that of cation species.

As a practical example, ionic liquids having the same Bmim cation absorb more CO₂ according to the following anion order:

NO₃⁻<DCA⁻<BF₄⁻<PF₆⁻<TfO<TFSA

Ionic liquids having the same Emim cation absorb more CO₂ according to the following anion order:

BF₄⁻<NFBS<TFSA<BETA

[Emim][PF₆] exhibits CO₂ absorptivity that is approximately the same as that of [Emim][BF₆]. On the other hand, CO₂ absorptivity of [Emim][DCA] is lower as compared to those of [Emim][BF₆] and [Emim][PF₆].

One reason why the CO₂ solubility in the ionic liquid depends on the cation species and anion species of the ionic liquid is the existence of unoccupied spaces dispersed within the ionic liquid. Unoccupied spaces regularly dispersed within the ionic liquid are used as space for accommodating CO₂ molecules. When CO₂ is absorbed into these unoccupied spaces, the unoccupied spaces widens (expands) in accordance with the displacement of cations and anions in the ionic liquid.

The ratio of the unoccupied space in the ionic liquid tends to increase in accord with decrease in the force (cohesive force) by which cations and anions attract each other in the ionic liquid. The greater the unoccupied space(s) in the ionic liquid, the higher the CO₂ absorption property.

Many imidazole-based ionic liquids physically absorb CO₂ based on the above-described principle.

In the embodiment, therefore, based on the CO₂ absorption property of ionic liquids having the same Emim cation, it is possible to use different ionic liquids by appropriately mixing them, as needed.

Also, in ionic liquids having the same Emim cation, the longer the perfluoroalkyl chain, the larger the effect of improving the CO₂ solubility. By taking account of this effect as well, therefore, it is favorable to appropriately design and synthesize the perfluoroalkyl chain of the ionic liquid.

As the ionic liquid for physically absorbing carbon dioxide, it is possible to use, e.g., [C₄mim][Nf₂T], [C₄mim][PF₆], [C₄mim][BF₄], [C₈mim][Nf₂T], and [C₂mim][eFAP].

On the other hand, chemical absorption of CO₂ by the ionic liquid can be performed by using an amine-based ionic liquid or amino acid-based ionic liquid.

As the ionic liquid for chemically absorbing carbon dioxide, it is possible to use, e.g., [P₆₆₁₄][Pro], [P₆₆₁₄][Ala], [P₆₆₁₄][Gly], [MTBDH][TFE], [N₁₁₁₄][Tf₂N], and [MTBDH][Im].

A hydrophobic ionic liquid is not largely influenced by water because the solubility of water in such an ionic liquid is low. Also, the use of an ionic liquid achieves the following advantages regardless of whether the ionic liquid is hydrophobic or hydrophilic:

1) Volume expansion caused by gas absorption is small.
2) The ionic liquid is chemically stable over a broad temperature range.
3) Risks of accidents and the like are small because the ionic liquid is nonflammable.

To improve the CO₂ isolating effect or to prevent problems such as leakage and outflow of the ionic liquid, it is further desirable to immobilize the ionic liquid with an immobilization support. For example, the ionic liquid can be immobilized by forming an ionic liquid membrane on a support material.

As the ionic liquid immobilization support, it is possible to use, e.g., a porous membrane, porous monolayer material, zeolite, silica glass membrane, and polymer.

When using a polymer as a support for the ionic liquid, it is possible to form polymer-ionic liquid composite materials such as a polymer-ionic liquid composite membrane and polymer-ionic liquid fiber by casting, electronic spinning, or suction. As the polymer, it is possible to use, e.g., poly(vinylidene difluoride) (PVDF) or poly(vinylidene difluoride)-hexafluoropropylene (PVDF-HFP).

Also, a polymer-ionic liquid membrane can be formed on the surface of an electrode by dissolving a polymeric material and ionic liquid material in a solvent, and spraying the solution on a porous electrode material. Since the ionic liquid can be immobilized in the form of a membrane or fiber, it is possible to prevent leakage of the ionic liquid caused by CO₂ absorption resulting from pressurization.

As the support material for forming the ionic liquid membrane, it is possible to use, e.g., a hydrophilic or hydrophobic polymeric material. As the hydrophobic polymeric material, polyvinylidene fluoride (PVDF) or the like may be used. As the hydrophilic polymeric material, it is possible to use, e.g., polyethylene (PE), polytetrafluoroethylene (PTFE), or polyvinylalcohol (PVA). Alternatively, the hydrophobic and hydrophilic polymeric materials may be used as a mixture. When mixing the polymeric materials, it is possible to use, e.g., PVDF and PTFE in the form of a mixture.

Other than the polymeric material, it is also possible to use, e.g., an anodic oxidation alumina nanoporous membrane, porous TiO₂, or mesoporous carbon, as the support material which makes the ionic liquid usable as a membrane.

The ionic liquid may also be gelled by a gelling agent, thereby forming an ion gel, which is also called ionogel. An ion gel membrane can be formed by further forming a membrane of the ion gel. As the gelling agent, it is possible to use, e.g., gelatin, MOGs, or hydroxy propyl methylcellulose (HPMC).

The ionic liquid may also be used as a polymerized ionic liquid, by undergoing polymerization. A poly(ionic liquid) membrane can be formed by further forming a membrane of the polymerized ionic liquid.

In addition, a porous ionic liquid structure having a porous structure may be used as the ionic liquid. The porous ionic liquid structure can be formed by, e.g., cross-linking the ionic liquid in the presence of a mold made of TiO₂ or the like, and subsequently removing the mold (e.g., etching or displacement removal). The porous ionic liquid structure can also be formed under mold-free conditions. As the porous ionic liquid structure usable in the embodiment, a porous ionic liquid structure capable of sufficiently absorbing CO₂ is desirable, and the type of the structure is not particularly limited.

When using the ionic liquid as a medium of the nonaqueous phase, it is desirable to form a permeation-separation membrane (permselective membrane) (e.g., separator described later) between the nonaqueous phase containing the ionic liquid and the aqueous phase, in order to prevent an outflow of the ionic liquid to the aqueous phase.

[Enzyme Body]

The enzyme body, which is included in the nonaqueous phase, includes one or more kinds of enzymes. The enzyme body may be a lone enzyme. Alternatively, the enzyme body may include an immobilized enzyme. Enzyme immobilization herein mentioned includes bonding an enzyme to a support by a support bonding method, entrapping an enzyme in a polymer gel or microcapsule by an entrapping method, and bonding enzymes to one another by a crosslinking method. The enzyme body obtained by immobilizing the enzyme includes, e.g., a composite including a molecular aggregate formed by a dispersant and the enzyme, a microcapsule encapsulating the enzyme, and a composite including a support formed by a polymeric material or the like and the enzyme supported on or contained in the support. A biological cell or microorganism including the enzyme may also be used as the enzyme body.

An enzymatic reaction requires water in most cases. This is so because an enzyme is originally a biocatalyst which functions in water. An enzyme normally shows a high enzyme activity in water because the enzyme becomes flexible in water. By contrast, the activity of an enzyme significantly decreases in a waterless system. Therefore, in an enzymatic reaction system in a nonaqueous phase, presence of an appropriate amount of water is desirable. Although the form of presence of water depends on the form of the enzyme body, as long as high enzyme activity can be obtained, the included amount and form of presence of the water is not particularly limited.

The enzyme body may include water, and this water can function as an enzymatic reaction field of the enzyme. Therefore, the enzyme shows a high enzyme activity in the enzyme body including water.

The enzyme bodies are dispersed in the medium in the nonaqueous phase.

The nonaqueous phase may include one type of enzyme bodies where each enzyme body includes two or more kinds of enzymes. Alternatively, the nonaqueous phase may include plural types of enzyme bodies each including different kinds of enzymes. In this case, each enzyme body may include only one kind of enzyme, or may include two or more kinds of enzymes.

When the nonaqueous phase includes plural types of enzyme bodies including different kinds of enzymes, a part of a reaction product formed by an enzymatic reaction in one enzyme body may function as a substrate of an enzymatic reaction in another enzyme body. Chemical substances are rapidly exchanged between individual enzyme bodies included within the same system. Therefore, the reaction product formed by the enzymatic reaction in one enzyme body rapidly transfers to another enzyme body and participates in the enzymatic reaction there as a substrate.

By performing the enzymatic reaction in the nonaqueous solvent, it is also possible to prevent propagation of viruses, fungi, bacteria, algae, and the like. The enzymatic reaction is desirably performed in the enzyme body dispersed within a medium in the nonaqueous phase from this viewpoint as well.

Water required to achieve the activity of the enzyme is referred to as “essential water”. That is, “essential water” desirably exists around an enzyme dispersed within the nonaqueous phase. For example, when dispersing a hydrophilic enzyme in the nonaqueous phase, a thin layer of water formed by about 50 to 500 water molecules per enzyme desirably exists as the “essential water” around the enzyme.

Note that the use of an enzyme body obtained by immobilizing an enzyme is desirable because the enzyme body achieves the following properties:

(1) A high-concentration of substrate (e.g., carbon dioxide or its reduced product) can be used.
(2) The stress resistance of the enzyme improves.
(3) The enzyme (enzyme body) and a product are easily separated.
(4) A repetitive use of the enzyme is facilitated.

(Enzyme)

An enzyme which may be included in an enzyme body is not particularly limited as long as the enzyme catalyzes a reaction of reducing carbon dioxide and generating fuel when used singly or in combination with other enzymes. Examples of a usable enzyme are an enzyme which functions as a catalyst of an enzymatic reaction of reducing carbon dioxide and generating an intermediate product, an enzyme which functions as a catalyst of an enzymatic reaction using, as a substrate, a reaction product of carbon dioxide (e.g., a reduced product of carbon dioxide as the above-described intermediate product), an enzyme which captures carbon dioxide, and an enzyme which reduces NAD⁺ to NADH.

Practical examples of the enzyme include oxidoreductase-dehydrogenases such as formate dehydrogenase (FDH), metal-dependent formate dehydrogenase, formaldehyde dehydrogenase, alcohol dehydrogenase, carbonic anhydrase, glutamate dehydrogenase (GDH), and molybdenum-containing formate dehydrogenase (Mo-FDH). Note that GDH is an example of the enzyme which reduces NAD⁺ to NADH.

As described later, NAD⁺ and NADH are respectively, an oxidized form (i.e., an oxidant) and a reduced form (i.e., a reductant) of nicotinamide adenine dinucleotide, which is a mediator. Although GDH was exemplified as a practical example of an enzyme that reduces NAD⁺ to NADH, other enzymes may alternatively be used. For example, PTDH can be noted as another enzyme that reduces NAD⁺ to NADH. Further, although NADH/NAD⁺ was exemplified as one example of a mediator that can be regenerated by an enzyme, there is of course no limitation to the mediator, which can be regenerated by the enzyme that may be included in an enzyme body. The enzyme bodies may include various kinds of enzymes that can reduce various oxidized mediators.

Other examples include a tungsten-containing formate dehydrogenase enzyme (FDH1), carbon monoxide dehydrogenase (CODH), and remodeled nitrogenase. Remodeled nitrogenase is an enzyme which reduces carbon dioxide to methane.

In addition to the above enzymes, it is also possible to use an enzyme which converts, by a cascade enzymatic reaction, carbon dioxide into useful materials having an added value higher, other than fuel.

Some species of enzymes can exhibit catalytic activity even at a high pressure. For example, there is an enzyme which can function even at a pressure of 200 MPa. Each of different species of enzymes has a pressure at which the reaction rate of a reaction which the enzyme catalyzes becomes maximum under predetermined conditions (other than pressure), i.e., each enzyme has an optimal pressure. For example, under predetermined conditions (other than pressure), the reaction rate increases as the pressure is increased, and becomes maximum when the optimal pressure is reached. The reaction rate decreases at a pressure higher than the optimal pressure. For example, an enzyme generally denatures at a pressure of about 400 MPa or more and may become unable to maintain function.

As described earlier, the CO₂ solubility of the ionic liquid improves as the pressure rises. Therefore, it is favorable to use an enzyme which shows catalytic activity even under conditions where the pressure is raised in order to promote CO₂ absorption to the nonaqueous solvent. More specifically, an enzyme contained in the enzyme body is preferably an enzyme which maintains the function of catalyzing a reduction reaction of CO₂ or a reduced product of CO₂ even at a pressure of 400 MPa. For example, it is more preferable to use an enzyme which sustains activity within the range of 0.101325 MPa (standard pressure) to 7.4 MPa at a temperature of standard temperature (298 K) to 304 K.

(Dispersant)

An emulsifying agent may be used as the dispersant. An emulsifying agent is an amphipathic molecule having a hydrophilic group and hydrophobic group. The kinds and combinations of emulsifying agents used in the embodiment are not particularly limited, as long as a stable molecular aggregate can be formed using the emulsifying agent. For example, a lipid, boundary lipid, sphingolipid, fluorescent lipid, a cationic surfactant, an anionic surfactant, an ampholytic surfactant, a zwitterionic surfactant, a nonionic surfactant, a sugar surfactant, a synthetic polymer, and a natural polymer such as protein may be selected as appropriate, to be used as the emulsifying agent.

As the zwitterionic surfactant, N-dodecyl-N, N-dimethyl-3-ammonio-1-propane sulfonate (SB-12) may be used, for example.

(Molecular Aggregate)

By using the dispersant in the nonaqueous phase, one or more molecular aggregate selected from a nearly spherical reversed micelle, a reverse wormlike micelle, liposome, vesicle, a microemulsion, a larger emulsion, a bicontinuous microemulsion, a monodispersed single emulsion, a double emulsion, and a multilayered emulsion may be formed.

The enzyme body may be obtained by immobilizing the enzyme to such a molecular aggregate. As an example of the molecular aggregate, a nearly spherical reversed micelle formed in the nonaqueous phase by the dispersant can maintain a predetermined amount of water in a central portion as the water pool. The enzyme may be immobilized by being entrapped in the water pool of the reversed micelle. Such immobilization of the enzyme is referred to as solubilization of the enzyme into the water pool. In the enzyme body, the water pool may be used as the field of the enzymatic reaction catalyzed by the enzyme.

The reversed micelle may be formed, for example, as follows. An emulsifying agent may be added to a medium of the nonaqueous phase. When the concentration of the emulsifying agent reaches a critical micelle concentration (CMC), a hydrophilic group and hydrophobic group of the emulsifying agent respectively face the inside and outside, thereby forming a nearly spherical reversed micelle surrounding water.

By further increasing the concentration of the emulsifying agent and thereby growing the spherical reversed micelle, a reverse wormlike micelle can be formed. Water within the interior of the reverse wormlike micelle may be the reaction field of the enzymatic reaction like that in the reversed micelle. Also, by using the reverse wormlike micelle as the enzyme body, a nonaqueous phase including the enzyme body can be gelled. Details of gelling the nonaqueous phase will be described later.

Reversed micelles or reverse wormlike micelles may also be produced, for example, by adding to a nonaqueous solvent, a surfactant such as sodium 1,2-bis(2-ethylhexylcarbonyl)-1-ethane sulfonate (Aerosol OT; AOT), instead of an emulsifying agent. Reverse wormlike micelles can be formed by increasing the concentration of AOT in the nonaqueous phase. When the AOT concentration is further increased, the reverse wormlike micelles become intertwined, and the whole medium of the nonaqueous phase becomes gelled.

As another molecular aggregate, for example, liposome, vesicle, a microemulsion, a larger emulsion, a bicontinuous microemulsion, a monodispersed single emulsion such as a water-in-oil type emulsion (W/O monodispersed emulsion), a double emulsion (W/O/W double emulsion), and a multilayered emulsion, formed by the dispersant may be used. These molecular aggregates may include an internal water phase or aqueous solvent as a water phase, which may be used as the water pool.

The aqueous solvent, which may be included in the molecular aggregate as the internal water phase or water phase, may include the above-described polar ionic liquid, for example. For example, a polar ionic liquid and an aqueous solvent may be mixed, and used as an internal phase (IL+W) of a mixed solvent made of the ionic liquid (IL) and the aqueous solution (W), such as for a (IL+W)/O microemulsion (water-in-oil type emulsion), reversed micelle [(IL+W)/O], or reverse wormlike micelles, which form in a nonpolar ionic liquid.

In the water pool, water that, exists in vicinity of a hydrophilic group of a molecule of a dispersant in a state where molecular motion is bound by ion-dipole interactions or a molecule of a hydrophilic group of a protonic ionic liquid (PIL) is called bound water. On the other hand, water existing in the central portion of the water pool is free water in almost the same state as that of bulk water. Exchange is rapidly performed between the free water and bound water. The amount of free water increases as a water content ωₒ increases. The water content ωₒ is obtained by the following equation.

ωₒ=[H₂O]/[S] (Equation 1)

Here, [H₂O] is the molar concentration of water, and [S] is the molar concentration of a dispersant (S).

Also, the radius (R_{w}) of the water pool is obtained by the following equation.

R_{w}=0.15ωₒ (Equation 2)

When using a protonic ionic liquid (PIL) as the ionic liquid, the PIL functions as a cosurfactant, and contributes to the formation of reversed micelles or a microemulsion (water-in-ionic liquid type; W/IL), as well. Therefore, it is necessary to take account of the amount of PIL used in the formation of reversed micelles or a microemulsion (W/IL). Generally, the water content ωₒ increases as the PIL amount increases when the concentration [S] of a surfactant is constant.

The size of the water pool can be appropriately adjusted by properly adjusting the water content ωₒ. The water content ωₒ may deviate from the above equation in cases where solvents such as polar ionic liquids also exist within the water pool, however.

The above-described molecular aggregate such as a reversed micelle, reverse wormlike micelle, liposome, vesicle, microemulsion, larger emulsion, W/O monodispersed emulsion, or W/O/W double emulsion may further be coated with a gel or polymeric material.

The molecular aggregate such as a reversed micelle, liposome, vesicle, microemulsion, larger emulsion, W/O monodispersed emulsion, or W/O/W double emulsion coated with a gel or polymer can be regarded as a microcapsule.

To increase the stability of the molecular aggregate or the efficiency of the enzymatic reaction, one or more types of materials selected from graphene oxide, carbon nanotubes, graphene, carbon nanohorns, silica nanoparticles, silver nanoparticles, gold nanoparticles, palladium nanoparticles, semiconductor nanoparticles, and mesoporous materials may be dispersed in the interior, on the surface, or in the periphery of the molecular aggregate. The interior of the molecular aggregate is, e.g., the water pool of a reversed micelle or the central core of a reverse wormlike micelle. Of these materials, when graphene oxide, carbon nanotubes, graphene, carbon nanohorns, silver nanoparticles, gold nanoparticles, or palladium nanoparticles are dispersed, a high electron conductivity, a high ion conductivity, and an effect of improving the stability of the molecular aggregate can be obtained. On the other hand, when silica nanoparticles, semiconductor nanoparticles, or a mesoporous material is dispersed, the effect of improving the stability of the molecular aggregate can be obtained.

In addition to the above-described dispersants, copper nanoparticles may be dispersed singly, or along with one or more described above. Also in the case of dispersing copper nanoparticles, electron conductivity and ion conductivity can be made high, and stability of the molecular aggregate can be improved.

In the case that one or more pairs of electrodes are disposed at the nonaqueous phase, as described later, precaution should be taken not to short-circuit the electrodes with one another, due to these materials. Short-circuiting of the electrodes may be prevented, for example, by dispersing the materials within the molecular aggregates, or arranging each of the electrodes sufficiently spaced apart. Furthermore, the size of the materials is preferably made small, in view of preventing short-circuiting of the electrodes.

(Microcapsule)

The microcapsule according to the embodiment refers to, for example a capsule obtained by encapsulating a core including a micronucleus (solid, liquid, or gas) with a porous membrane, and having a size ranging from a nanoscale to a millimeter scale. This microcapsule in the enzyme body has effects of, e.g., preventing alteration of the enzyme, and isolating, conserving, and hiding the enzyme from the nonaqueous solvent.

The above-described alteration of the enzyme includes, e.g., modification, denaturation, or the like of the enzyme.

The core of the microcapsule according to the embodiment may be used as the enzymatic reaction field. In addition, the microcapsule can rapidly entrap into the core, components which participate in the enzymatic reaction, such as a substrate, mediator, water, and intermediate product, and can also rapidly release the reaction product of the enzymatic reaction from the core.

As the membrane of the microcapsule, i.e., as the material of a shell, it is possible to use a hygroscopic polymeric material or another polymeric material, e.g. one that may be used as a support. That is, the membrane of the microcapsule may be one among an organic membrane made of a hygroscopic polymeric material or a polymeric material, an inorganic membrane, and an inorganic/organic hybrid membrane.

Generally, the microcapsule may be formed by any of three major methods, i.e., the chemical method, physicochemical method, and mechanical/physical method. Of these methods, examples of a method of forming a spherical mononuclear microcapsule include interfacial polymerization, in-situ polymerization, and in-liquid cured coating method as chemical methods, and in-liquid drying as a physicochemical method.

The microcapsule according to the embodiment may be formed by the above-described methods, and may also be formed by using as a template, a double emulsion formed, for example, by two-step emulsification, membrane emulsification, or one-step emulsification. A microcapsule obtained using a double emulsion formed by one-step emulsification as a template is particularly desirable because the amount of impurities in the core substance is small, there is small variation in the particle size, the number of cores, and the particle size of the core, and the enzyme can be encapsulated into the core while maintaining high activity.

The microcapsule may also be formed by photopolymerization of a reactive dispersant by using a reversed micelle, vesicle, or double emulsion formed by the dispersant.

The enzyme body may be a microcapsule that has an enzyme held therein. Such an enzyme body can be obtained, for example, by producing a microcapsule in such a manner that the enzyme would be encapsulated within the produced microcapsule, when producing the microcapsule by the above-described methods. The microcapsule may also hold therein a later-described cell or microorganism, instead of the enzyme. Before such obtained microcapsules (enzyme bodies) encapsulating enzyme(s) are dispersed in the nonaqueous phase, the microcapsules may be immersed in an aqueous solvent such that the core or membrane would include water.

(Cell and Microorganism)

A biological cell or microorganism including the enzyme may be used as the enzyme body. A cell or microorganism may singly be used as the enzyme body. Alternatively, a cell or microorganism immobilized by support bonding or entrapping may be used as the enzyme body.

The enzyme body may also be a cell or microorganism coated with a gel or polymeric material. Details of the gel or polymeric material for coating a cell or microorganism will be described later. When coating a cell or microorganism with a gel, extracellular matrix protein (ECM protein) or fibronectin (FN) as an extracellular matrix may also be used together with the gel to coat the cell or microorganism.

Cells and microorganisms existing in nature include various enzymes, and there exist cells and microorganisms having enzymes or combinations of enzymes useful for carbon dioxide fixation and conversion of carbon dioxide into fuel. A cell or microorganism having an appropriate combination of enzymes may be selected to be used as the enzyme body of the embodiment. Also, a cell that may be used for the embodiment may be a cell other than a microorganism, e.g., an animal cell or plant cell. In addition, a group of organisms generally referred to as algae, which use light such as solar light and artificial light as energy source, may be used in place of the above-mentioned microorganisms and cells.

A cell or microorganism may be used in a dead state where no reproduction occurs. Note that a microorganism in this dead state is referred to as being in a resting state. When such a microorganism in the resting state is immobilized, it is referred to as an immobilized resting cell.

(Support)

As the support for immobilizing the enzyme, for example, polysaccharides such as powder-form or porous bead-form chitin, chitosan (e.g., CHITO PEARL BCW3010® manufactured by FUJIBO), xylan, and K-carrageenan may be used. As the support, for example, porous glass, polylactic acid, alumina, silica gel, and celite may be used, also. In addition, for example, polysaccharide derivatives such as cellulose, dextran, and agarose may be used as the support.

The form of the support is not particularly limited, and may be in forms other than the above-described powder form and porous beads. For example, cellulose may be used in the form of a nonwoven, other than in the form of cellulose powder. Furthermore, as the support, for example, a cellulose powder, cellulose nanofiber (CNF), cellulose nanocrystal (CNC), chitin nanofiber, or chitosan nanofiber may be used. A typical CNF has a width of about 4 nm to 100 nm and a length of about 5 μm, and a typical CNC has a width of about 10 nm to 50 nm and a length of about 100 nm to 500 nm. Also, for example [BiNFi-s], which is a nanofiber derived from cellulose, chitin, and chitosan manufactured by SUGINO MACHINE, may be used. [BiNFi-s] has a diameter of about 20 nm and a length of a few μm.

The abovementioned support may be modified by the enzyme(s) by a support bonding method (physical adsorption method, ionic bonding method, or covalent bonding method), or the enzyme may be dispersed onto the support, thereby forming a composite. Alternatively, a lattice-form support, for example, may be modified with enzyme by an entrapment method (lattice type), and a composite may be formed by dispersing the enzyme within the network structure of the support. The composite obtained as such may be used as the enzyme body.

Not that, when a lattice-form support is modified with enzyme(s), the enzyme(s) may become entrapped within the lattice or mesh structure of the support.

A polymeric material may be used as the support. As polymeric material that may be used as the support, those made from a natural polymer or synthetic polymer are available.

As the natural polymer, for example starch-based polymers (e.g., starch-acrylonitrile graft polymer hydrolysate, starch-acrylate graft polymer, starch-styrene sulfonate graft polymer, starch-vinyl sulfonate graft polymer, and starch-acrylamide graft polymer), cellulose-based polymers (e.g., a cellulose-acrylonitrile graft polymer, a cellulose-styrene sulfonate graft polymer, and a crosslinked carboxymethylcellulose), other polysaccharide-based polymers (e.g., hyaluronic acid and agarose), and protein-based polymers (e.g., collagen) may be used.

A polymeric material made from a synthetic polymer is excellent in mechanical strength and chemical stability. As the synthetic polymer, for example polyvinyl alcohol-based polymers (e.g., a polyvinyl alcohol crosslinked polymer and PVA water-absorbing gel, elastomer), acryl-based polymers (e.g., a crosslinked sodium polyacrylate, sodium acrylate-vinyl alcohol copolymer, and polyacrylonitrile-based polymer saponified product), other addition polymers (e.g., a maleic anhydride-based polymer and vinyl pyrrolidone-based copolymer), polyether-based polymers (e.g., a polyethyleneglycol-diacrylate crosslinked polymer), and condensation polymers (an ester-based polymer and amide-based polymer) may be used.

The above-described polymer material may be processed into various forms such as a powder, bead, fiber, film, and nonwoven in accordance with usage.

With the aforementioned polymeric material as a support, the support may be modified with enzyme by the support bonding method (physical adsorption method, ionic bonding method, or covalent bonding method), thereby dispersing the enzyme onto the support and forming the enzyme body. Alternatively, a lattice-form support, for example, may be modified with enzyme by the entrapment method (lattice type), or the enzyme may be dispersed within the network structure of the support, thereby forming the enzyme body.

A polymer gel may also be used as the support for immobilizing an enzyme. As this gel, for example, Metrogel® (Metro Hydrogel®) made of a protein tropoelastin, gelatin methacrylate (GelMA) hydrogel, gelatin, alginate hydrogel, sodium polyacrylate gel, Mebiolgel® (manufactured by IKEDA KAGAKU), ambient temperature solidifying/elastic hydrogel AQUAJOINT® (manufactured by NISSAN CHEMICAL), silica gel, agar, κ-carrageenan, and polyacrylamide gel may be used.

The enzyme body may be produced by dispersing an enzyme onto the abovementioned gel or modifying the gel with enzyme by the bonding method (physical adsorption method, ionic bonding method, or covalent bonding method), or alternatively, by encapsulating the enzyme with the gel by the entrapment method.

Methods of separating and recovering CO₂ such as physical absorption/separation by an absorption solution or absorption material, separation by membrane separation, compressing and segregating CO₂ have various restrictions described below:

(1) The methods are applicable only to a low-pressure gas.
(2) The use of a corrosion-resistant vessel is necessary.
(3) Thermal energy is necessary.
(4) Separation of hydrogen gas is necessary.
(5) Energy for separating oxygen is necessary.
(6) Desulfurization is necessary.

Furthermore, a high overvoltage is necessary when converting CO₂ into fuel such as methanol by using the electrochemical conversion method. In addition, the electrochemical method also has problems such as producing a large amount of by-products.

By contrast, the method of converting CO₂ into fuel by using an enzyme does not have the above-described restrictions, and it is possible to use a lower overvoltage as Compared to when using an inorganic catalyst such as a metal catalyst. Therefore, converting CO₂ into fuel by using an enzyme is an attractive conversion method.

[Mediator]

The specie(s) of the mediator(s) according to the embodiment is not particularly limited as long as the mediator(s) is a substance which functions as mediator(s) of enzymatic reaction(s) which the enzyme(s) catalyzes. The mediator(s) itself may act as reducing agent(s) in the enzymatic reaction(s) of reducing a reactant, or may alternatively act as coenzyme(s) in the enzymatic reaction(s) of reducing a reactant.

For example, NADH (nicotinamide adenine dinucleotide) and PQQ (pyrroloquinoline quinone) are coenzymes and can be regarded as kinds of mediators.

A mediator is necessary in many enzymatic reactions for producing fuel from carbon dioxide. When using NADH as a mediator, NADH functions as a coenzyme.

In addition to nicotinamide adenine dinucleotide (NADH) (reductant)/nicotinamide adenine dinucleotide (NAD⁺) (oxidant) described above, it is also possible to use paired mediators such as NADPH/NADP⁺, MV⁺/MV⁺² (methylviologen), potassium ferricyanide/potassium ferrocyanide, hydroquinone/p-benzoquinone, pyrogallol/purpurogallin, and 3,3′,5,5′-tetramethylbenzidine (TMB)/3,3′,5,5′-tetramethylbenzidine diimine. Furthermore, in addition to pyrroloquinoline quinone (PQQ) described above, iodine, p-nitrophenol, phenol, aromatic amines, and the like may also be used as mediators.

Another further example of mediators is p-cresol.

A water-soluble mediator can dissolve in, e.g., a water pool of an enzyme body and exhibit the function of the mediator.

NADH participates in many enzymatic reactions as a coenzyme. In these reactions, NADH provides electrons and protons, and is oxidized to NAD⁺, which is the oxidant form thereof. On the other hand, it is desirable to reuse NADH by reduction from the oxidant NAD⁺ because NADH is very expensive. NADH regenerating methods are under consideration by using methods such as a chemical method, electrochemical method, photoelectrochemical method, and enzymatic method. Of these methods, the NADH regenerating method using the electrochemical method is a useful method because the addition of a reducing reagent is not required and the cost is low.

When regenerating an oxidized mediator by the electrochemical method in the carbon dioxide fixation device according to the embodiment, a cathode electrode and anode electrode are installed, for example, in contact with the nonaqueous phase. As will be described later, the action (e.g., a reduction reaction) by the cathode electrode can regenerate the mediator.

On the other hand, when electrochemically reducing NAD⁺ to NADH by using an ordinary electrode, NAD⁺ is reduced to NADH (a coenzyme) having enzyme activity, and is also reduced to a dimer NAD₂ having no enzyme activity. In addition, the generation rate of the dimer NAD₂ is higher than that of the reaction of reducing NAD⁺ to NADH. When electrochemically reducing NAD⁺, therefore, the dimer NAD₂ is mainly generated by reduction. The dimer NAD₂ is inactive as an enzyme, i.e., has no function as a coenzyme.

By contrast, NADH functions as a coenzyme, but the generation rate is lower than that of the reaction of generating the dimer NAD₂. Therefore, due to generation of the dimer NAD₂, there is gradual reduction in the amount of NADH which can participate in an enzymatic reaction.

Accordingly, it is desirable to use another mediator in order to decrease the reduction potential (overvoltage) of NAD⁺. That is, a mediator capable of reducing NAD⁺ may be dispersed in a medium or water pool in the nonaqueous phase containing NAD⁺. NAD⁺ can be reduced by this mediator. As will be described in detail later, the oxidized mediator can be reduced by, e.g., the action of a cathode electrode.

When using another mediator together with NAD⁺, the other mediator may be, e.g., hydroquinone, ferricyanide, ferrocene, an organic dye, and a transition metal complex. These mediators may be dispersed in the medium in the nonaqueous phase.

Alternatively, a catalyst layer capable of reducing NAD⁺ at a lower overvoltage may be disposed in the nonaqueous phase. This catalyst layer may be disposed, e.g., on a cathode electrode. The catalyst layer may be disposed onto the electrode by a method such as electrolytic polymerization, direct spraying, coating, or impregnation.

For example, an electrolyte membrane of poly(neutral red) [abbreviation: poly(NR)] may be formed on a porous membrane by electrolytic polymerization. By using this porous membrane having the poly(NR) modification membrane, NAD⁺ can be efficiently reduced to NADH having enzyme activity at a relatively low overvoltage (−600 mV vs. Ag/AgCl).

On the other hand, reduction from NAD⁺ to NADH requires not only electrons (e⁻) but also protons (H⁺). The protons can be supplied from, e.g., an aqueous phase as a proton source. A case in which the aqueous phase is used as a proton source will be described in detail later.

In the carbon dioxide fixation device according to the embodiment, when regenerating the oxidized mediator by an enzymatic method, i.e., by using an enzymatic reaction, it is possible to use, e.g., an enzyme body containing an enzyme which catalyzes a reaction that reduces an oxidant of the mediator. As a practical example, an enzyme body containing GDH can be used when NADH/NAD⁺ is adopted as the mediator.

The method of regenerating the mediator is not limited to the specific method explained above, and it is possible to use, e.g., a chemical method, electrochemical method, photoelectrochemical method, or enzymatic method. It is also possible to adopt one mediator regenerating method or a plurality of mediator regenerating methods. For example, the electrochemical method and enzymatic method may be used together.

When using only a mediator other than the abovementioned coenzymes (e.g., NADH), it is possible to use, e.g., ferrocene or the following hydrophobic mediators (decamethyl ferrocene, 1,2-diferrocenylethylene, or tetramethylp-phenylenediamine).

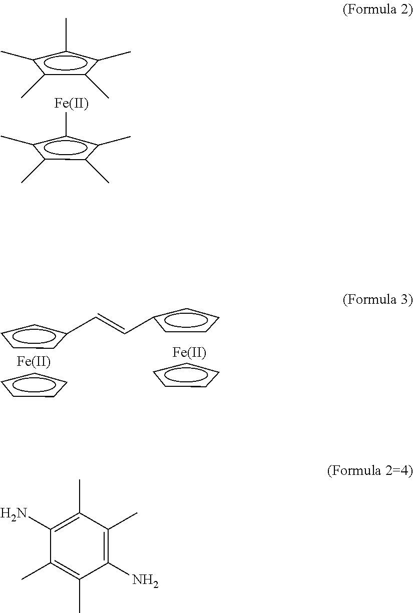

[Electrodes]

As described above, when reducing carbon dioxide or a reduced product (intermediate product) of carbon dioxide in an enzymatic reaction in the carbon dioxide fixation device according to the embodiment, the electrochemical method may be used to regenerate the oxidized mediator. When adopting the electrochemical method in order to reduce and regenerate the mediator, a pair of electrodes called a cathode and anode are installed in the nonaqueous phase. These cathode and anode may be included within, e.g., the medium (ionic liquid) of the nonaqueous phase. Also, a porous electrode may be used as each of the cathode and anode. In such a case, these electrodes can also function as porous membranes.

The cathode electrode has functions of, e.g., regenerating the coenzyme NADH consumed in the enzymatic reaction of carbon dioxide and reducing the oxidized mediator. That is, the cathode electrode functions as a cathode. On the other hand, the anode electrode pairs with the cathode electrode and functions as an anode.

A supporting salt or the like may also be added as an electrolyte to the nonaqueous phase. Examples of the supporting salt include KCl and tetrabutylammonium perchlorate (TBAP).

Note that, some ionic liquids contained in the nonaqueous phase can itself function as an electrolyte. In such a case, it is unnecessary to add an electrolyte for obtaining the functions of the electrodes (cathode and anode).

Also, when using the cathode electrode and anode electrode respectively as a working electrode and a counter electrode, a three-electrode system design may be adopted by disposing a reference electrode or pseudo reference electrode at the nonaqueous phase, in addition to the cathode electrode and anode electrode. Alternatively, it is possible to adopt a design with a four-electrode system by further disposing, in addition to the cathode and anode, two reference electrodes each matched respectively with the cathode and anode, or two pseudo reference electrodes each matched respectively with the cathode and anode. Voltage for reducing the mediator can be controlled more accurately by using the reference electrode or pseudo reference electrode. This makes it possible to efficiently regenerate the mediator. Although the reference electrode and pseudo reference electrode may be installed in an arbitrary position of the nonaqueous phase, they are desirably disposed between the cathode electrode and anode electrode.

The reference electrode and pseudo reference electrode may alternatively be installed in an arbitrary position of the aqueous phase. From the viewpoint of ease of maintenance, the reference electrode and pseudo reference electrode are desirably installed in the aqueous phase. When the reference electrode is installed in the aqueous phase, it is unnecessary to use a reference electrode for an organic solvent.

When a constant electric current is set by appropriately installing the reference electrode and pseudo reference electrode in the fuel generation section as described above, the electric current value and product concentration (e.g., the fuel concentration) become stable, and thus desirable. When a constant voltage is set, it is desirable also because a stable applied potential value can be sustained and thereby a high-purity product (fuel) can be obtained.

When using nonporous electrodes as the cathode electrode and anode electrode, the cathode electrode and anode electrode are desirably installed in positions of the nonaqueous phase and aqueous phase, which do not interfere with the diffusion of a substrate and intermediate product of an enzymatic reaction, fuel, a coenzyme, other mediators, and proton ions. On the other hand, when the cathode electrode and anode electrode can also function as porous membranes, the installation positions of the cathode electrode and anode electrode are not particularly limited.

For example, the cathode electrode may be installed between the nonaqueous phase and aqueous phase. Also, the anode electrode may be installed between the gaseous phase and nonaqueous phase. Furthermore, the nonaqueous phase may be divided into a plurality of regions by installing a plurality of cathodes in the nonaqueous phase. When using the aqueous phase as a proton source for protons to be used to regenerate the mediator, the protons can efficiently be supplied to the cathode electrode by installing the cathode electrode as a porous membrane between the nonaqueous phase and aqueous phase. This can advance the regeneration of the mediator.

The material of the cathode electrode and anode electrode may be selected from electrically conductive materials, and is not particularly limited. For example, it is possible to use commercially available carbon, carbon cloth, carbon fiber, carbon nonwoven, and carbon paper. It is also possible to use a composite electrode material containing thin fragments of graphene or carbon nanotubes. An electrode using such material can also function as a porous membrane. When one surface of an electrode which also functions as a porous membrane is exposed to a gaseous phase such as the atmosphere (air), a gas diffusion layer may also be disposed on the surface exposed to the gaseous phase in order to promote the supply of carbon dioxide to the nonaqueous phase, for example. The gas diffusion layer is a micro porous layer composed mainly of carbon and a water-repellent material.

It is also possible to use electrodes made of metal materials as the cathode electrode and anode electrode. For example, electrodes containing platinum, gold, and silver may be used. On the other hand, the use of a titanium electrode is desirable from the viewpoint of cost.

As the reference electrode and pseudo reference (or quasi-reference) electrode, it is possible to use electrodes such as a platinum electrode, platinum black electrode, palladium electrode, silver electrode, silver/silver chloride (Ag/AgCl) electrode, gold electrode, and carbon electrode.

Practical examples of the material of the cathode electrode and anode electrode are carbon felt, carbon nanofiber nonwoven, and foamed graphene (e.g., graphene foams with continuous 3D networks).

The foamed graphene can be manufactured, for example, by forming a graphene layer on nickel foam by chemical vapor deposition (CVD), and removing the nickel base after that. Since the foamed graphene has many pores, the medium (ionic liquid) of the nonaqueous phase can permeate into the interior of the foamed graphene. The foamed graphene further has merits such as robustness, flexibility, and ease of handling.

The electrodes (including, cathode electrode, anode electrode, reference electrode, quasi-reference electrode, and pseudo reference electrode) may appropriately be processed into shapes such as a plate form, rod form, mesh form, wire form, foam form, and cloth-form in accordance with the installing positions and use of the electrodes.

[Porous Membrane]

A porous membrane may be disposed between the gaseous phase and nonaqueous phase or between the nonaqueous phase and aqueous phase. Also, the nonaqueous phase may be divided into a plurality of nonaqueous phases by installing the porous membrane(s) in the nonaqueous phase.

The porous cathode electrode and porous anode electrode may be used as porous membranes, as described above, but it is also possible to use a porous membrane apart from the cathode electrode and anode electrode. In this case, inorganic or organic materials such as glass, ceramics, and polymers may be used as the material of the porous membrane, in addition to the above-described electrode materials.

Also, an appropriate position of the surface of the porous membrane may be modified with, e.g., a water-repellent membrane, hydrophilic membrane, modification membrane, or metal nanoparticles, as appropriate. Note that when the cathode electrode and anode electrode also function as porous membranes, these electrodes may also be modified with a water-repellent membrane, hydrophilic membrane, modification membrane, or metal nanoparticles.

An example of the modification membrane is modification by electrolytic polymerization of poly(NR).

Furthermore, when installing a porous membrane with respect to the aqueous phase, a permeation-separation membrane, proton exchange membrane (PEM), or protic ionic liquid membrane may further be disposed on that surface of the porous membrane, which faces the aqueous phase.

<Aqueous Phase>

The carbon dioxide fixation device according to the embodiment includes an aqueous phase containing an extraction liquid containing water.

The extraction liquid may include a buffering liquid. The type and concentration of the buffering liquid may be appropriately selected in accordance with the type of reaction system or mediator. A properly adjusted buffering liquid can function as a proton source for regenerating the mediator by reduction.

The extraction liquid is not particularly limited, and an aqueous solution capable of rapidly extracting a reaction product may be used as the extraction liquid. As the extraction liquid, it is desirable to use an aqueous solution capable of continuously supplying protons to the nonaqueous phase.

<Separator>

The carbon dioxide fixation device according to the embodiment may further include a separator. A position where the separator is installed may be changed in accordance with the design of the device. For example, it is possible to install the separator between the nonaqueous phase and aqueous phase, or install the separator adjacent to the electrode which also functions as a porous membrane. However, the installation position of the separator is not limited to these positions.

The separator is desirably selected from materials capable of selectively permeating the substrate, fuel, and mediator. In addition, the separator is desirably selected from insulating materials.

The separator may be selected from one that is independent and one that is a composite. For example, it is possible to use a composite separator where the separator is modified onto a porous membrane and integrated with the porous membrane. Alternatively, an independent separator not integrated with a porous membrane may be used.

When installing the separator between the gaseous phase and nonaqueous phase, e.g., a hydrophobic separator may be used so as to selectively permeate carbon dioxide while not permeating an intermediate product (e.g., formic acid or formaldehyde) or a final product (e.g., fuel such as methanol).

On the other hand, when installing the separator between the nonaqueous phase and aqueous phase, it is desirable to select the separator from a proton exchange membrane and hydrophilic material, in consideration of migration of proton ions from the aqueous phase to the nonaqueous phase and in consideration of diffusion and extraction of fuel from the nonaqueous phase to the aqueous phase.

The separator may be selected from polymer membranes. The polymer membranes may be selected from a homogeneous membrane, composite membrane, and asymmetric membrane. A Nafion® membrane may also be used as the separator. It is particularly desirable to use a permeation-separation membrane which selectively permeates water, reaction products, and ions, and does not permeate enzymes, enzyme bodies, and ionic liquids.

The thickness of the separator may be appropriately selected in accordance with the permeation rate of carbon dioxide in the gaseous phase, and the permeation rates of ions and molecules in the nonaqueous phase and aqueous phase.

A practical configuration example of the carbon dioxide fixation device according to the embodiment will be explained below with reference to the drawings. Note that the same reference numerals denote the same features in all embodiments, and repetitive explanation will be omitted. Note also that each drawing is a schematic view for explanation and promotion of understanding of the embodiment, so shapes, dimensions, ratios, and the like are sometimes different from those of the actual device.

However, the designs of these factors may appropriately be changed by taking account of the above-described explanation and known techniques.

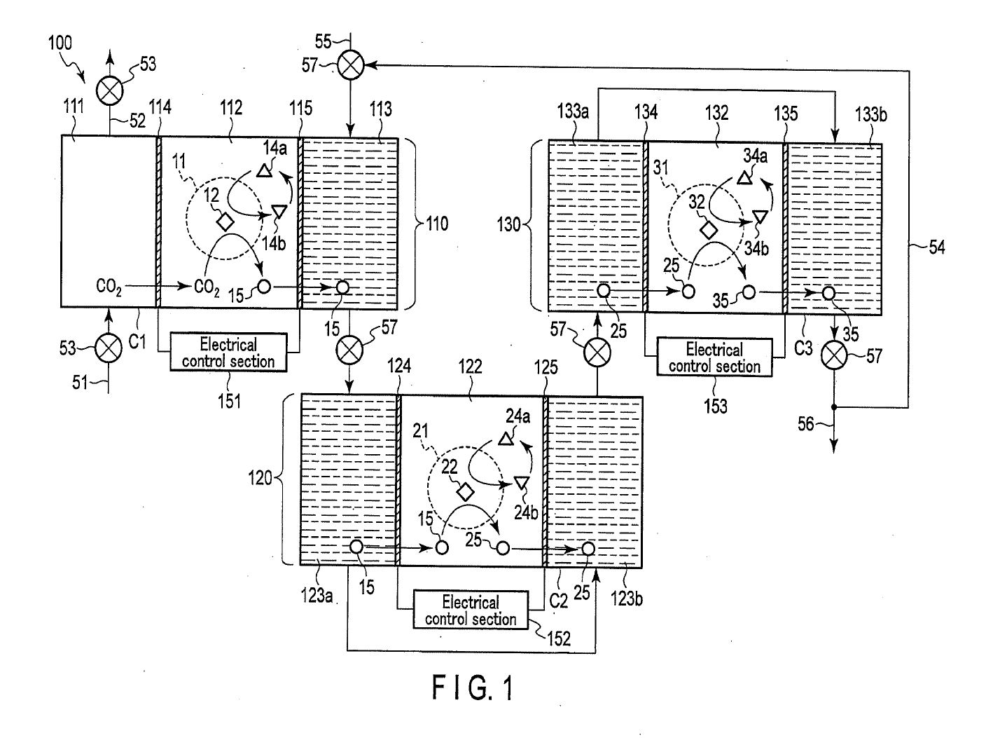

FIG. 1 shows an example of a carbon dioxide fixation device which corresponds to the first mode of the embodiment, and reduces carbon dioxide to a final product through a three-cascade enzymatic reaction.

A carbon dioxide fixation device 100 shown in FIG. 1 includes a first block 110, a second block 120, and a third block 130.

The first block 110 includes a first cell member C1 which accommodates a gaseous phase 111, a nonaqueous phase 112, and an aqueous phase 113, or which has an internal space for accommodating these phases. The second block 120 includes a second cell member C2 which accommodates a first aqueous phase 123a, a nonaqueous phase 122, and a second aqueous phase 123b, or which has an internal space for accommodating these phases. The third block 130 includes a third cell member C3 which accommodates a first aqueous phase 133a, a nonaqueous phase 132, and a second aqueous phase 133b, or which has an internal space for accommodating these phases.

The nonaqueous phase 112 of the first block 110, the nonaqueous phase 122 of the second block 120, and the nonaqueous phase 132 of the third block 130 can respectively be regarded as first, second, and third nonaqueous phases.

Also, the aqueous phase 113 of the first block 110 and the first aqueous phase 123a in the former stage of the second block 120 can together be regarded as a first aqueous phase. The second aqueous phase 123b in the latter stage of the second block 120 and the first aqueous phase 133a in the former stage of the third block 130 can together be regarded as a second aqueous phase. The second aqueous phase 133b in the latter stage of the third block 130 can be regarded as a third aqueous phase.

More specifically, in the first block 110 of the carbon dioxide fixation device 100, the first cell member C1 accommodates the gaseous phase 111 containing carbon dioxide, the nonaqueous phase 112 containing an ionic liquid, an enzyme body 11, and mediators (a reductant 14a and an oxidant 14b), and the aqueous phase 113 containing an extraction liquid for extracting a first reaction product 15 from the nonaqueous phase 112.

In the first block 110, an anode electrode 114 and a cathode electrode 115 configured to reduce the mediator oxidant 14b to the mediator reductant 14a are disposed in contact with the nonaqueous phase 112. The first block 110 further includes a cell voltage control section configured to control voltage (or potential) applied to the cathode electrode 115 and anode electrode 114. In the first block 110, the cathode electrode 115 and anode electrode 114 are electrodes made of a porous material.

The cell voltage control section may be replaced with an electric current control section. The electric current control section is configured to control an electric current which flows between the cathode electrode 115 and anode electrode 114.

The shown electrical control section 151 may be a cell voltage control section configured to control the voltage applied to the cathode electrode 115 and anode electrode 114. Alternatively, the electrical control section 151 may be an electric current control section configured to control the electric current which flows between the cathode electrode 115 and anode electrode 114. The electrical control section 151 may also include a mode in which the control section functions as the cell voltage control section and a mode in which the control section functions as the electric current control section.

The cathode electrode 115 is a cathode, and also functions as a porous membrane being installed between the nonaqueous phase 112 and aqueous phase 113. The anode electrode 114 is an anode, and also functions as a porous membrane being installed between the gaseous phase 111 and nonaqueous phase 112. The cathode electrode 115 and anode electrode 114 partition the internal space of the first cell member C1 into three compartments, i.e., a compartment for the gaseous phase 111, a compartment for the nonaqueous phase 112, and a compartment for the aqueous phase 113. Note that the cathode electrode 115 shown in FIG. 1 is installed between the nonaqueous phase 112 and aqueous phase 113, but may also be installed in another arbitrary position within the nonaqueous phase 112. For example, the cathode electrode 115 is desirably installed near the interface between the nonaqueous phase 112 and aqueous phase 113, without being in contact with the aqueous phase 113. Likewise, the anode electrode 114 may be installed in an arbitrary position within the nonaqueous phase 112. When installed between the nonaqueous phase 112 and gaseous phase 111, the anode electrode 114 may partially be exposed to the gaseous phase.

Although the first block 110 in FIG. 1 shows the arrangement of a two-electrode system, the arrangement of a three-electrode system or four-electrode system may also be adopted. When adopting the three-electrode system or four-electrode system, a reference electrode or pseudo reference electrode may be installed in an arbitrary position of the nonaqueous phase 112 in addition to the cathode electrode 115 and the anode electrode 114. As described above, the use of the reference electrode or pseudo reference electrode makes it possible to more accurately control the voltage for reducing the oxidant 14b of the mediator and efficiently regenerate the mediator.

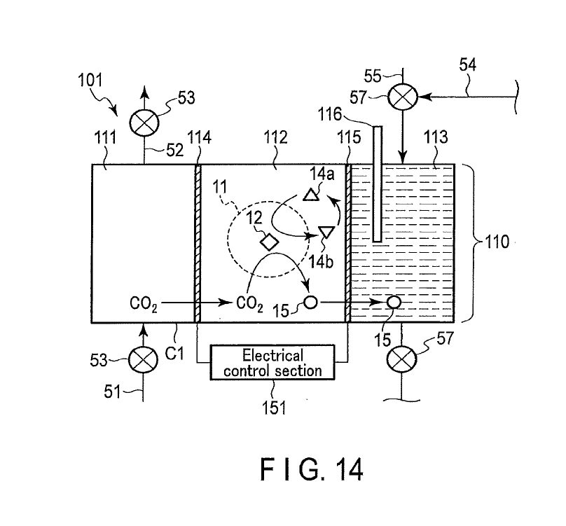

The reference electrode and pseudo reference electrode may also be installed in an arbitrary position of the aqueous phase 113. For example, a reference electrode 116 may be installed in the aqueous phase 113 as in a carbon dioxide fixation device 101 shown in FIG. 14 as an exemplary modification. To simplify the explanation, FIG. 14 abbreviates second and third blocks of the carbon dioxide fixation device 101, and shows only a first block 110. There is no difference between the carbon dioxide fixation device 100 and the carbon dioxide fixation device 101 as a modification except the presence/absence of the reference electrode 116, so details of the rest will be omitted.

The enzyme body 11 shown in FIG. 1 is an enzyme body and contains an enzyme 12. The enzyme 12 catalyzes a reaction where carbon dioxide (CO₂), which is supplied to the nonaqueous phase 112, is reduced to the first reaction product 15.

Carbon dioxide contained in the gaseous phase 111 is supplied to the nonaqueous phase 112 by being selectively absorbed by the ionic liquid contained in the nonaqueous phase 112. The enzymatic action of the enzyme 12 of the enzyme body 11 reduces carbon dioxide to the first reaction product 15. Also, as the action of the enzyme 12 reduces carbon dioxide to the first reaction product 15, the mediator reductant 14a is oxidized to the mediator oxidant 14b. After that, the mediator oxidant 14b is reduced by the cathode electrode 115, and regenerated as the reductant 14a.

Then, the extraction liquid in the aqueous phase 113 extracts the generated first reaction product 15. After that, the first reaction product 15 is introduced into the second block 120. Alternatively, if the first reaction product 15, which is an intermediate product, is itself a useful substance, the first reaction product 15 may alternatively be recovered via the aqueous phase 113 and made use of. Note that when recovering the first reaction product 15, a takeout port (not shown) for taking out the recovered product may be disposed on the cell member C1 at a portion adjacent to the aqueous phase 113.

The flow rate of the gaseous phase may be controlled by air current control valves 53 disposed at a gas supply port (CO₂ inlet) 51 and a gas exhaust port (CO₂ outlet) 52, or a pump (not shown) installed on a gas supply path connected to the gas supply port 51.

It is also possible to control the pressure of the gaseous phase 111 by controlling the air current control valves 53 or the pump installed on the gas supply path. The supply of carbon dioxide to the nonaqueous phase 112 can be accelerated by raising the pressure of the gaseous phase 111.

In the carbon dioxide fixation device 100 shown in FIG. 1, the gaseous phase 111 is accommodated in the first cell member C1. However, it is also possible to adopt a form in which the first cell member C1 does not accommodate the gaseous phase 111. For example, in the carbon dioxide fixation device 100, the nonaqueous phase 112 may alternatively be in contact with the atmosphere, directly, or with the porous anode electrode 114 or another porous membrane being interposed therebetween.

The flow rate of the extraction liquid in the aqueous phase 113 may be controlled by liquid flow control valves 57 installed at an extraction liquid inlet 55 and extraction liquid outlet 56 or a pump (not shown) installed on an external flow path 54 connected to the extraction liquid inlet 55.

In the second block 120 of the carbon dioxide fixation device 100, the second cell member C2 accommodates the first aqueous phase 123a to which is introduced the aqueous phase 113 having extracted the first reaction product 15, the nonaqueous phase 122 containing an ionic liquid, an enzyme body 21, and mediators (a reductant 24a and an oxidant 24b), and the second aqueous phase 123b containing an extraction liquid for extracting a second reaction product 25 from the nonaqueous phase 122. Note that the ionic liquid contained in the nonaqueous phase 122 of the second block 120 may be either the same as or different from the ionic liquid contained in the nonaqueous phase 112 of the first block 110. Also, the mediators (24a and 24b) contained in the nonaqueous phase 122 of the second block 120 may be either the same as or different from the mediators (14a and 14b) contained in the nonaqueous phase 112 of the first block 110.

In the second block 120, an anode electrode 124 and a cathode electrode 125 configured to reduce the mediator oxidant 24b to the mediator reductant 24a are disposed in contact with the nonaqueous phase 122. The second block 120 further includes a cell voltage control section configured to control voltage (or potential) applied to the cathode electrode 125 and anode electrode 124.

The cell voltage control section may be replaced with an electric current control section. The electric current control section is configured to control an electric current which flows between the cathode electrode 125 and anode electrode 124

The shown electrical control section 152 may be a cell voltage control section configured to control the voltage applied to the cathode electrode 125 and anode electrode 124. Alternatively, the electrical control section 152 may be an electric current control section configured to control the electric current which flows between the cathode electrode 125 and anode electrode 124. The electrical control section 152 may also include a mode in which the control section functions as the cell voltage control section and a mode in which the control section functions as the electric current control section.

Note that in the case of a two-electrode system as shown in FIG. 1, the cell voltage control section (electrical control section 152) controls the voltage applied to the electrodes. On the other hand, the cell voltage control section controls the potential applied to the electrodes in the case of, e.g., a three-electrode system. In the second block 120, the cathode electrode 125 and anode electrode 124 are electrodes made of a porous material.

The cathode electrode 125 is a cathode, and also functions as a porous membrane being installed between the nonaqueous phase 122 and second aqueous phase 123b. The anode electrode 124 is an anode, and also functions as a porous membrane being installed between the first aqueous phase 123a and nonaqueous phase 122. Furthermore, the cathode electrode 125 and anode electrode 124 function as support substrates for the nonaqueous phase 122. The cathode electrode 125 and anode electrode 124 partition the internal space of the second cell member C2 into three compartments, i.e., a compartment for the first aqueous phase 123a, a compartment for the nonaqueous phase 122, and a compartment for the second aqueous phase 123b. Note that the cathode electrode 125 and anode electrode 124 shown in FIG. 1 are respectively installed between the first aqueous phase 123a and nonaqueous phase 122, and between the nonaqueous phase 122 and second aqueous phase 123b; however, the cathode electrode 125 and anode electrode 124 may alternatively be installed in other arbitrary positions within the nonaqueous phase 122. For example, the cathode electrode 125 is desirably installed near the interface between the nonaqueous phase 122 and second aqueous phase 123b, without being in contact with the aqueous phase. Likewise, the anode electrode 124 may be installed in an arbitrary position in the nonaqueous phase 122. For example, the anode electrode 124 is desirably installed near the interface between the nonaqueous phase 122 and first aqueous phase 123a, without being in contact with the aqueous phase.

When the extraction liquid containing the first reaction product 15 is introduced from the aqueous phase 113 of the first block 110 into the first aqueous phase 123a of the second block 120, the first reaction product 15 is supplied to the nonaqueous phase 122 by being selectively extracted by the ionic liquid and enzyme body 21 contained in the nonaqueous phase 122. The enzymatic action of the enzyme 22 of the enzyme body 21 reduces the first reaction product 15 to the second reaction product 25. Also, as the action of the enzyme 22 reduces the first reaction product 15 to the second reaction product 25, the mediator reductant 24a is oxidized to the mediator oxidant 24b. After that, the mediator oxidant 24b is reduced by the cathode electrode 125, and regenerated as the reductant 24a.

Then, the extraction liquid in the second aqueous phase 123b extracts the generated second reaction product 25. After that, the second reaction product 25 is introduced into the third block 130. Alternatively, for example, if the second reaction product 25, which is an intermediate product, is itself a useful substance, the second reaction product 25 may also be recovered by way of the second aqueous phase 123b and made use of. Note that, when recovering the second reaction product 25, a takeout port (not shown) for taking out the recovered product may be disposed on the cell member C2 at a portion adjacent to the second aqueous phase 123b.

As in the first block 110, the flow rates of the extraction liquids in the first and second aqueous phases 123a and 123b of the second block 120 may be controlled by the liquid flow control valves 57 or the pump (not shown) installed on the external flow path 54 connected to the extraction liquid inlet 55.

In the third block 130 of the carbon dioxide fixation device 100, the third cell member C3 accommodates the first aqueous phase 133a to which the second aqueous phase 123b of the second block 120 having extracted the second reaction product 25 is introduced, the nonaqueous phase 132 containing an ionic liquid, an enzyme body 31, and mediators (a reductant 34a and an oxidant 34b), and the second aqueous phase 133b containing an extraction liquid for extracting a third reaction product 35 from the nonaqueous phase 132. Note that the ionic liquid contained in the nonaqueous phase 132 of the third block 130 may be either the same as or different from the ionic liquid contained in the nonaqueous phase 112 of the first block 110 and the ionic liquid contained in the nonaqueous phase 122 of the second block 120. Also, the mediators (34a and 34b) contained in the nonaqueous phase 132 of the third block 130 may be either the same as or different from the mediators (14a and 14b) contained in the nonaqueous phase 112 of the first block 110 and the mediators (24a and 24b) contained in the nonaqueous phase 122 of the second block 120.

In the third block 130, a cathode electrode 135 for reducing the mediator oxidant 34b to the mediator reductant 34a and an anode electrode 134 are disposed in contact with the nonaqueous phase 132. The third block 130 further includes a cell voltage control section configured to control voltage (or potential) applied to the cathode electrode 135 and anode electrode 134. In the third block 130, the cathode electrode 135 and anode electrode 134 are electrodes made of a porous material.

The cell voltage control section may be replaced with an electric current control section. The electric current control section is configured to control an electric current which flows between the cathode electrode 135 and anode electrode 134.

The shown electrical control section 153 may be a cell voltage control section configured to control the voltage applied to the cathode electrode 135 and anode electrode 134. Alternatively, the electrical control section 153 may be an electric current control section configured to control the electric current which flows between the cathode electrode 135 and anode electrode 134. The electrical control section 153 may also include a mode in which the control section functions as the cell voltage control section and a mode in which the control section functions as the electric current control section.

The cathode electrode 135 is a cathode, and also functions as a porous membrane being installed between the nonaqueous phase 132 and second aqueous phase 133b. The anode electrode 134 is an anode, and also functions as a porous membrane being installed between the first aqueous phase 133a and nonaqueous phase 132. Furthermore, the cathode electrode 135 and anode electrode 134 function as support substrates for the nonaqueous phase 132. The cathode electrode 135 and anode electrode 134 partition the internal space of the third cell member C3 into three compartments, i.e., a compartment for the first aqueous phase 133a, a compartment for the nonaqueous phase 132, and a compartment for the second aqueous phase 133b. Note that the cathode electrode 135 and anode electrode 134 shown in FIG. 1 are respectively installed between the first aqueous phase 133a and nonaqueous phase 132, and between the nonaqueous phase 132 and second aqueous phase 133b; however, the cathode electrode 135 and anode electrode 134 may also be installed in other arbitrary positions within the nonaqueous phase 132. Note also that the cathode electrode 135 and anode electrode 134 of the third block may be installed in the nonaqueous phase 132 by the same arrangement as that for the cathode electrode 125 and anode electrode 124 of the second block.

When the extraction liquid containing the second reaction product 25 is introduced from the second aqueous phase 123b of the second block 120 into the first aqueous phase 133a of the third block 130, the second reaction product 25 is supplied to the nonaqueous phase 132 by being selectively extracted by the ionic liquid contained in the nonaqueous phase 132. The enzymatic action of the enzyme 32 of the enzyme body 31 reduces the second reaction product 25 to the third reaction product 35. Also, as the action of the enzyme 32 reduces the second reaction product 25 to the third reaction product 35, the mediator reductant 34a is oxidized to the mediator oxidant 34b. After that, the mediator oxidant 34b is reduced by the cathode electrode 135, and regenerated as the reductant 34a.

Then, the extraction liquid in the second aqueous phase 133b extracts the generated third reaction product 35. After that, the third reaction product 35 is recovered as a final product. For example, the third reaction product 35 as a final product may be recovered from an extraction liquid outlet 56.

As shown in FIG. 1, the extraction liquid taken out from the second aqueous phase 133b of the third block 130 may also be reintroduced into the aqueous phase 113 of the first block 110 through an external flow path 54. In this case, the third reaction product 35 may also be condensed and recovered by installing on the external flow path 54, e.g., an apparatus (not shown) which condenses the third reaction product 35. It is also possible to appropriately adjust the pH of the extraction liquid by connecting a pH adjusting line (not shown) to the external flow path 54 through which the extraction liquid flows.

As in the first and second blocks 110 and 120, the flow rates of the extraction liquids in the first and second aqueous phases 133a and 133b of the third block 130 may be controlled by the liquid flow control valves 57 or the pump (not shown) installed on the external flow path 54 connected to the extraction liquid inlet 55.

In the carbon dioxide fixation device 100 shown in FIG. 1, each of the first, second, and third blocks 110, 120, and 130 includes the cell voltage control section (electrical control section 151, 152, or 153) configured to control the voltage (or potential) applied to the electrodes. However, it is also possible to use one cell voltage control section which comprehensively controls the voltages (or potentials) for all of the first, second, and third blocks 110, 120, and 130.

Also in the case where the electrical control section functions as an electric current control section, there may be used one electric current control section (electrical control section) which comprehensively controls the electric current for all of the first, second, and third blocks 110, 120, and 130.

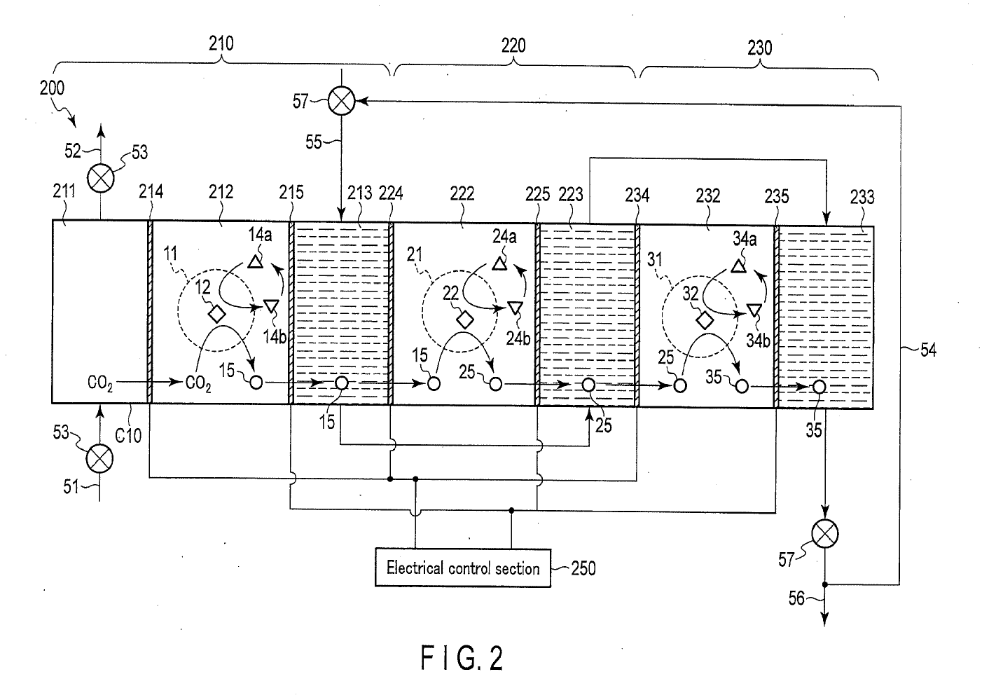

FIG. 2 is a schematic view of a carbon dioxide fixation device of another example of the first mode according to the embodiment.

Like the carbon dioxide fixation device 100 shown in FIG. 1, a carbon dioxide fixation device 200 shown in FIG. 2 includes a first block 210, a second block 220, and a third block 230. Like FIG. 1, the first block 210 of the carbon dioxide fixation device 200 includes a gaseous phase 211, a nonaqueous phase 212, and an aqueous phase 213. On the other hand, the second block 220 includes a nonaqueous phase 222 and an aqueous phase 223, and the third block 230 includes a nonaqueous phase 232 and an aqueous phase 233.

As shown in FIG. 2, the first, second, and third blocks 210, 220, and 230 are accommodated in a single cell member C10.

The nonaqueous phase 212 of the first block 210, the nonaqueous phase 222 of the second block 220, and the nonaqueous phase 232 of the third block 230 can be regarded as a first nonaqueous phase, second nonaqueous phase, and third nonaqueous phase, respectively. Also, the aqueous phase 213 of the first block 210, the aqueous phase 223 of the second block 220, and the aqueous phase 233 of the third block 230 can be regarded as a first aqueous phase, second aqueous phase, and third aqueous phase, respectively.

In the carbon dioxide fixation device 200, the aqueous phase 213 of the first block 210 is in contact with the nonaqueous phase 222 of the second block 220 as well, as shown in FIG. 2. After an extraction liquid contained in the aqueous phase 213 of the first block 210 extracts a first reaction product 15 generated in the nonaqueous phase 212 of the first block, the first reaction product 15 is directly supplied to the nonaqueous phase 222 of the second block 220, and functions as a substrate of an enzymatic reaction in the nonaqueous phase 222. Similarly, the aqueous phase 223 of the second block 220 is in contact with the nonaqueous phase 232 of the third block 230 as well. After an extraction liquid contained in the aqueous phase 223 of the second block 220 extracts a second reaction product 25 generated in the nonaqueous phase 222 of the second block, the second reaction product 25 is directly supplied to the nonaqueous phase 232 of the third block 230, and functions as a substrate of an enzymatic reaction in the nonaqueous phase 232.

The arrangement of the carbon dioxide fixation device 200 shown in FIG. 2 is designed to be more compact, and can manufacture a third reaction product 35 as a final product of a cascade enzymatic reaction with high productivity.

In the arrangement shown in FIG. 2, a flow rate control method for the gaseous phase 211, a recovery method for the final product (third reaction product) from the aqueous phase 233 of the third block 230, and pH adjusting method are the same as those described with reference to FIG. 1. A liquid flow control valve 57 installed at an extraction liquid inlet 55 controls the flow rate of the extraction liquid.

Furthermore, an anode electrode 214 and a cathode electrode 215 for regenerating a mediator consumed by the enzymatic reaction are installed in contact with the nonaqueous phase 212. Likewise, a cathode electrode 225 and an anode electrode 224 are installed in contact with the nonaqueous phase 222. In addition, a cathode electrode 235 and an anode electrode 234 are installed in contact with the nonaqueous phase 232. The cathode electrodes (215, 225, and 235) as cathodes and the anode electrodes (224 and 234) as anodes, except the anode electrode 214 in the nonaqueous phase 212, are so installed as to partition nonaqueous phases and aqueous phases in their respective nonaqueous phases (212, 222, and 232), as shown in FIG. 2. The anode electrode 214 as an anode is installed between the nonaqueous phase 212 and gaseous phase 211.

A manner of installing the cathode electrodes (215, 225, and 235) in the nonaqueous phases (212, 222, and 232) shown in FIG. 2 is the same as that shown in FIG. 1. In the carbon dioxide fixation device 200 shown in FIG. 2, one cell voltage control section (electrical control section 250) may comprehensively control voltage (or potential) applied to the cathode electrode and anode electrode in each of the blocks (first, second, and third blocks 210, 220, and 230), as shown in FIG. 2. It is alternatively possible to control the voltage (or potential) separately by a cell voltage control section installed for each block, as shown in FIG. 1.

As a matter of course, when the electrical control section 250 functions as an electric current control section, one electric current control section (electrical control section) may comprehensively control an electric current which flows between the cathode electrode and anode electrode for all blocks. Alternatively, an electric current control section (electrical control section) may be installed for each block, to separately control electric current in each of the blocks.

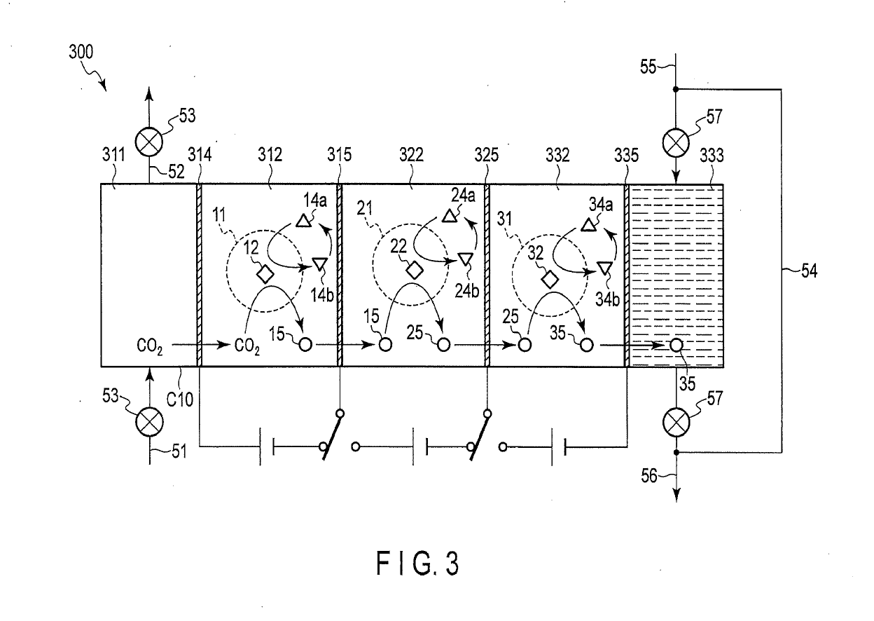

FIG. 3 is a schematic view of a carbon dioxide fixation device as an example of the second mode according to the embodiment.

As shown in FIG. 3, a carbon dioxide fixation device 300 as an example of the second mode according to the embodiment includes a single cell member C10 which accommodates a gaseous phase 311, a first nonaqueous phase 312, a second nonaqueous phase 322, a third nonaqueous phase 332, and an aqueous phase 333. An electrode 315 which also functions as a porous membrane partitions the first nonaqueous phase 312 and second nonaqueous phase 322. Also, an electrode 325 which also functions as a porous membrane partitions the second nonaqueous phase 322 and third nonaqueous phase 332.

To efficiently advance the generation of an intermediate product and final product in each cascade of a cascade enzymatic reaction, it is possible to appropriately adjust the volume (amount) of each nonaqueous phase and the concentration of the enzyme body(s) (11, 21, or 31) dispersed in the nonaqueous phase in accordance with the activity and catalytic efficiency of enzyme(s) (12, 22, or 32) dispersed in the nonaqueous phase.

Like the carbon dioxide fixation devices (100 and 200) as examples of the first mode shown in FIGS. 1 and 2, in the carbon dioxide fixation device 300 shown in FIG. 3, mediators consumed in enzymatic reactions in the nonaqueous phases (312, 322, and 332) are regenerated by being reduced by the electrodes (315, 325, and 335) installed among the nonaqueous phases.

In the carbon dioxide fixation device 300, when the electrode 315 functions as a cathode, the electrode 314 functions as an anode. The electrode 315 functions as an anode for the electrode 325 which functions as a cathode. Also, the electrode 325 functions as an anode for the electrode 335 which functions as a cathode.

The potential (voltage) may sequentially be applied to the electrode pairs from left to right. In other words, the potential (voltage) is first applied for a predetermined time between the electrode 315 which functions as a cathode and the electrode 314 which functions as an anode, thereby reducing an oxidized mediator. After that, the potential is applied between the electrode 325 as a cathode and the electrode 315 as an anode for a predetermined time. The potential is then applied between the electrode 335 as a cathode and the electrode 325 as an anode for a predetermined time.

Thus, the potential (or voltage) may intermittently and sequentially be applied through a plurality of rounds to one or more pairs among the electrodes (314, 315, 325, and 335). In this case, an electrode which acts as a cathode electrode when applying the potential (or voltage) in one round may act as an anode electrode when applying the potential (voltage) in another round. In the above example, the potential (voltage) is sequentially applied from the left to the right. However, the order of applying potential (voltage) to the electrode pairs is not limited to this order. For example, the applying order may be controlled in accordance with the degree of progress of the enzymatic reaction in each nonaqueous phase.

As described above, when applying the potential (voltage) to each electrode pair, the applying of potential is intermittently and sequentially repeated by setting a time difference. This makes it possible to regenerate the oxidant of the mediator in each nonaqueous phase by reducing the oxidant to a reductant, thereby maintaining the enzymatic reaction.

As shown in FIG. 3, the applying of potential (voltage) to each electrode pair may be switched by using a switching mechanism or the like. Each electrode of each electrode pair may alternatively be electrically connected to a cell voltage control section (electrical control section) via independent electrical lines (not shown). In this case, the potential (voltage) may be applied to each electrode pair by setting a predetermined time difference by automatic control using the cell voltage control section.

When using an electric current control section, an electrical current may be supplied or shut off between the electrode pairs by switching the circuits by turning a switch on or off. Each electrode of each electrode pair may alternatively be electrically connected to the electric current control section via independent electrical lines. In this case, an electric current may be flown between the electrode pairs by setting a predetermined time difference by automatic control using the electric current control section.

Also, use of one electrode pair, i.e., two electrodes would be sufficient. As a practical example, two of the four electrodes (314, 315, 325, and 335) shown in FIG. 3 may be omitted from the carbon dioxide fixation device 300. For example, the electrodes 315 and 325 are omitted, and only the electrodes 314 and 335 are installed. Alternatively, the electrodes 314 and 325 are omitted, and only the electrodes 315 and 335 are installed. There may be used one electrode pair, i.e., two or more electrodes, and the number of electrodes to be installed in the device is not particularly limited.

When omitting an electrode installed at the interface of nonaqueous phases, different nonaqueous phases come in direct contact with each other. Furthermore, when omitting an electrode which also functions as a porous membrane, a separator or the like may also be disposed between different nonaqueous phases. Alternatively, a porous membrane such as a separator may also be omitted in order to promote the diffusion of molecules and ions of reaction products and mediators.

When a gas containing carbon dioxide is introduced into the gaseous phase 311, carbon dioxide is selectively supplied to the first nonaqueous phase 312 by the action of an ionic liquid contained in the first nonaqueous phase 312. By the enzymatic action of the enzyme 12 of the enzyme body 11, carbon dioxide is reduced to a first reaction product 15.

The generated first reaction product 15 is further diffused into the second nonaqueous phase 322 and reduced to a second reaction product 25 by the enzyme 22 of the enzyme body 21. The generated second reaction product 25 is further diffused into the third nonaqueous phase 332 and reduced to a third reaction product 35 by the enzyme 32 of the enzyme body 31.

Then, an extraction liquid in the aqueous phase 333 extracts the generated third reaction product 35. After that, the third reaction product 35 is recovered as a final product. For example, the third reaction product 35 as a final product may be recovered from an extraction liquid outlet 56.

After the extraction liquid containing the third reaction product is drawn out from the aqueous phase 333, the extraction liquid may also be passed through an external flow path 54 and reintroduced into the aqueous phase 333 from an inlet, as shown in FIG. 3. In this case, the third reaction product 35 may be condensed and recovered by installing an apparatus (not shown) for condensing the third reaction product 35 on the external flow path 54. It is also possible to appropriately adjust the pH of the extraction liquid by connecting a pH adjusting line (not shown) to the external flow path 54 through which the extraction liquid flows.

In the carbon dioxide fixation device 300, as in the examples (FIGS. 1 and 2) of the first mode of the embodiment, the flow rate of the gaseous phase may be controlled by air current control valves 53 installed at a gas supply port (CO₂ inlet) 51 and a gas exhaust port (CO₂ outlet) 52, or a pump (not shown) installed on a gas supply path connected to the gas supply port 51. In addition, like the carbon dioxide fixation device 100 shown in FIG. 1, it is also possible to not accommodate the gaseous phase 311 in the cell member C10, and for example, expose the first nonaqueous phase 312 to the atmosphere.

As in the examples of the first mode, the flow rate of the extraction liquid in the aqueous phase 333 may be controlled by liquid flow control valves 57 installed at an extraction liquid inlet 55 and extraction liquid outlet 56, or a pump (not shown) installed on an external flow path 54 connected to the extraction liquid inlet 55.

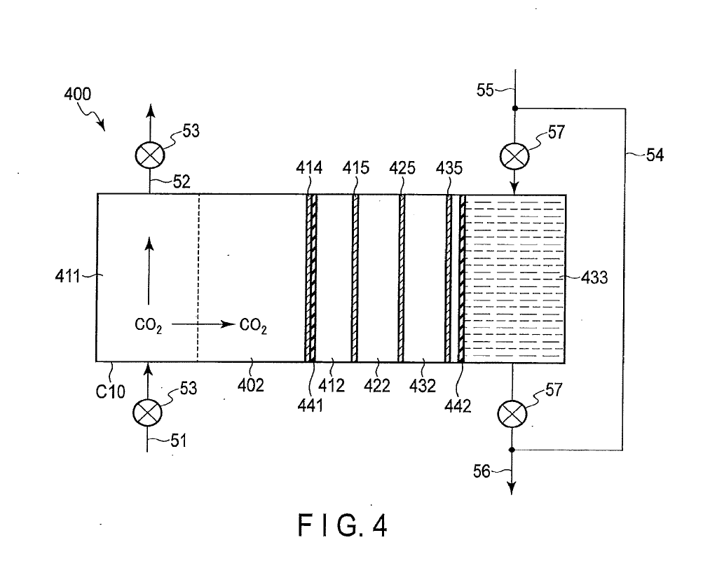

FIG. 4 is a schematic view of a carbon dioxide fixation device of another example of the second mode according to the embodiment.

The basic configuration of a carbon dioxide fixation device 400 shown in FIG. 4 is the same as that of the carbon dioxide fixation device 300 shown in FIG. 3. However, FIG. 4 further includes a fourth nonaqueous phase 402 containing no enzyme body, in addition to a first nonaqueous phase 412, a second nonaqueous phase 422, and a third nonaqueous phase 432, each of which contains enzyme body(s) and mediator(s). Note that although not shown in FIG. 4 for the sake of descriptive simplicity, each of the first, second, and third nonaqueous phases 412, 422, and 432 contains one or more kinds of enzyme bodies and one or more kinds of mediators. Note also that although not shown in FIG. 4 for the sake of descriptive simplicity, an electrical control section (a cell voltage control section and/or an electric current control section) is electrically connected to each of electrodes 415, 425, 435, and 414 shown in FIG. 4.

The fourth nonaqueous phase 402 disposed at former stage relative to the first nonaqueous phase 412 may contain an ionic liquid having functions of absorbing and condensing carbon dioxide from a gaseous phase 411. Carbon dioxide can stably be supplied to the first nonaqueous phase 412 containing an enzyme body by providing such a fourth nonaqueous phase 402.

It is also possible to have the fourth nonaqueous phase 402 be a gelled nonaqueous phase by gelling the ionic liquid contained in the fourth nonaqueous phase 402, and support the gelled nonaqueous phase on the surface of, e.g., the electrode 414. This makes it possible to increase the contact area between the gaseous phase 411 and fourth nonaqueous phase 402, and improve the carbon dioxide absorption efficiency of the fourth nonaqueous phase 402. On the other hand, when using the fourth nonaqueous phase 402 as a liquid nonaqueous phase without gelling the ionic liquid contained in the fourth nonaqueous phase 402, the gaseous phase 411 and fourth nonaqueous phase 402 may be partitioned by disposing, e.g., a porous membrane between the gaseous phase 411 and fourth nonaqueous phase 402. It is also possible to adopt a form in which the gaseous phase 411 is not accommodated in a cell member C10.

Furthermore, as shown in FIG. 4, a separator 441 may also be disposed adjacent to the electrode 414 installed between the first nonaqueous phase 412 and fourth nonaqueous phase 402. As the separator 441, it is desirable to use a material capable of selectively permeating carbon dioxide.

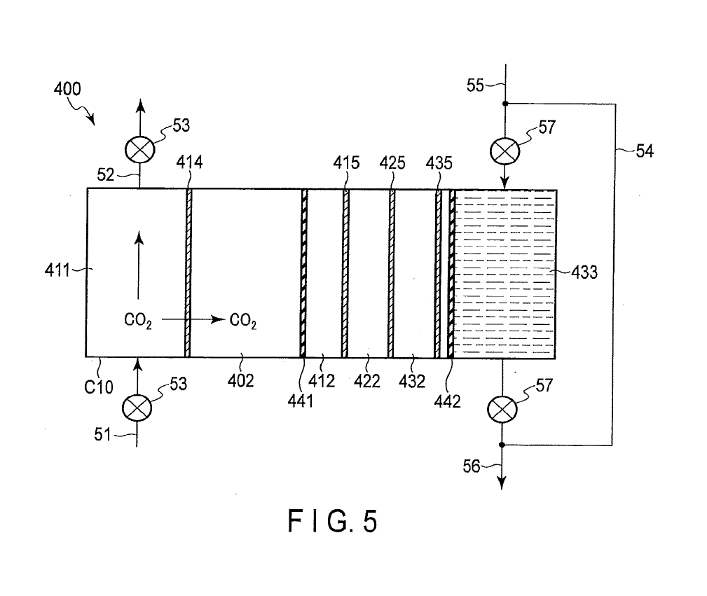

The electrode 414 and separator 441 need not be adjacent to each other. For example, as shown in FIG. 5, the separator 441 may be installed between the first nonaqueous phase 412 and fourth nonaqueous phase 402, and the electrode 414 may be installed between the gaseous phase 411 and fourth nonaqueous phase 402.

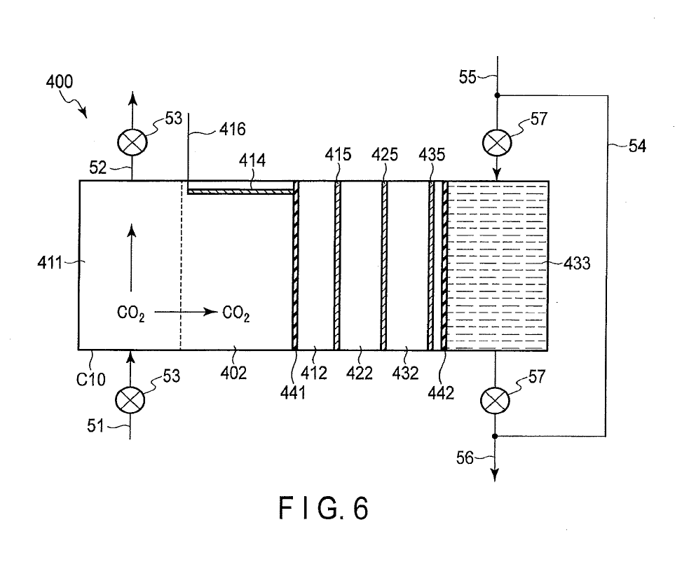

Also, the electrode 414 may be disposed in an arbitrary position in the fourth nonaqueous phase 402. For example, as shown in FIG. 6, the electrode 414 may be installed within the fourth nonaqueous phase 402 along an exterior member of the cell member C10. Note that depending on the form of installing the electrode 414, e.g., a conducting wire 416 as shown in FIG. 6 may be used to secure electrical connection to the electrode 414. The electrode 414 may be disposed in an arbitrary position in the first nonaqueous phase 412.

As shown in FIGS. 4, 5, and 6, a separator 442 may also be disposed between the third nonaqueous phase 432 and an aqueous phase 433. As the separator 442, it is desirable to use a material capable of selectively permeating a final product (third reaction product) of a cascade reduction reaction of carbon dioxide. When the aqueous phase 433 contains a proton source for supplying protons to be used to regenerate the mediator, the separator 442 is desirably capable of permeating protons as well.

The arrangements of the plurality of nonaqueous phases and the electrode layout in the carbon dioxide fixation device 400 shown in FIGS. 4, 5, and 6 are similar to those of the carbon dioxide fixation device 300 shown in FIG. 3. Therefore, the same conditions and procedures as shown in FIG. 3 may be used as a method of applying the potential (voltage) to each cathode and each anode.

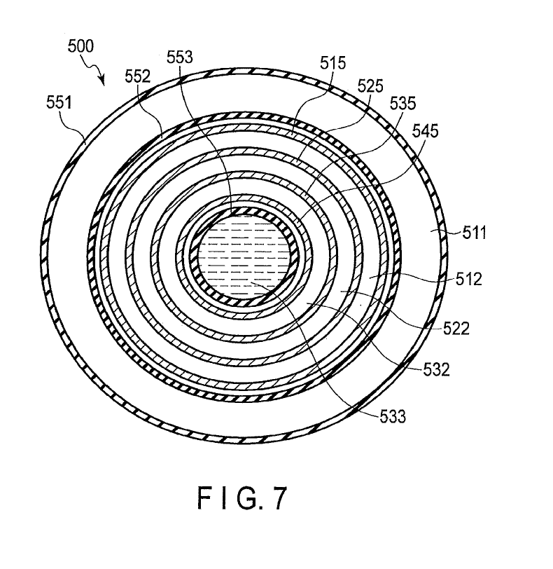

FIG. 7 is a schematic view of a carbon dioxide fixation device of still another example of the second mode according to the embodiment.

Like the carbon dioxide fixation device 300 shown in FIG. 3, a carbon dioxide fixation device 500 shown in FIG. 7 includes a gaseous phase 511, a first nonaqueous phase 512, a second nonaqueous phase 522, a third nonaqueous phase 532, and an aqueous phase 533.

As shown in FIG. 7, the gaseous phase 511 flows through a gas flow path configured from an outer tube 551 and an intermediate tube 552. Each of the first, second, and third nonaqueous phases 512, 522, and 532 has a tubular shape. Also, these tubular nonaqueous phases are so accommodated as to be arranged in order along a direction perpendicular to the central axis (not shown) of the tubes, i.e., along the radial direction of the tube structure. More specifically, the first nonaqueous phase 512 is installed outermost among the tube structure. The third nonaqueous phase 532 is installed innermost among the tube structure. The second nonaqueous phase 522 is installed between the first and third nonaqueous phases 512 and 532. The aqueous phase 533 flows through an inner tube 553 as a liquid flow path.

The outer tube 551 may itself be made of a material having permeability to a gas containing carbon dioxide, and may also be made of a material having no permeability to gas. When the outer tube 551 has no permeability to gas, it is desirable to dispose a gas inlet for introducing gas containing carbon dioxide in between the outer tube 551 and intermediate tube 552.

As the intermediate tube 552 and inner tube 553, it is possible to use a tube obtained using the material of a porous membrane, such as a separator.

As the intermediate tube 552, the use of a material having permeability to carbon dioxide is desirable. As the inner tube 553, the use of a material having permeability to a reduced product of carbon dioxide is desirable. For example, a permeation-separation membrane may be used.

Referring to FIG. 7, the first nonaqueous phase 512 and gaseous phase 511 are in contact with the intermediate tube 552 being interposed therebetween. However, the intermediate tube 552 may be omitted. Similarly, in FIG. 7, the third nonaqueous phase 532 and aqueous phase 533 are in contact with the inner tube 553 being interposed therebetween. However, the inner tube 553 may be omitted.

Note that for the sake of descriptive simplicity as in FIG. 4, FIG. 7 does not show the one or more kinds of enzymes, or one or more kinds of enzyme bodies, and one or more kinds of mediators, contained in each of the first, second, and third nonaqueous phases 512, 522, and 532.

One or more pairs of electrodes (515, 525, 535, and 545) may be installed in the carbon dioxide fixation device 500 shown in FIG. 7. When installing one or more pairs of electrodes, electrically insulating separators (not shown) may be installed between the electrodes in the same manner as shown in FIG. 3, so that the electrodes do not become electrically short-circuited with each other. Note that no separators need be disposed if the nonaqueous phases are thick and there is no possibility of the occurrence of any electrical short-circuits by contact between the electrodes.

When one or more pairs of electrodes are installed, a method (not shown) of applying a potential (voltage) to a cathode electrode may be the same as the applying method shown in FIG. 3.

Although not shown for the sake of descriptive simplicity, an electrical control section (a cell voltage control section and/or electric current control section) is electrically connected to each electrode shown in FIG. 7.

In the gas flow path configured from the outer tube 551 and intermediate tube 552 of the carbon dioxide fixation device 500, the gaseous phase 511 (a gas containing carbon dioxide) flows in a direction along the central axis (not shown) of the outer tube 551 and intermediate tube 552. Also, in the inner tube 553 as a liquid flow path, the aqueous phase 533 (an extraction liquid) flows in a direction along the central axis (not shown) of the inner tube 553.

Note that FIG. 7 shows a form in which the flow path on the outer side of the nonaqueous phase is the gaseous phase, and the flow path on the inner side of the nonaqueous phase is the aqueous phase. However, it is also possible to use the flow path on the inner side of the nonaqueous phase as the gaseous phase, and the flow path on the outer side of the nonaqueous phase as the aqueous phase.

As described above, the carbon dioxide fixation device 500 includes the flow path (the inner tube 553) through which the aqueous phase 533 flows, and the nonaqueous phase is adjacent to the aqueous phase 533 in directions perpendicular to the flow direction of the aqueous phase 533. Also, the carbon dioxide fixation device 500 includes the flow path (the outer tube 551 and intermediate tube 552) through which the gaseous phase 511 flows, and the nonaqueous phase is adjacent to the gaseous phase 511 in directions perpendicular to the flow direction of the gaseous phase 511.

Since the interface between the aqueous phase 533 and nonaqueous phase can be formed along the length of the flow path through which the aqueous phase 533 flows, the contact area between the aqueous phase 533 and nonaqueous phase can be increased. This makes it possible to improve the efficiency at which the final product is supplied to the aqueous phase 533, thereby increasing the recovery rate.

In addition, the interface between the gaseous phase 511 and nonaqueous phase can be formed along the length of the flow path through which the gaseous phase 511 flows, so the contact area between the gaseous phase 511 and nonaqueous phase can be increased. Accordingly, the efficiency at which carbon dioxide is supplied to the nonaqueous phase can be improved.

By using the carbon dioxide fixation device including the aqueous phase 533, the nonaqueous phases (512, 522, and 532), and the gaseous phase 511, which are tubular as shown in FIG. 7, methanol (CH₃OH) can be generated from carbon dioxide (CO₂) by, e.g., a cascade enzymatic reaction indicated by the following formula:

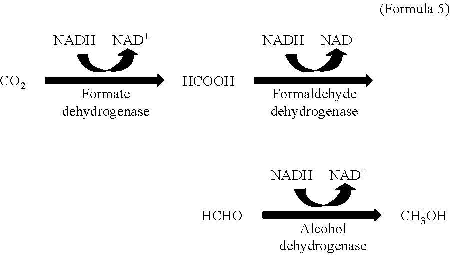

Each of the reaction cascades from the left to the right in the above formula for the three-cascade enzymatic reaction of producing methanol are respectively performed in the first nonaqueous phase 512 installed outermost among the nonaqueous phases shown in FIG. 7, in the second nonaqueous phase 522 installed inside the first nonaqueous phase 512, and in the third nonaqueous phase 532 installed innermost among the nonaqueous phases, sequentially.

By operating electrodes which also function as porous membranes sandwiching the nonaqueous phases as cathodes or working electrodes, NAD⁺ consumed in the enzymatic reaction is regenerated at the same time. Also, in order to efficiently progress the generation of fuel by the cascade enzymatic reaction, it is possible to appropriately adjust the volume (amount) of each nonaqueous phase and the concentration of the enzyme(s) dispersed in the nonaqueous phase in accordance with the activity and catalytic efficiency of the enzyme(s) dispersed in the nonaqueous phase. For example, the method shown in FIG. 3 is applicable as a method of applying the voltage (or potential) to the cathode electrode (or working electrode).

The first reaction product (formic acid; HCOOH), which is generated in the first enzymatic reaction using carbon dioxide as a substrate in the first nonaqueous phase 512, diffuses to the second nonaqueous phase 522 by concentration diffusion, and serves as a substrate of the second enzymatic reaction. Similarly, the second reaction product (formaldehyde; HCHO), which is generated by the second enzymatic reaction using the first reaction product as a substrate in the second nonaqueous phase 522, diffuses to the third nonaqueous phase 532 innermost in the tube by concentration diffusion, and serves as a substrate of the third enzymatic reaction. The third reaction product (methanol; CH₃OH), which is generated by the third enzymatic reaction using the second reaction product as a substrate in the third nonaqueous phase 532, i.e., a final product, is recovered by being extracted by the extraction liquid (aqueous phase 533) flowing through the inner tube 553.

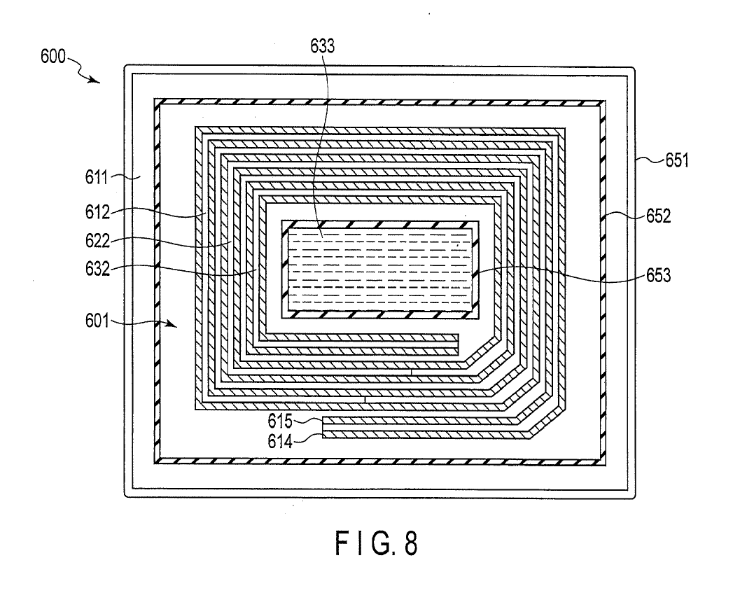

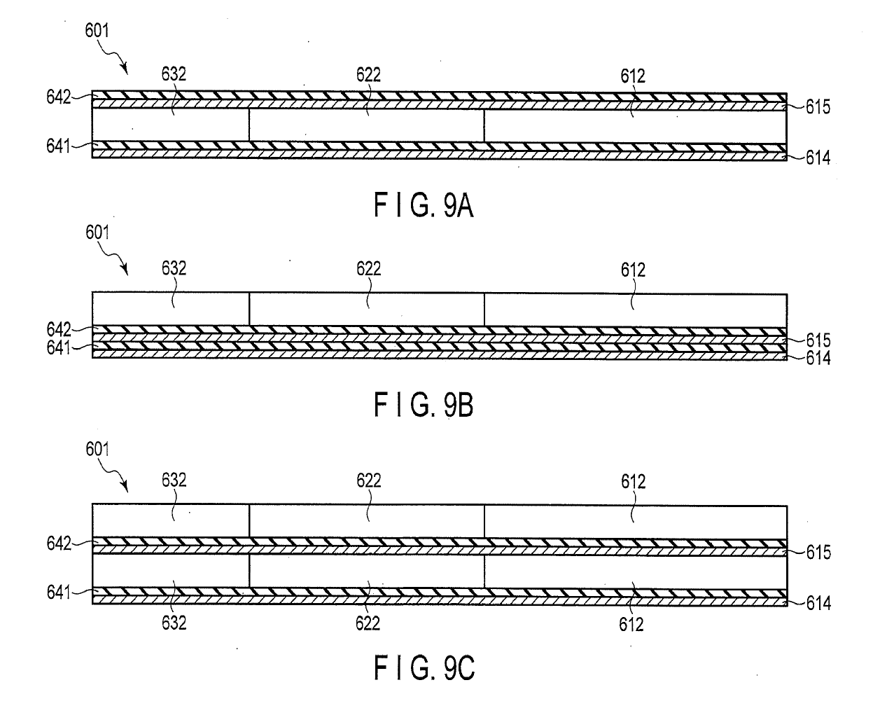

FIG. 8 is a schematic view of a carbon dioxide fixation device of still another example of the second mode according to the embodiment. FIGS. 9A, 9B, and 9C each show an intermediate state during manufacture of an example of a nonaqueous phase which may be included in this device.

A carbon dioxide fixation device 600 shown in FIG. 8 includes a gaseous phase 611, a nonaqueous phase member 601, and an aqueous phase 633.

The gaseous phase 611 flows through a gas flow path defined by an outer tube 651 and an intermediate tube 652. The intermediate tube 652 may also be omitted. In this case, the outer tube 651 and nonaqueous phase member 601 define the gas flow path.

The nonaqueous phase member 601 includes a first nonaqueous phase 612, a second nonaqueous phase 622, a third nonaqueous phase 632, an electrode 614, and an electrode 615. The nonaqueous phase member 601 has a shape in which a plurality of layer sections are wound a plurality of times so as to be arranged in the radial direction, and the first, second, and third nonaqueous phases 612, 622, and 632 are arranged respectively among the plurality of layer sections. Note that the interior of the intermediate tube 652 may be filled with a nonaqueous solvent such as an ionic liquid.

Also, a central portion of the nonaqueous phase member 601, i.e., a space around the winding axis may be used as a flow path. The aqueous phase 633 flows through the flow path formed by the nonaqueous phase member 601 having the wound shape as described above. It is also possible to dispose an inner tube 653 as a flow path of the aqueous phase 633 as shown in FIG. 8.

As shown in FIGS. 9A, 9B, and 9C each showing the intermediate state during which the nonaqueous phase member 601 is being manufactured, more specifically, the state before the nonaqueous phase member 601 is wound, the nonaqueous phase member 601 includes a separator 641 and a separator 642 in addition to the nonaqueous phases and electrodes. The separator 641 is disposed between the electrodes 615 and 614. In the nonaqueous phase member 601 having configurations shown in FIGS. 9A and 9C, the separator 641 is in contact with that surface of the electrode 614, which faces the electrode 615. Note that in an example shown in FIG. 9B, the separator 641 is in contact with both the electrodes 614 and 615. As long as the disposing position of the separator 641 is positioned between the electrodes 615 and 614, the disposing position is not limited to the positions shown in the drawings. For example, the separator 641 may alternatively be in contact with a surface of the electrode 615 (a surface which faces the electrode 614). Note that both the electrodes 615 and 614 are porous electrodes.

The nonaqueous phase member 601 further includes another separator 642 so that the electrodes 615 and 614 would not be in contact with each other after winding. That is, in the wound nonaqueous phase member 601, the members other than the nonaqueous phases, i.e., the electrode 614, separator 641, electrode 615, and separator 642 are arranged in this order. In the wound nonaqueous phase member 601, as long as the separators 641 and 642 are alternately arranged between the electrodes 614 and 615, the disposing position of the separator 642 is not limited to those shown in the drawings. For example, the separator 642 is disposed in contact with the electrode 615 in FIGS. 9A, 9B, and 9C, but the electrode 614 may also be positioned between the separators 641 and 642 by disposing the separator 642 on the right surface of the electrode 614 (i.e., on the right side in each drawing). Note that FIG. 8 does not show the separators 641 and 642.

As shown in FIGS. 9A, 9B, and 9C, the widths of the electrodes 615 and 614 (i.e., the lengths along the spiral shapes after winding) may be the same.

As shown in FIGS. 9A, 93, and 9C, in the nonaqueous phase member 601 which is not wound, the first, second, and third nonaqueous phases 612, 622, and 632 are arranged in this order along the winding direction of the nonaqueous phase member 601. When manufacturing the nonaqueous phase member 601 having, e.g., the configuration shown in FIG. 9A, the nonaqueous phases may be disposed by disposing materials (ionic liquids, enzyme bodies, and mediators) of the nonaqueous phase onto a porous membrane stack obtained by stacking the electrode 614 and separator 641, by a method such as spraying, coating, evacuation, casting, or impregnation. The nonaqueous phase member 601 configured as shown in FIG. 9A may be obtained by sequentially stacking the electrode 615 and separator 642 on the nonaqueous phases formed on the electrode 614. Note that each nonaqueous phase may be gelled or cured as needed by a method to be described later.

Alternatively, when manufacturing the nonaqueous phase member 601 having the configuration shown in FIG. 9B, materials of the nonaqueous phases may be disposed by the above method onto a porous membrane stack obtained by stacking the electrode 614, separator 641, electrode 615, and separator 642.

The stack obtained by stacking the electrode 614, separator 641, electrode 615, and separator 642 may also be impregnated throughout the thickness direction with each of the first, second, and third nonaqueous phases 612, 622, and 632. When obtaining such a configuration, the materials of the nonaqueous phases may be disposed in each of the electrode 614, separator 641, separator 642, and electrode 615 before they are stacked. Alternatively, when winding the porous membrane stack after the nonaqueous phases are disposed on the stack or between the layers of the stack as shown in FIGS. 9A, 9B, and 9C, the nonaqueous phases may be permeated into the separator 641, separator 642, and electrode 615 by appropriately controlling the winding pressure.

In addition, a portion of each of the first, second, and third nonaqueous phases 612, 622, and 632 may impregnate the porous membrane (the electrode 614, separator 641, electrode 615, and separator 642), and another portion thereof may form a layer on the surface of the porous membrane. That is, in the nonaqueous phase member 601 as shown in FIGS. 9A, 9B, and 9C, a portion of each nonaqueous phase may exist on the surface of the porous membrane or between the layers of the porous membrane, while another portion of the nonaqueous phase impregnates the porous membrane. For example, in the configuration shown in FIG. 9C, each of the first, second, and third nonaqueous phases 612, 622, and 632 may have a portion existing between the separator 641 and electrode 615, a portion existing on the surface of the separator 642, and a portion impregnating the electrodes (614 and 615) and separators (641 and 642).

When gelling or curing the nonaqueous phase as described earlier, it is possible to premix a gelling agent, polymeric material, binder, crosslinking agent, support, or the like into the ionic liquid as a material of the nonaqueous phase, in addition to the enzyme body and mediator. By thus mixing the gelling agent, polymeric material, crosslinking agent, binder, or the like, the nonaqueous phase may be gelled or cured after the porous membrane stack is obtained by stacking the electrode 614, separator 641, electrode 615, and separator 642. It is also possible to gel or cure the nonaqueous phase after winding.

As shown in, e.g., FIG. 9C, when the nonaqueous phases (612, 622, and 632) exist as layers between the electrodes 614 and 615 and the layers have a sufficient thickness, the electrodes 614 and 615 do not come in contact with each other even if the separators (641 and 642) are not disposed on the electrode surfaces, so the separators (641 and 642) may be omitted.

The first, second, and third nonaqueous phases 612, 622, and 632 may contain enzyme bodies containing the same one or more kinds of enzymes, or may contain enzyme bodies containing different enzymes. When the first, second, and third nonaqueous phases 612, 622, and 632 contain different enzyme bodies, i.e., when enzymatic reactions in these nonaqueous phases are different, the following measure is desirably taken.

To reliably perform the enzymatic reaction in the first nonaqueous phase 612, the width of a portion where the first nonaqueous phase 612 is disposed is desirably set such that in the porous membrane stack, after the portion including the first nonaqueous phase 612 encircles a layer of a portion including the second nonaqueous phase 622, a part of the portion including the first nonaqueous phase 612 is also covered. That is, the layer including the first nonaqueous phase 612 desirably partially overlap.

Similarly, to reliably perform the enzymatic reaction in the second nonaqueous phase 622, the width of a portion where the second nonaqueous phase 622 is formed is desirably set such that in the porous membrane stack, after the portion including the second nonaqueous phase 622 encircles a layer of a portion including the third nonaqueous phase 632, a part of the portion including the second nonaqueous phase 622 is also covered. That is, the layer including the second nonaqueous phase 622 desirably partially overlap.

The individual reaction cascades of the cascade enzymatic reaction can reliably be performed in order by taking measures as described above, in which the different enzyme bodies contained in the nonaqueous phases are so supported or dispersed as to be arranged along the radial direction of the wound shape of the nonaqueous phase member 601.

When performing the cascade enzymatic reaction, to efficiently progress the generation of the final product by the cascade enzymatic reaction, it is possible to appropriately adjust the volume (amount) of each nonaqueous phase and the enzyme concentration in the nonaqueous phase in accordance with the activity and catalytic efficiency of the enzyme contained in the nonaqueous phase.

In the carbon dioxide fixation device 600 shown in FIG. 8, carbon dioxide may be supplied to the nonaqueous phase member 601 by letting the gaseous phase 611 flow through the gas flow path positioned outside the periphery of the nonaqueous phase member 601, in the same manner as in FIG. 7. The enzymatic reaction in the nonaqueous phase member 601 reduces carbon dioxide and generates a reaction product. Also, the reaction product may be recovered from the nonaqueous phase member 601 by letting the aqueous phase 633 flow through the liquid flow path in the center of the nonaqueous phase member 601.

The carbon dioxide fixation device shown in FIG. 8 can generate a reaction product such as fuel from carbon dioxide by the cascade enzymatic reaction. For example, methanol can be produced from carbon dioxide by the cascade enzymatic reaction in the same manner as in FIG. 7.

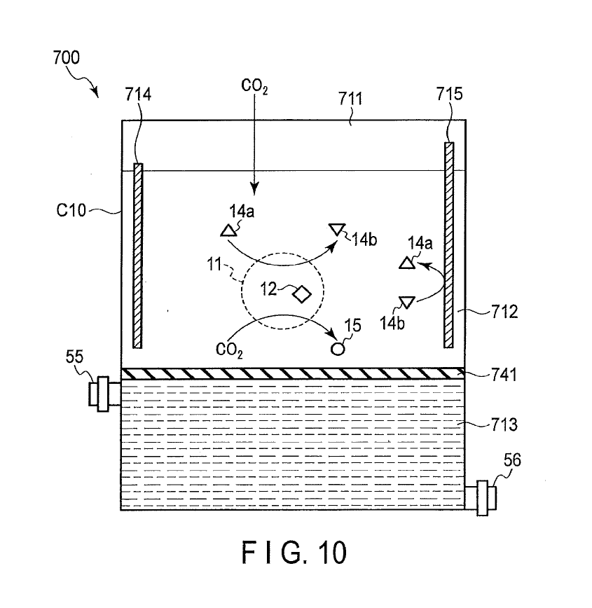

FIG. 10 is a schematic view of a carbon dioxide fixation device of still another example of the first mode according to the embodiment.

Unlike the flow-type devices shown in FIGS. 1, 2, 3, 4, 5, 6, 7, and 8, a carbon dioxide fixation device 700 shown in FIG. 10 is an example of a batch-type device. The carbon dioxide fixation device 700 includes a gaseous phase 711, a nonaqueous phase 712, and an aqueous phase 713. These phases are accommodated in a cell member C10.

The gaseous phase 711 contains a gas containing carbon dioxide. Referring to FIG. 10, a compartment for accommodating the gaseous phase 711 is provided in the cell member C10. This compartment for accommodating the gaseous phase 711 need not be provided in the cell member C10. For example, it is possible to expose the nonaqueous phase 712 to gas containing carbon dioxide such as the atmosphere, and use this gas as the gaseous phase 711. Note that when providing the compartment for accommodating the gaseous phase 711 in the cell member C10, a gas inlet and vent hole communicating with this compartment are desirably provided in the cell member C10, so that the gas containing carbon dioxide can be supplied to the gaseous phase 711.

Like the first block 110 in the device shown in FIG. 1, the nonaqueous phase 712 contains an ionic liquid, an enzyme body 11, and mediators (a reductant 14a and an oxidant 14b). The enzyme body 11 shown in FIG. 10 contains an enzyme 12.

The nonaqueous phase 712 further includes a cathode electrode 715 which reduces the mediator oxidant 14b to the mediator reductant 14a, an anode electrode 714, and a cell voltage control section (not shown) which controls voltage (or potential) applied to the cathode electrode 715 and anode electrode 714.

Instead of the cell voltage control section, it is also possible to use an electric current control section configured to control an electric current which flows between the anode electrode 714 and cathode electrode 715. Alternatively, an electrical control section which can function as both the cell voltage control section and electric current control section may be used.

Note that as long as the cathode electrode 715 and anode electrode 714 are in contact with the nonaqueous phase 712 without coming in contact with each other to become electrically short-circuited, the layout is not limited to that shown in FIG. 10. For example, these electrodes may alternatively be installed at the interface between the gaseous phase 711 and nonaqueous phase 712, or at the interface between the nonaqueous phase 712 and aqueous phase 713. When installing the cathode electrode 715 and/or the anode electrode 714 at the interface between different phases, a porous electrode material is desirably used as the cathode electrode 715 and/or the anode electrode 714.

Also, the nonaqueous phase 712 as an ionic liquid has specific gravity larger than that of the aqueous phase. When using the ionic liquid as is, therefore, a porous support membrane or porous substrate may be disposed between the nonaqueous phase 712 and aqueous phase 713. Furthermore, a separator 741 may be installed singly or in addition to these members.

The aqueous phase 713 contains an extraction liquid which extracts a first reaction product 15 from the nonaqueous phase 712. Also, it is favorable to install a mechanism (not shown) for mechanically stirring the extraction liquid.

Like the first block 110 of the carbon dioxide fixation device 100 as shown in FIG. 1, in the carbon dioxide fixation device 700 shown in FIG. 10, the action of the enzyme 12 reduces carbon dioxide supplied from the gaseous phase 711 to the nonaqueous phase 712, thereby generating the first reaction product 15. The first reaction product 15 is supplied to the aqueous phase 713.

After the concentration of the first reaction product 15 reaches a predetermined value, the aqueous phase 713 may be recovered from, e.g., an outlet provided in the lower portion of the cell member C10.

The enzymatic reaction of reducing carbon dioxide in the nonaqueous phase 712 may be either a single-cascade enzymatic reaction or cascade enzymatic reaction.

When performing the single-cascade reaction, formic acid is generated as the first reaction product 15 by using formate dehydrogenase as the enzyme 12, for example.

When performing the cascade reaction, a plurality of different enzyme bodies are contained in the nonaqueous phase 712. For example, when generating methanol by reducing carbon dioxide by a three-cascade reaction, a first enzyme body containing formate dehydrogenase, a second enzyme body containing formaldehyde dehydrogenase, and a third enzyme body containing alcohol dehydrogenase are contained in the nonaqueous phase 712.

In this example, the first enzyme body reduces carbon dioxide supplied to the nonaqueous phase 712, thereby generating formic acid. This formic acid rapidly migrates to the second enzyme body, and is reduced to formaldehyde. Likewise, the formaldehyde rapidly migrates to the third enzyme body, and is reduced to methanol as a final product.

When performing the cascade reaction by using a plurality of different enzyme bodies, the extraction liquid in the aqueous phase 713 is preferably adjusted so as to be able to selectively extract the final product. In the above example, the extraction liquid is preferably adjusted so as to selectively supply methanol to the aqueous phase 713.

Each of the first, second, and third blocks 110, 120, and 130 of the carbon dioxide fixation device 100 shown in FIG. 1 may be replaced with the carbon dioxide fixation device 700 shown in FIG. 10. In this case, an enzyme body to be contained in the nonaqueous phase 712 is appropriately selected in accordance with an enzymatic reaction to be performed in each block.

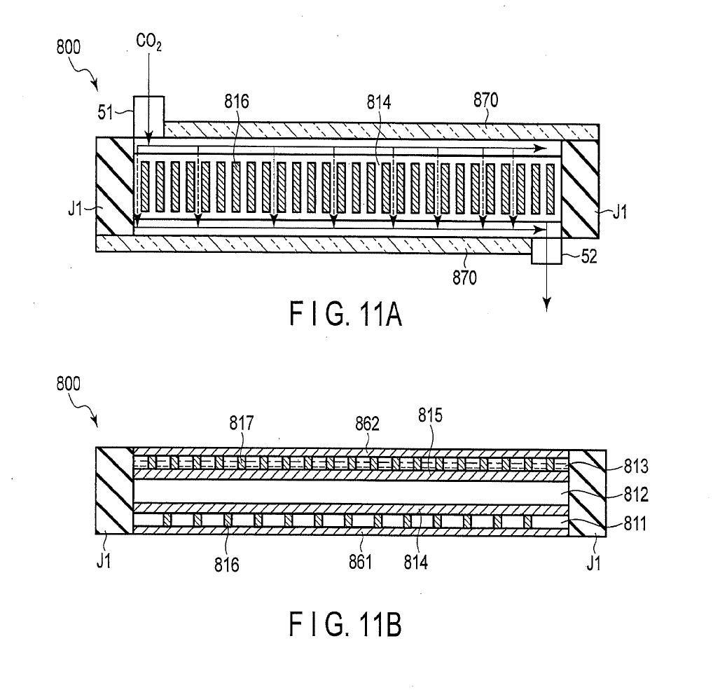

FIGS. 11A and 11B are schematic views of a single-cell for use in a stacked carbon dioxide fixation device as an example according to an embodiment.

FIG. 11A is a schematic plan sectional view showing a single-cell carbon dioxide fixation device 800 from below. FIG. 11B is a schematic side sectional view of the single-cell carbon dioxide fixation device 800.

In the single-cell carbon dioxide fixation device 800 shown in FIGS. 11A and 11B, a gaseous phase 811, an electrode 814 (an anode electrode), a nonaqueous phase 812, an electrode 815 (a cathode electrode), and an aqueous phase 813 are arranged in this order from below. In the shown example, the phases and electrodes each have a planar shape.

The electrode 814 is a porous electrode, and carbon paper may be used as the electrode 814, for example. The electrode 814 is connected to an electrode plate 861 which functions as a flow path for the gaseous phase 811 and also as an electrically conductive lead line. In the example shown in the drawings, multiple partitions that define the flow path of the gaseous phase 811 are provided in between the electrode 814 and electrode plate 861. The partitions may alternatively be omitted.

As shown in FIG. 11A, when a gas containing carbon dioxide is introduced from a gas supply port 51 into the flow path of the gaseous phase 811, this gas flows along the plane through the gap between the electrode plate 861 and electrode 814, and is exhausted from a gas exhaust port 52. When carbon dioxide passes through the gap between the electrode plate 861 and electrode 814, at least a portion of carbon dioxide is further passed through the electrode 814, supplied to the nonaqueous phase 812, and used as a substrate of the enzymatic reaction in the nonaqueous phase 812.

An ionic liquid or the like capable of selectively absorbing carbon dioxide is preferably used as a medium of the nonaqueous phase 812. In this case, gas not absorbed by the nonaqueous phase 812, e.g., gas other than carbon dioxide, is exhausted from the gas exhaust port 52.

The electrode 815 is a porous electrode, and carbon paper may be used as the electrode 815, for example. As another example, the electrode 815 is a sheet-shaped electrode in which multiple slits are formed, or a porous electrode. The electrode 815 is connected to an electrode plate 862 which functions as a flow path of the aqueous phase 813 and also as an electrically conductive lead line.

Like the flow of the gas containing carbon dioxide shown in FIG. 11A, when the extraction liquid is introduced from a liquid inlet (not shown) into the flow path of the aqueous phase 813, this extraction liquid flows along the plane through the gap between the electrode plate 862 and electrode 815, and is let out from the liquid outlet.

Carbon dioxide supplied from the gaseous phase 811 to the nonaqueous phase 812 is reduced by the enzymatic reaction, and a reaction product is generated. This reaction product is supplied to the aqueous phase 813 and recovered.

Furthermore, it is also possible to install a temperature sensor in the single cell (the carbon dioxide fixation device 800), or dispose heat insulating materials 870 on the outside of the single-cell. This makes it possible to measure the temperature of the single-cell by the temperature sensor, and based on the measurement result, control the temperature by a temperature control mechanism or the like so as to obtain conditions appropriate for the enzymatic reaction. In addition, the enzymatic reaction may be performed under more stable temperature conditions by disposing the heat insulating materials 870.

FIG. 11A shows the example in which the heat insulating materials 870 are disposed. The shape of the heat insulating materials 870 is not particularly limited, and the heat insulating materials 870 may have, e.g., a slab-shaped form. The heat insulating materials 870 may also be omitted.

Each of the phases and electrodes are integrated by a jig J1. To prevent electrical short-circuits, the jig J1 may be made of an electrically insulating material, for example. Alternatively, when using a conductive material such as a metal or alloy as the jig J1 by taking account of properties such as strength, it is desirable to prevent electrical short-circuits by measures such as forming an insulating film on the surface of the jig J1.

The above-described partitions in the gaseous phase 811 are one example of the spacers 816 shown. The form of the spacers 816 are not limited to those like the depicted partitions. The spacers 816 may have a form other than partitions. For example, the spacers 816 may serve a function of preventing deformation of the electrode 814 caused by stress such as pressure; however, the spacers 816 may be omitted if the electrode 814 has sufficient strength.

The spacers 816 may be formed from an electrically conductive material or the like. In this case, the electrode 814 and electrode plate 861 may electrically be connected via the spacers 816. Alternatively, it is also possible to use an electrode plate having been processed to have formed projections as the electrode plate 861, and use the projections in place of the spacers 816. In this case, the electrode 814 and electrode plate 861 are directly electrically connected via the projections.

On the other hand, electrically conductive leads or the like for electrical connection may additionally be provided, instead of electrically connecting the electrode 814 and electrode plate 861 via the spacers 816. In this case, an electrically insulating material is desirably used as the material of the spacers 816. It is also desirable to appropriately select the material of the spacers 816 in accordance with the components and temperature of gas supplied to the gaseous phase 811.

Examples of the material usable as the spacers 816 are highly electrically conductive materials such as stainless steel, nickel, copper, aluminum, tungsten, an electrically conductive polymer, and graphite. Examples of other materials are electrically nonconductive materials such as plastic, ceramics, and glass.

By defining the flow path in the gaseous phase 811 by the spacers 816, it is possible to sufficiently prolong the time during which the flowing gas flows along the surface of the electrode 814. For example, the amount of carbon dioxide supplied from the flowing gas to the nonaqueous phase 812 through the electrode 814 can be increased by installing the spacers 816 like partitions so as to prolong the flow path from the gas supply port 51 to the gas exhaust port 52. The shape and layout of the spacers 816 are not limited to those shown in the drawings, as long as the flow path through which carbon dioxide can be supplied to the nonaqueous phase 812 can be secured. For example, the shape of the spacer 816 may be a dot shape instead of the slit shape. It is desirable to appropriately design the shape and layout of the spacers 816 in accordance with the state of a supply source of the gas introduced to the gas supply port 51, and the conditions such as the flow velocity, temperature, and pressure of the introduced gas.

In a similar manner for the aqueous phase 813, multiple spacers 817 for defining an extraction liquid flow path may be formed between the electrode 815 and electrode plate 862. The spacers 817 may be formed from an electro conductive material or the like. In this case, the electrode 815 and electrode plate 862 may be electrically connected via the spacers 817. Alternatively, it is also possible to use an electrode plate having processed to have formed projections as the electrode plate 862, and use the projections in place of the spacers 817. In this case, the electrode 815 and electrode plate 862 are directly electrically connected via the projections.

On the other hand, electrically conductive leads or the like for electrical connection may additionally be disposed, instead of electrically connecting the electrode 815 and electrode plate 862 via the spacers 817. In this case, an electrically insulating material is desirably used as the material of the spacers 817. It is also desirable to appropriately select the material of the spacers 817 in accordance with the components and the like of the extraction liquid.

Examples of the material usable as the spacers 817 are highly electrically conductive materials such as stainless steel, nickel, copper, aluminum, tungsten, an electrically conductive polymer, and graphite. Examples of other materials are electrically nonconductive materials such as plastic, ceramics, and glass.

By defining the flow path in the aqueous phase 813 by the spacers 817, it is possible to sufficiently prolong the time during which the flowing extraction liquid flows along the surface of the electrode 815. For example, it is possible to increase the amount of the reaction product extracted from the nonaqueous phase 812 by the extraction liquid, by installing the spacers 817 like partitions so as to prolong the flow path from the liquid inlet to the liquid outlet. The shape and layout of the spacers 817 are not limited, as long as the flow path through which the reaction product can be extracted from the nonaqueous phase 812 can be secured. For example, the shape of the spacers 817 may be a slit shape or dot shape. It is desirable to appropriately design the shape and layout of the spacers 817 in accordance with the conditions such as the flow velocity, temperature, and pressure of the extraction liquid introduced into the liquid inlet.

Note that the spacers 817 may serve a function of preventing deformation of the electrode 815 caused by stress such as pressure. However, the spacers 817 may be omitted if the electrode 815 has sufficient strength. On the other hand, if the spacers 817 are omitted, the flow of the extraction liquid becomes difficult to control because there is no extraction liquid flow path. Accordingly, it is favorable to define an extraction liquid flow path in the aqueous phase 813 by forming the spacers 817.

Gaskets made of a material such as resin may also be formed at, e.g., the end portions of the electrode plates 861 and 862. The gaskets can prevent a leak of carbon dioxide or the extraction liquid from within the single-cell.

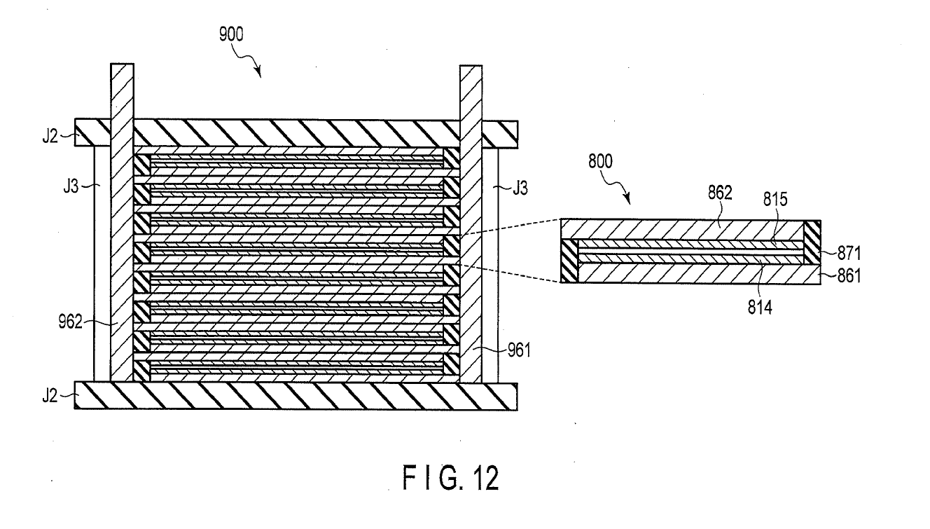

A stacked carbon dioxide fixation device 900 shown in FIG. 12 is obtained by stacking the single-cell carbon dioxide fixation device 800 shown in FIGS. 11A and 11B. Note that in FIG. 12 the carbon dioxide fixation device 800 shown in FIGS. 11A and 11B are depicted having been simplified. Note that in FIG. 12, reference numeral 871 indicates a gasket.

The stacked carbon dioxide fixation device 900 shown in FIG. 12 includes a plurality of single-cells (the carbon dioxide fixation devices 800). These single-cells compose a stacked structure by being stacked in the thickness direction, and are integrated by jigs J2 and J3. An electrical insulating sheet (not shown) is formed between adjacent single-cells in order to prevent an electrical contact between the electrode plate 861 of one single cell and the electrode plate 862 of another single cell.

The jigs J2 are arranged at the two ends of the stacked structure in the stacking direction, and the plurality of stacked single-cells are arranged therebetween. In this state, the single-cells are integrated by connecting the jigs J2 at the two ends by the jigs J3 arranged along the stacking direction. The jig J2 may be a planar member, for example. The jig J3 may be a columnar member, for example. The jigs J2 and J3 may be made of an electrically insulating material in order to prevent electrical short-circuits. Alternatively, when using a conductive material such as a metal or alloy as the jigs J2 and J3 by taking account of properties such as strength, it is desirable to prevent electrical short-circuits by forming an insulating film or the like on the surfaces of the jigs J2 and J3.

The gas supply port 51 and gas exhaust port 52 of each single-cell are connected to a common gas supply path and common gas exhaust path (neither is shown). It is also possible to connect the gas exhaust port 52 of one single-cell to the gas supply port 51 of another single-cell, thereby forming gas flow paths connected in series. By thus changing the gas flow path connecting method, it is possible to simultaneously supply the gaseous phase to all the single-cells, or sequentially supply the gaseous phase to each single-cell.

Also, the liquid inlet and liquid outlet of each single-cell are connected to a common liquid inlet path and common liquid outlet path (neither is shown). Alternatively, it is also possible to connect the liquid outlet of one single-cell to the liquid inlet of another single-cell, thereby forming liquid flow paths connected in series. By thus changing the liquid flow path connecting method, it is possible to simultaneously supply the extraction liquid (aqueous phase) to all the single-cells, or sequentially supply the extraction liquid (aqueous phase) to each single-cell.

In the stack including these single-cells, an electrode lead line 961 electrically connects the electrode plates 861 of the single-cells. Similarly, an electrode lead line 962 electrically connects the electrode plates 862 of the single-cells. The electrode lead lines 961 and 962 are electrically connected to a cell voltage control section. When the cell voltage control section applies voltage between the electrode lead lines 961 and 962, the voltage is applied between the electrode plates 861 and 862 of each single cell.

The electrode lead lines 961 and 962 may alternatively be electrically connected to an electric current control section, instead of the cell voltage control section. The electric current control section can control, e.g., an electric current which flows between the electrode plates 861 and 862 of each single-cell. It is alternatively possible to use an electrical control section capable of functioning as both the cell voltage control section and electric current control section.

Referring to FIG. 12, the electrode plates 861 of the single-cells are electrically connected in parallel. However, the electrode plates 861 may alternatively be electrically connected in series. Likewise, the electrode plates 862 are electrically connected in parallel in FIG. 12, but the electrode plates 862 may alternatively be electrically connected in series.

The device having this configuration can efficiently fix carbon dioxide.

In the examples of the carbon dioxide fixation device shown in the drawings, the form in which the mediator is regenerated by an electrochemical method has been explained in detail. When regenerating the mediator by a method different from the electrochemical method, the cathode electrode and anode electrode may be omitted. Alternatively, a cathode electrode which also functions as a porous membrane and an anode electrode which also functions as a porous membrane may be replaced with porous membranes or spacers which do not function as electrodes. When the cathode electrode and anode electrode are omitted or replaced, the electrical control section may be omitted.

Also, different mediator regenerating methods may be used among the blocks (e.g., the first, second, and third blocks 110, 120, and 130 shown in FIG. 1). For example, it is possible to adopt the electrochemical method in the first and second blocks, and an enzymatic method in the third block. In this case, each of the first and second blocks includes a cathode electrode and anode electrode in contact with the nonaqueous phase. The third block includes an enzyme body which catalyzes the enzymatic reaction of regenerating the mediator. The cathode electrode and anode electrode of the third block may be omitted or replaced.

The carbon dioxide fixation device according to the first embodiment includes the nonaqueous phase which includes the ionic liquid, the enzyme body which catalyzes a reduction reaction of carbon dioxide or a reduced product thereof, and the mediator which acts as a reducing agent or coenzyme in the reduction reaction. To the nonaqueous phase is supplied carbon dioxide or a reduced product thereof, and the nonaqueous phase generates a reaction product by the reduction reaction. The device also includes the aqueous phase which contains an extraction liquid containing water, and to which the reaction product is supplied from the nonaqueous phase. The device having this configuration can efficiently fix carbon dioxide.

### Second Embodiment

The second embodiment provides a fuel production system including a carbon dioxide supply section, a fuel generation section, and an extraction liquid recovery section.

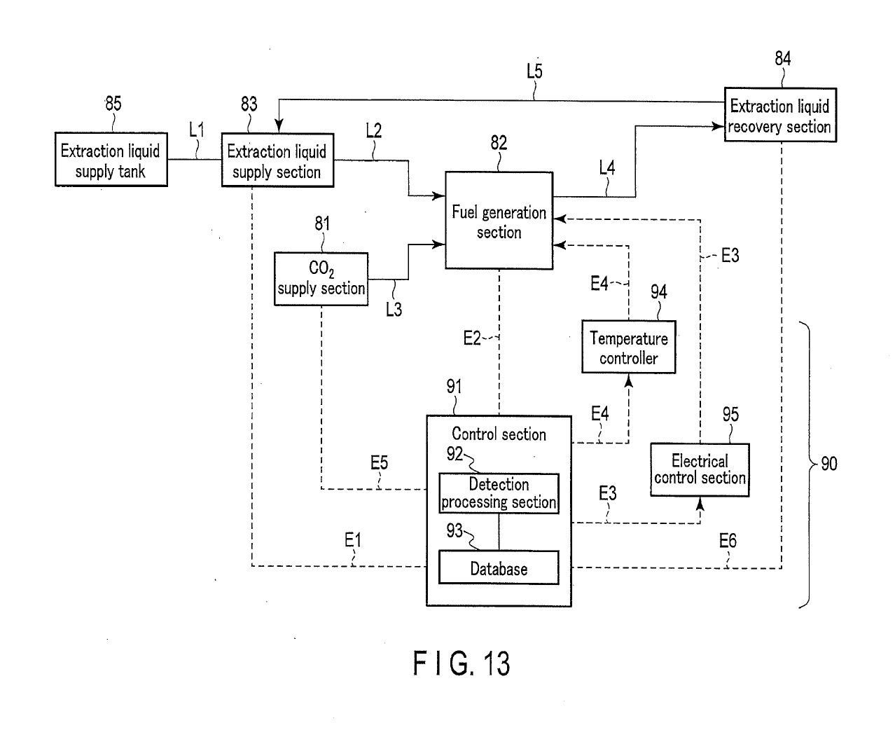

FIG. 13 shows an example of the fuel production system according to the second embodiment.

The production of fuel from carbon dioxide can automatically be controlled by installing the carbon dioxide fixation device according to the first embodiment in the fuel generation section of the system shown in FIG. 13.

The dotted lines (external electric lines E1 to E6) shown in FIG. 13 indicate an electrical system circuit. The solid lines (L1 to L5) shown in FIG. 13 indicate a gaseous phase or liquid phase piping system.

The circuit lines (E1 to E6) in the electrical system circuit function as external electric lines for supplying power, and also function as external signal lines for transmitting signals such as electric signals for controlling each of the members.

To externally apply voltage to the carbon dioxide fixation device installed in a fuel generation section 82, a control section 91 and a cell voltage control section are connected via the external electric lines E2 and E3. Also, the control section 91 and a temperature controller 94 are connected to the fuel generation section 82 via the external electric lines E2 and E4.

The control section 91 within an electrical system 90 is configured to designate, to the cell voltage control section, the conditions of the voltage to be applied to the carbon dioxide fixation device in accordance with, e.g., temperature condition information from a sensor installed in the carbon dioxide fixation device, and carbon dioxide supply information of a carbon dioxide supply section 81. In addition, the control section 91 in the electrical system 90 is configured to designate the conditions of the supply flow velocity of an extraction liquid to an extraction liquid supply section 83, in accordance with, e.g., the carbon dioxide supply information of the carbon dioxide supply section 81. Also, the control section 91 is configured to instruct the temperature controller 94 to control the temperature of the fuel generation section 82.

The cell voltage control section may be replaced with an electric current control section. In this case, the control section 91 is configured to designate the conditions of an electric current to be flown in the carbon dioxide fixation device.

The shown electrical control section 95 may be a cell voltage control section configured to control voltage to be applied to the carbon dioxide fixation device. Alternatively, the electrical control section 95 may be an electric current control section configured to control electric current in the carbon dioxide fixation device. Furthermore, the electrical control section 95 may be equipped with a mode for functioning as the cell voltage control section and a mode for functioning as the current control section. The electrical control section 95 may be configured to switch operating modes between the mode as cell voltage control section and mode as electric current control section, based on instructions from the control section 91.

The carbon dioxide supply section 81 may also be configured to have a function of controlling the pressure of a gas containing carbon dioxide. For example, the control section 91 detects the gas pressure in the carbon dioxide supply section 81, and instructs pressure control via the external electric line E5 (an external signal line) as needed.

An extraction liquid recovery section 84 recovers fuel from the extraction liquid, and adjusts the pH of the extraction liquid. After that, the extraction liquid is supplied to the extraction liquid supply section 83 and reused.

The extraction liquid supply section 83 is configured to temporarily store the extraction liquid supplied from the extraction liquid recovery section 84, and to supply the extraction liquid to the fuel generation section 82 at a predetermined flow velocity in accordance with a command from the control section 91.

Furthermore, it is possible to change and adjust conditions of the extraction liquid at the extraction liquid supply section 83 by using an extraction liquid supply tank 85.

The fuel production system according to the second embodiment includes the fuel generation section including the carbon dioxide fixation device according to the first embodiment. Therefore, this system can efficiently fix carbon dioxide.

### EXAMPLES

Examples will be explained below as practical design examples of the carbon dioxide fixation device according to the embodiment.

### Example 1

A carbon dioxide fixation device of Example 1 is a carbon dioxide fixation device including first, second, and third blocks, like the carbon dioxide fixation device 100 shown in FIG. 1. Also, the carbon dioxide fixation device of Example 1 produces methanol as fuel from carbon dioxide, as an example of a reaction of fixing carbon dioxide.

In Example 1, methanol is produced by the following three-cascade enzymatic reaction:

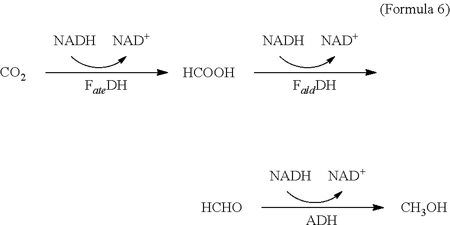

The carbon dioxide fixation device of Example 1 will be explained below, assuming the device to have the same configuration as that of the carbon dioxide fixation device 100 shown in FIG. 1.

The first enzymatic reaction is a reaction of generating formic acid or formate from carbon dioxide as a substrate by using formate dehydrogenase (FₐₜₑDH:FDH). The first enzymatic reaction is performed in, e.g., the nonaqueous phase 112 in the first block 110 shown in FIG. 1.

The second enzymatic reaction is performed in the second block 120, and generates formaldehyde in the presence of formaldehyde dehydrogenase (F_{ald}DH) as an enzyme by using formic acid or formate generated in the first block 110 as a substrate.

The third enzymatic reaction is performed in the third block 130, and generates methanol by an enzymatic reaction in the presence of alcohol dehydrogenase (ADH) by using formaldehyde generated in the second block 120 as a substrate.

An ion gel made of 1-ethyl-3-methylimidazolium bis(trifluoromethylsulfonyl)amide (abbreviation: EMIMTFSI) was used for the nonaqueous phase 112 of the first block 110.

The enzyme body 11 taking part in the first enzymatic reaction was obtained by impregnating a silica gel, in which formate dehydrogenase (FₐₜₑDH) was dispersed and immobilized, with 3 mM NADH and a 0.1 M phosphate buffer solution having a pH of 7. The silica gel used herein was made of porous spherical silica particles having mesopores, and had an average particle size of 0.3 μM and an average pore size of 16 nm. The obtained enzyme body 11 was dispersed in EMIMTFSI as a medium of the first nonaqueous phase.

EMIMTFSI containing the FₐₜₑDH/silica gel as the enzyme body 11 was further made into a tetra-armed poly(ethylene glycol) ion gel. The FₐₜₑDH/silica gel was evenly dispersed among the mesh of the ion gel of EMIMTFSI. Thus, an ion gel membrane containing the enzyme body 11 was obtained as the nonaqueous phase 112 of the first block 110.

This ion gel membrane of EMIMTFSI containing the enzyme body 11 was sandwiched between carbon papers as a pair of porous electrodes. The carbon papers were modified by poly(neutral red). In addition, in the first block 110, one porous electrode (the anode electrode 114) was placed in contact with the carbon dioxide flow path side, and the other porous electrode (the cathode electrode 115) was placed in contact with the aqueous phase side.

A phosphate buffer solution having a pH of 7 and a concentration of 50 mM was used as a fuel extraction liquid contained in the aqueous phase 113 of the first block 110. Fuel generated by the enzymatic reaction in the nonaqueous phase 112 containing the enzyme body 11 becomes extracted to the aqueous phase 113.

An ion gel containing the enzyme body 21 containing formaldehyde dehydrogenase (F_{ald}DH) as an enzyme was used as the nonaqueous phase 122 taking part in the second enzymatic reaction. An ion gel in which the enzyme body 21 was dispersed was obtained as follows.

For the ionic liquid as precursor of the ion gel, a solution mixture (χ_{PIL}=AIL/PIL=0.7) of [C₈mIm⁺][TFSA⁻] as AIL and [C₈ImH⁺][TFSA⁻] as PIL was used. [C₈mIm⁺][TFSA⁻] is a hydrophobic ionic liquid, and [C₈ImH⁺][TFSA⁻] is a hydrophilic ionic liquid. [C₈ImH⁺][TFSA⁻] also functions as a cosurfactant.

AOT was added to this solution mixture, and AOT (0.07 M) was dispersed by stirring the solution mixture for 20 hours. Subsequently, a dilute buffer solution [50 mM phosphoric acid buffer, pH=7] including F_{ald}DH as the enzyme and 3 mM NADH as the mediator was added as an aqueous medium, and the solution mixture was stirred for 1 hour. Thereby, a reversed micelle made of AOT and [C₈ImH⁺][TFSA⁻] including a water pool was prepared within the solution mixture of [C₈mIm⁺][TFSA⁻] and [C₈ImH⁺][TFSA⁻] as the nonaqueous phase. F_{ald}DH is solubilized as the enzyme in the water pool of the thus obtained reversed micelle (the second enzyme body).

Then, the ionic liquid solution mixture was set at a temperature of 40° C. to 50° C. in a state in which the enzyme bodies are dispersed, and an appropriate amount of a gelatin powder was added to the solution mixture. After that, the solution mixture was vigorously stirred for about 30 min. Subsequently, the solution mixture was cooled to 30° C. while stirring, and kept stirring until the solution became very thick and uniform. The obtained suspension was left to stand at room temperature until the solution had become a transparent gel.

In the abovementioned treatment process, gelatin enters the water pool of the enzyme body (the reversed micelle), and gels there. Furthermore, since gelatin having gelled in the water pool forms an intermolecular network, the entire nonaqueous phase including the enzyme bodies gels. In addition, since the suspension is left to stand at room temperature, refolding of proteins (gelatin and F_{ald}DH) that had been thermally denatured by heating can be further performed.

As the enzyme body relevant to the third enzymatic reaction (enzyme body 31), a reversed micelle including the enzyme alcohol dehydrogenase (ADH) was used. This enzyme body was dispersed in a solution mixture (χ_{PIL}=AIL/PIL=0.4) of [C₈mIm⁺][TFSA⁻] as an aprotic ionic liquid (AIL) and [C₄ImH⁺][TFSA⁻] as a protic ionic liquid (PIL). [C₈mIm⁺][TFSA⁻] is a hydrophobic ionic liquid, and [C₄ImH⁺][TFSA⁻] is a hydrophilic ionic liquid. [C₄ImH⁺][TFSA⁻] functions also as a cosurfactant.

The nonaqueous phase 132 containing the enzyme body 31 was obtained as follows.

Sodium 1,2-bis(2-ethylhexylcarbonyl)-1-ethanesulfonate (Aerosol OT: AOT) as an anionic surfactant was added to a solution mixture of [C₈mIm⁺][TFSA⁻] and [C₄ImH⁺][TFSA⁻], and AOT (0.07 M) was dispersed by stirring the mixture for 20 hrs. Subsequently, a dilute buffer solution [50 M phosphoric acid, pH=7] (0.02 M PBS) containing alcohol dehydrogenase (ADH) as an enzyme and NADH as a mediator was added as an aqueous solution, and the mixture was stirred for 1 hr, thereby a reversed micelle made of AOT and [C₄ImH⁺][TFSA⁻] and including a water pool containing an enzyme body was formed in the solution mixture of [C₈mIm⁺][TFSA⁻] and [C₄ImH⁺][TFSA⁻] as a medium of the nonaqueous phase.

By the above-mentioned injection method, the reversed micelle in which ADH was solubilized in the water pool was formed. Also, at the same time the reversed micelle as an enzyme body was formed, this reversed micelle was dispersed in the medium of the nonaqueous phase.

Since NADH can be sufficiently supplied to the enzymatic reaction in the water pool of the reversed micelle as the third enzyme body (enzyme body 31) or in the nonaqueous phase, the enzymatic reaction in the third block 130 can smoothly progress. Methanol generated in the enzymatic reaction is extracted to the extraction liquid (aqueous phase) in contact with a porous electrode (the cathode electrode 135).

An anode electrode was placed at the nonaqueous phase of each block. In addition, a reference electrode (not shown) was placed at an appropriate position in the nonaqueous phase of each block.

Methanol is transported to a separating apparatus (not shown) through external circulation piping, and methanol as final fuel is separated and recovered there. After that, the pH of the extraction liquid is adjusted, and the extraction liquid is reintroduced into the aqueous phase 113 of the first block 110 from an inlet communicating with the aqueous phase 113.

NADH is used as the mediator of the three enzymatic reactions described above. NADH is dispersed in each nonaqueous phase and each enzyme body. In the abovementioned three blocks, NADH is oxidized to NAD⁺ by the respective enzymatic reactions. In each block, the oxidized NAD⁺ is reduced to NADH again by the carbon paper (cathode electrode) modified by electropolymerization with poly(neutral red). Note that NADH may be added in a supersaturated state to the nonaqueous phase.

In the carbon dioxide fixation device of Example 1, the concentration of methanol as the final fuel can be raised by raising the pressure (e.g., to about 1.5 MPa) of carbon dioxide to be supplied to the gaseous phase 111 of the first block 110, or by accelerating the supply of carbon dioxide.

Note that proton ions necessary for the three enzymatic reactions described above can be supplied from the extraction liquid.

It was possible to obtain methanol from carbon dioxide at high fuel conversion efficiency by using the carbon dioxide fixation device of Example 1.

### Example 2

A carbon dioxide fixation device of Example 2 is a device for producing methanol as fuel from carbon dioxide by a two-cascade enzymatic reaction.

Like the carbon dioxide fixation device 400 shown in FIG. 6, this carbon dioxide fixation device of Example 2 includes a plurality of adjacent nonaqueous phases. However, FIG. 6 shows three nonaqueous phases (412, 422, and 432) each containing an enzyme body, whereas the carbon dioxide fixation device of Example 2 includes two nonaqueous phases each containing an enzyme body. That is, the number of the nonaqueous phases of the carbon dioxide fixation device of Example 2 is smaller by one than that of the carbon dioxide fixation device 400. Also, the number of electrodes of the carbon dioxide fixation device of Example 2 can be made smaller by one than that of the electrodes (415, 425, and 435) of the carbon dioxide fixation device 400. The carbon dioxide fixation device of Example 2 will be explained below, assuming the device as having a configuration where the third nonaqueous phase 432 and electrode 435 are omitted from FIG. 6.

In Example 2, methanol is produced by the following two-cascade enzymatic reaction:

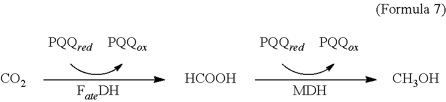

In the above formula, PQQ is pyrroloquinoline quinone. The molecular structure is as follows:

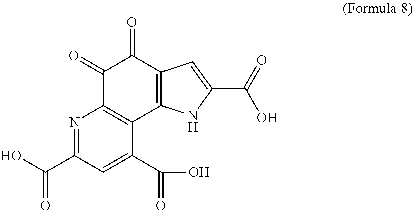 [SMILES: O=C(O)c1cc(C(=O)O)c2c(n1)C(=O)C(=O)c1cc(C(=O)O)[nH]c1-2]

PQQ functions as a mediator in an enzymatic reaction by formate dehydrogenase (FₐₜₑDH) or methanol dehydrogenase (MDH). When participating in the enzymatic reaction, PQQ is oxidized from reduced PQQ_{red} to oxidized PQQₒₓ.

PQQₒₓ is reduced to PQQ_{red} again at the electrode 415 or 425 installed to the nonaqueous phase containing an enzyme body, and participates in the enzymatic reaction.

The first enzymatic reaction of Example 2 is a reaction of generating formic acid from carbon dioxide as a substrate by using formate dehydrogenase (FₐₜₑDH) as an enzyme.

The first enzymatic reaction is performed in the first nonaqueous phase 412.

The second enzymatic reaction is performed in the second nonaqueous phase 422, and produces methanol in the presence of an enzyme (MDH) by using formic acid generated in the first nonaqueous phase as a substrate.

In the carbon dioxide fixation device of Example 2, like that in FIG. 6, the fourth nonaqueous phase 402 containing no enzyme is disposed adjacent to the first nonaqueous phase 412 containing an enzyme. The fourth nonaqueous phase 402 is in contact with the gaseous phase 411 contained in a supply flow path of carbon dioxide. Carbon dioxide introduced into the gaseous phase 411 is absorbed in the fourth nonaqueous phase 402 once, and then introduced into the first nonaqueous phase 412 containing the first enzyme body. Note that the fourth nonaqueous phase 402 may also be omitted.

The electrode 414 is disposed at the fourth nonaqueous phase 402. When omitting the fourth nonaqueous phase 402, the electrode 414 may be installed in contact with the first nonaqueous phase 412 and not in contact with the electrode 415.

To prevent the diffusion of the enzyme body contained in the first nonaqueous phase 412, the separator 441 is disposed between the fourth nonaqueous phase 402 and first nonaqueous phase 412. In addition, the separator 442 is disposed between the second nonaqueous phase 422 and aqueous phase 433.

An ion gel made of 1-ethyl-3-methylimidazolium bis(trifluoromethylsulfonyl)amide (abbreviation: EMIMTFSI) was used as the fourth nonaqueous phase 402. No enzyme body was dispersed in this ion gel.

As the first enzyme body taking part in the first enzymatic reaction, used was a material obtained by further impregnating a silica gel containing formate dehydrogenase (FₐₜₑDH) with 5 mM PQQ and a 0.1 M phosphoric acid buffer solution having a pH of 7. This enzyme body was dispersed in EMIMTFSI as the first nonaqueous phase 412. Then, EMIMTFSI containing the FDH/silica gel as the first enzyme body was further made into a tetra-armed poly(ethylene glycol) ion gel.

As the second enzyme body taking part in the second enzymatic reaction, used was a material obtained by further impregnating a silica gel containing methanol dehydrogenase (MDH) with 5 mM PQQ and a 0.1 M phosphoric acid buffer solution having a pH of 7. This enzyme body was dispersed in EMIMTFSI as the second nonaqueous phase 422.

The carbon dioxide fixation device of Example 2 was manufactured by installing, in the cell member C10, an enzyme membrane electrode fused assembly (enzyme membrane electrode assembly: EMEA) formed by sequentially stacking the fourth nonaqueous phase 402, the separator 441, the electrode 414, the EMIMTFSI ion gel membrane (the first nonaqueous phase 412) containing the first enzyme body, the electrode 415, EMIMTFSI (the second nonaqueous phase 422) containing the second enzyme body, the electrode 425, and the separator 442. Carbon papers were used as the electrodes 415 and 425.

Carbon paper was used as the electrode 414.

This device was able to efficiently manufacture methanol as fuel from carbon dioxide.

### Example 3

Like the carbon dioxide fixation device of Example 2, a carbon dioxide fixation device of Example 3 is a device for producing methanol as fuel from carbon dioxide by a two-cascade enzymatic reaction.

In Example 3, methanol is produced by the two-cascade enzymatic reaction, i.e., enzymatic reactions by formate dehydrogenase (FₐₜₑDH) and methanol dehydrogenase (MDH), as in Example 2. Also, the same mediators, nonaqueous phases, and extraction liquids as those of Example 2 were used in Example 3.

In Example 3, a wound enzyme membrane electrode fused assembly (EMEA) as shown in FIG. 8 was formed, unlike the stack type in Example 2. However, this wound enzyme membrane electrode fused assembly (EMEA) of Example 3 includes a single nonaqueous phase, unlike the nonaqueous phase 601 including the three partitioned nonaqueous phases shown in FIGS. 8, 9A, 9B, and 9C. Also, in Example 3, two enzymes FₐₜₑDH and MDH are contained in the same enzyme body, and this enzyme body is dispersed in the single nonaqueous phase.

Carbon cloth was used as porous electrodes (the electrodes 614 and 615). In addition, a separator was formed on one of the porous electrodes.

An enzyme membrane electrode fused assembly (EMEA) obtained by forming a porous electrode, a nonaqueous phase containing the enzyme body, another porous electrode, and a separator in this order was wound such that the separator was on the outside. Note that a hollow space to be used as an extraction liquid flow path was left remaining in the center of the wound body of this enzyme membrane electrode fused assembly (EMEA).

A porous support member (the inner tube 653) for supporting the enzyme membrane electrode fused assembly (EMEA) was further disposed in the central flow path of the enzyme membrane electrode fused assembly. An extraction liquid for extracting fuel flows through this central flow path.

On the other hand, a porous support member (the intermediate tube 652) for similarly supporting the enzyme membrane electrode fused assembly (EMEA) was further disposed on the outer circumferential surface of the wound body of the enzyme membrane electrode fused assembly (EMEA). Then, the enzyme membrane electrode fused assembly (EMEA) including the support members was installed within an exterior member (the outer tube 651) so as to form a space as a flow path for supplying carbon dioxide.

Carbon dioxide is introduced from the outer-circumference flow path. This carbon dioxide introduced from the outer-circumference flow path reaches the layer of the nonaqueous phase containing the enzyme body, through the porous support member supporting the enzyme membrane electrode fused assembly (EMEA). In this nonaqueous phase containing the enzyme body, carbon dioxide participates in the enzymatic reaction as a substrate of the enzymatic reaction. In the nonaqueous phase, the two-cascade enzymatic reaction started using carbon dioxide as a first substrate is performed, and methanol as fuel is finally generated.

The generated methanol is finally extracted by the extraction liquid flowing through the central flow path, through the porous support member on the inner circumference of the enzyme membrane electrode fused assembly (EMEA).

It was possible to obtain methanol from carbon dioxide at high fuel conversion efficiency by using the carbon dioxide fixation device of Example 3.

### Example 4

A carbon dioxide fixation device of Example 4 is a batch-type carbon dioxide fixation device like the carbon dioxide fixation device 700 shown in FIG. 10, and is a device which produces methanol as fuel from carbon dioxide.

This carbon dioxide fixation device of Example 4 will be explained below, assuming the device as having the same configuration as that of the carbon dioxide fixation device 700 shown in FIG. 10.

In Example 4, methanol is produced by a two-cascade enzymatic reaction using FₐₜₑDH and MDH as in Example 2.

Mediators and fuel extraction liquids in Example 4 are the same as those used in Example 2.

A solution mixture (χ_{PIL}=AIL/PIL=0.7) of ionic liquids [C₈mIm⁺][TFSA⁻] and [C₈ImH⁺][TFSA⁻] was used as a nonaqueous phase of Example 4. AOT was added to this solution mixture, and AOT (0.07 M) was dispersed by stirring the mixture for 20 hrs. Subsequently, a dilute buffer solution [0.1 M phosphoric acid buffer solution, pH=7.0] containing FₐₜₑDH and MDH as enzymes was added as an aqueous solvent, and the mixture was stirred for 1 hr, thereby forming a reversed micelle made of AOT and [C₈ImH⁺][TFSA⁻] and including a water pool, in the solution mixture of [C₈mIm⁺][TFSA⁻] and [C₈ImH⁺][TFSA⁻] as a nonaqueous phase.

FₐₜₑDH and MDH are solubilized as enzymes in the water pool of the reversed micelle obtained as described above. In Example 4, this reversed micelle in which FₐₜₑDH and MDH were immobilized was used as an enzyme body.

The nonaqueous phase containing the enzymes/reversed micelle/ionic liquid obtained as described above is used as a nonaqueous phase of Example 4, i.e., as the nonaqueous phase 712 of the batch-type carbon dioxide fixation device like that shown in FIG. 10.

The cathode electrode 715 and anode electrode 714 are disposed at the nonaqueous phase. Although not shown in FIG. 10, the carbon dioxide fixation device of Example 4 further includes a reference electrode (not shown) disposed at the nonaqueous phase 712.

The separator 741 and a porous support (not shown) are disposed between the nonaqueous phase and an aqueous phase containing a fuel extraction liquid.

The extraction liquid inlet 55 and extraction liquid outlet 56, both of which communicate with the aqueous phase 713, are disposed. Although not shown in FIG. 10, a stirrer is placed in the aqueous phase 713 of the carbon dioxide fixation device of Example 4. The extraction liquid is stirred by rotating the stirrer by a stirring apparatus provided outside.

Carbon dioxide is introduced from a gaseous phase in contact with the nonaqueous phase, and participates in an enzymatic reaction as a substrate for the enzyme FₐₜₑDH dispersed in the nonaqueous phase. As in Example 2, formic acid or formate generated by the enzymatic reaction catalyzed by FₐₜₑDH is used as a substrate of the next enzymatic reaction catalyzed by the enzyme MDH, and methanol is finally generated. Methanol is extracted to the extraction liquid contained in the aqueous phase through the separator 741 and a porous support. After the methanol concentration in the extraction liquid has risen as the reaction time passes and has reached a predetermined concentration, the extraction liquid containing methanol is recovered from the extraction liquid outlet 56 communicating with the aqueous phase. Then, a new extraction liquid is let in from the extraction liquid inlet 55 communicating with the aqueous phase, and the above process is repeated.

The fuel production method of Example 4 is a batch-type fuel generation method, and can produce methanol of high concentration.

In Example 4, the enzymes are solubilized in the water pool of the reversed micelle, and hence can evenly be dispersed within the reaction system (ionic liquid). Thereby, a high enzyme activity and high enzymatic catalytic efficiency can be obtained.

It was possible to obtain methanol from carbon dioxide at high fuel conversion efficiency by using the carbon dioxide fixation device of Example 4.

### Example 5

A carbon dioxide fixation device of Example 5 is a batch-type carbon dioxide fixation device and produces methanol as fuel from carbon dioxide, like the device of Example 4.

In Example 5, methanol is produced by a two-cascade enzymatic reaction using FₐₜₑDH and MDH as in Example 2.

The same mediators and fuel extraction liquids as those used in Example 2 are used in Example 5.

In Example 5, enzymes/reversed micelle/ionic liquid were formed by the same method as in Example 4, and gelled by the following method.

A proper amount of gelatin powder was added to a mixture (a nonaqueous phase containing the enzymes/reversed micelle/ionic liquid) in which an enzyme body containing the enzymes/reversed micelle was dispersed, and the mixture was vigorously stirred at a temperature of 40° C. to 50° C. for about 30 min. Subsequently, the solution mixture was cooled to 30° C. under stirring, and kept stirred until the solution became very thick and uniform. The obtained suspension was left to stand at room temperature until the solution became a transparent gel.

In the abovementioned process, the gelatin enters a water pool of the enzyme body (reversed micelle) and gels there. In addition, since the gelatin having gelled in the water pool forms an intermolecular network, the whole mixture (nonaqueous phase) containing this enzyme body gels. Note that the enzymes (FₐₜₑDH and MDH) thermally denatured by heating can further be refolded by leaving the suspension to stand at room temperature.

In Example 5, the enzymes are solubilized in the water pool of the reversed micelle, and hence can evenly be dispersed within the reaction system (ionic liquid). In addition, since the enzymes are evenly dispersed within the ion gel formed from the enzymes/reversed micelle/ionic liquid, a high enzyme activity and high enzymatic catalytic efficiency can be obtained.

It was possible to obtain methanol from carbon dioxide at high fuel conversion efficiency by using the carbon dioxide fixation device of Example 5.

### Example 6

A carbon dioxide fixation device of Example 6 is a batch-type carbon dioxide fixation device and produces methanol as fuel from carbon dioxide, like the device of Example 4.

In Example 6, methanol is produced by a three-cascade enzymatic reaction using FₐₜₑDH, F_{ald}DH, and ADH as in Example 1.

In Example 6, glutamate dehydrogenase (GDH) as an enzyme for reducing an oxidant NAD⁺ of NADH, which is the mediator (coenzyme), to NADH was further dispersed in the nonaqueous phase. The enzyme body 11 containing these four enzymes was obtained by performing dispersion and immobilization by using a silica gel, and impregnating the obtained material with 3 mM NADH and a 0.1 M phosphoric acid buffer solution having a pH of 7, in the same manner as in Example 1. The obtained enzyme body 11 was dispersed in EMIMTFSI as a medium of the first nonaqueous phase.

The nonaqueous phase of the enzyme body/ionic liquid obtained by the above method was used as the nonaqueous phase 712 shown in FIG. 10, as in Example 4.

On the other hand, in Example 6, the nonaqueous phase 712 further contains glutamate dehydrogenase (GDH) as an enzyme for reducing NAD⁺ to NADH. Accordingly, it is possible to omit the cathode electrode 715 and anode electrode 714 shown in FIG. 10, and a reference electrode.

In Example 6, NADH, which is consumed when producing methanol by enzymatic reactions using the three enzymes, can rapidly be regenerated by the enzyme GDH. Therefore, the configuration of the carbon dioxide fixation device of Example 6 can prevent stopping of the enzymatic reactions caused by depletion of NADH, and can achieve a high methanol concentration.

Note that, glutamic acid, which is needed in the regeneration reaction of NADH by the enzyme GDH, can be supplied from the aqueous phase.

Furthermore, an ionic liquid was used as an absorbing medium of carbon dioxide in all of Examples 1 to 6 described above, so the solubility of carbon dioxide had greatly increased. This makes it possible to produce a high concentration of methanol as fuel by using the enzymatic reactions of Examples 1 to 6.

The carbon dioxide fixation device according to at least one embodiment and at least one example explained above includes a nonaqueous phase and aqueous phase. The nonaqueous phase contains an ionic liquid, enzyme body, and mediator. The enzyme body catalyzes a reduction reaction of carbon dioxide or its reduced product. The mediator acts as a reducing agent or coenzyme in this reduction reaction. Also, carbon dioxide or a reduced product thereof is supplied to the nonaqueous phase, and the abovementioned reduction reaction generates a reaction product. The aqueous phase contains an extraction liquid. The abovementioned reaction product is supplied from the nonaqueous phase to the aqueous phase. A configuration like this can provide a carbon dioxide fixation device and a fuel production system for efficiently fixing carbon dioxide.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A carbon dioxide fixation device comprising:
a nonaqueous phase which comprises an ionic liquid, an enzyme body that catalyzes a reduction reaction of one of carbon dioxide and a reduced product thereof, and a mediator that acts as one of a reducing agent and a coenzyme in the reduction reaction, the nonaqueous phase being configured to generate a reaction product by the reduction reaction; and
an aqueous phase which comprises an extraction liquid containing water, the aqueous phase being configured to have the reaction product supplied thereto from the nonaqueous phase.

2. The carbon dioxide fixation device according to claim 1, wherein the nonaqueous phase comprises a first nonaqueous phase configured to have carbon dioxide supplied thereto from a gaseous phase, the first nonaqueous phase being configured to generate a first reaction product by a reduction reaction of the carbon dioxide.

3. The carbon dioxide fixation device according to claim 1, wherein
the nonaqueous phase comprises:
a first nonaqueous phase configured to have carbon dioxide supplied thereto, the first nonaqueous phase being configured to generate a first reaction product by a reduction reaction of carbon dioxide;
a second nonaqueous phase configured to have the first reaction product supplied thereto, the second nonaqueous phase being configured to generate a second reaction product by a reduction reaction of the first reaction product; and
a third nonaqueous phase configured to have the second reaction product supplied thereto, the third nonaqueous phase being configured to generate a third reaction product by a reduction reaction of the second reaction product, and
the aqueous phase comprises:
a first aqueous phase configured to have the first reaction product supplied thereto from the first nonaqueous phase, the first aqueous phase being configured to supply the first reaction product to the second nonaqueous phase;
a second aqueous phase configured to have the second reaction product supplied thereto from the second nonaqueous phase, the second aqueous phase being configured to supply the second reaction product to the third nonaqueous phase; and
a third aqueous phase configured to have the third reaction product supplied thereto from the third nonaqueous phase.

4. The carbon dioxide fixation device according to claim 1, wherein
the nonaqueous phase comprises:
a first nonaqueous phase configured to have carbon dioxide supplied thereto, the first nonaqueous phase being configured to generate a first reaction product by a reduction reaction of carbon dioxide;
a second nonaqueous phase configured to have the first reaction product supplied thereto from the first nonaqueous phase, the second nonaqueous phase being configured to generate a second reaction product by a reduction reaction of the first reaction product; and
a third nonaqueous phase configured to have the second reaction product supplied thereto from the second nonaqueous phase, the third nonaqueous phase being configured to generate a third reaction product by a reduction reaction of the second reaction product, and
the carbon dioxide fixation device is configured to supply the third reaction product from the third nonaqueous phase to the aqueous phase.

5. The carbon dioxide fixation device according to claim 3, wherein the carbon dioxide fixation device is configured to supply carbon dioxide from a gaseous phase to the first nonaqueous phase.

6. The carbon dioxide fixation device according to claim 2, wherein the carbon dioxide fixation device is configured to supply carbon dioxide from atmosphere to the first nonaqueous phase.

7. The carbon dioxide fixation device according to claim 1, further comprising a separator disposed between the nonaqueous phase and the aqueous phase, the separator comprising a permeation-separation membrane.

8. The carbon dioxide fixation device according to claim 1, wherein the aqueous phase further comprises a proton source which supplies protons to the nonaqueous phase.

9. The carbon dioxide fixation device according to claim 1, wherein at least one of the enzyme bodies is selected from the group consisting of an enzyme, a first composite comprising an enzyme and a molecular aggregate that comprises a dispersant, a microcapsule comprising an enzyme-including core and a shell covering the core, a cell comprising an enzyme, a microorganism comprising an enzyme, and a second composite comprising a support and an enzyme immobilized by the support.

10. The carbon dioxide fixation device according to claim 1, further comprising an external flow path through which the aqueous phase flows.

11. The carbon dioxide fixation device according to claim 1, further comprising a plurality of single-cells each comprising the nonaqueous phase and the aqueous phase,
the plurality of single-cells being stacked to form a stacked structure.

12. The carbon dioxide fixation device according to claim 1, wherein the nonaqueous phase comprises a porous membrane having the enzyme body dispersed therein or supported thereon, the porous membrane being wound.

13. The carbon dioxide fixation device according to claim 12, wherein
the wound porous membrane is wound a plurality of times such that a plurality of layer sections are arranged along a radial direction,
the enzyme body comprises a plurality of enzyme bodies, and
the plurality of enzyme bodies are dispersed or supported among the plurality of layer sections such that different enzyme bodies are arranged along the radial direction.

14. The carbon dioxide fixation device according to claim 1, further comprising one or more pairs of electrodes disposed at the nonaqueous phase.

15. The carbon dioxide fixation device according to claim 14, further comprising a reference electrode disposed at the aqueous phase.

16. A fuel production system comprising:
a fuel generation section comprising the carbon dioxide fixation device according to claim 1;
a carbon dioxide supply section configured to supply carbon dioxide to the fuel generation section; and
an extraction liquid recovery section configured to recover the reaction product from the fuel generation section.

17. The fuel production system according to claim 16, further comprising:
one or more pairs of electrodes disposed at the nonaqueous phase; and
a cell voltage control section configured to control voltage applied to the one or more pairs of electrodes.

18. The fuel production system according to claim 16, further comprising:
one or more pairs of electrodes disposed at the nonaqueous phase; and
an electric current control section configured to control an electric current which flows between the one or more pairs of electrodes.

19. The fuel production system according to claim 16, further comprising a temperature controller configured to control a temperature in the fuel generation section.

20. The fuel production system according to claim 16, wherein the carbon dioxide supply section is configured to control a pressure of a gas containing carbon dioxide supplied to the fuel generation section.
